(19) United States
(12) Patent Application Publication
(10) Pub. No.: US 2014/0004201 A1
(43) Pub. Date: Jan. 2, 2014

(54) Treatment of Vascular Diseases Using Encapsulated Cells Encoding and Secreting GLP-1, or a Fragment or Variant Thereof

(71) Applicants: Andrew Lewis, Surrey (GB); Peter Stratford, Surrey (GB); Peter Geigle, Alzenau (DE); Christine Wallrapp, Grossostheim (DE)

(72) Inventors: Andrew Lewis, Surrey (GB); Peter Stratford, Surrey (GB); Peter Geigle, Alzenau (DE); Christine Wallrapp, Grossostheim (DE)

(73) Assignee: Biocompatibles UK Ltd., Farnham, Surrey (GB)

(21) Appl. No.: 13/822,806

(22) PCT Filed: Sep. 15, 2011
(86) PCT No.: PCT/EP11/04646

§ 371 Date: Aug. 20, 2013

(30) Foreign Application Priority Data
Sep. 15, 2010 (EP) 10009629.6

(51) Int. Cl. A61K 9/50; A61K 35/28

(52) U.S. Cl. CPC A61K 9/5089; A61K 35/28
USPC 424/490; 424/93.7

(57) ABSTRACT

The present application refers to the use of cells, e.g. mesenchymal stem cells or mesenchymal stromal cells, or any further suitable cell, encoding and secreting at least GLP-1, or a fragment or variant thereof, and preferably additionally secreting VEGF, for the prevention, treatment and/or amelioration of vascular diseases, wherein the cells, encoding and secreting at least GLP-1, or a fragment or variant thereof, and preferably additionally secreting VEGF, are encapsulated in a (spherical) microcapsule to prevent a response of the immune system of the patient to be treated. The present application also refers to the use of these (spherical) microcapsule(s) or of a pharmaceutical composition containing these cells or (spherical) microcapsule(s) for the prevention, treatment and/or amelioration of vascular diseases.

The present application refers to the use of cells, e.g. mesenchymal stem cells or mesenchymal stromal cells, or any further suitable cell, encoding and secreting at least GLP-1, or a fragment or variant thereof, and preferably additionally secreting VEGF, for the prevention, treatment and/or amelioration of vascular diseases, wherein the cells, encoding and secreting at least GLP-1, or a fragment or variant thereof, and preferably additionally secreting VEGF, are encapsulated in a (spherical) microcapsule to prevent a response of the immune system of the patient to be treated. The present application also refers to the use of these (spherical) microcapsule(s) or of a pharmaceutical composition containing these cells or (spherical) microcapsule(s) for the prevention, treatment and/or amelioration of vascular diseases.

Many patients worldwide suffer from vascular diseases, a sort of disease which typically affects or leads to a pathological state of large and medium sized muscular arteries and the tissues they supply. It is usually triggered by endothelial cell dysfunction and may include conditions that affect the circulatory system, veins and lymph vessels but also blood disorders that affect circulation. In some cases factors like pathogens may trigger such vascular diseases, whereby for example, oxidized LDL particles and other inflammatory stimuli activate endothelial cells and change their secretion pattern. Endothelial cells may then start to secrete cytokines and chemokines and express adhesion molecules on their surface. This in turn may result in recruitment of white blood cells (monocytes and lymphocytes), which can infiltrate the blood vessel wall with subsequent stimulation of the smooth muscle cell layer with cytokines produced by endothelial cells and recruited white blood cells. Additionally, smooth muscle cells may proliferate and migrate towards the blood vessel lumen. Such a process may cause thickening of the vessel wall, forming plaques consisting of proliferating smooth muscle cells, macrophages and various types of lymphocytes. Occurrence of plaques typically results in obstructed blood flow leading to diminished amounts of oxygen and nutrients that reach the target organ. In the final stages, plaques may also cause a rupture of the cascular diseases causing the formation of clots.

Some prominent conditions that fall under the category of “vascular diseases” are e.g. peripheral vascular disease, aneurysm, renal artery disease, Raynaud's phenomenon (also called Raynaud's disease or Raynaud's syndrome), Buerger's disease, peripheral venous disease, varicose veins, venous blood clots, deep vein thrombosis (DVT), pulmonary embolism, chronic venous insufficiency, and other vascular conditions such as e.g., blood clotting disorders, lymphedema, vein graft disease, etc.

In this context one prominent vascular disease comprises vein graft diseases. The term “vein graft disease” is a generic reference to the progressive degradation and build up of atheroma and clots within the ever-thickening wall of veins which are used as arteries during surgical bypass operations. Often, over days to less than a decade, the sections of veins which are used as bypass graphs (sewn into the side of arteries as another path for blood to flow through) deform, narrow and occlude.

Vascular diseases may also comprise aneurysms. An aneurysm is usually an abnormal bulge in the wall of a blood vessel. Such aneurysms can form in any blood vessel, but occur most commonly in the aorta (aortic aneurysm) which is the main blood vessel leaving the heart, e.g. the thoracic aortic aneurysm (part of aorta in the chest), the abdominal aortic aneurysm, including suprarenal aneurysm (involving the arteries above the kidneys), juxtarenal aneurysm (involving the main renal arteries), and infrarenal aneurysm (involving the arteries below the kidneys). There is an increased risk of atherosclerotic plaque (fat and calcium deposits) formation at the site of the aneurysm, clot (thrombus) formation and shedding at the site of the aneurysm, but the most severe cases increase in size and may lead to rupture.

Renal artery disease is most commonly caused by atherosclerosis of the renal arteries (see above). It occurs in people with generalized vascular disease. Less often, renal artery disease can be caused by fibromuscular dysplasia, a congenital (present at birth) abnormal development of the tissue that makes up the renal arteries. This type of renal artery disease occurs in younger age groups.

Two further prominent vascular diseases known in the above context are Raynaud's phenomenon (also called Raynaud's disease or Raynaud's syndrome) and Buerger's disease. Raynaud's phenomenon consists of spasms of the small arteries of the fingers, and sometimes, the toes, brought on by exposure to cold or excitement. Certain occupational exposures bring on Raynaud's phenomenon. The episodes produce temporary lack of blood supply to the area, causing the skin to appear white or bluish and cold or numb. In some cases, the symptoms of Raynaud's phenomenon may be related to underlying connective tissue disorders (i.e., lupus, rheumatoid arthritis, scleroderma). Buerger's disease most commonly affects the small and medium sized arteries, veins, and nerves. Although the trigger and the mechanism are unknown, there is a strong association with tobacco use or exposure. The arteries of the arms and legs become narrowed or blocked, causing lack of blood supply (ischemia) to the fingers, hands, toes and feet. Pain occurs in the arms, hands, and more frequently the legs and feet, even at rest. With severe blockages, the tissue may die (gangrene), requiring amputation of the fingers and toes. Superficial vein inflammation and symptoms of Raynaud's phenomenon occur commonly in patients with Buerger's Disease.

Further important vascular diseases known in the above context comprise peripheral diseases, such as peripheral vascular disease (PVD), commonly referred to as peripheral arterial disease (PAD) or peripheral artery occlusive disease (PAOD), which refers to the obstruction of large arteries not within the coronary, aortic arch vasculature, or brain. PVD can result from atherosclerosis, inflammatory processes leading to stenosis, an embolism, or thrombus formation, the build-up of fat and cholesterol deposits, called plaque, on the inside walls of peripheral arteries (blood vessels outside the heart). Over time, the build-up narrows the artery and may eventually lead to an obstructed blood flow. Diminished amounts of oxygen and nutrients reaching the target organ due to lack of blood flow in the body's tissue typically lead to ischemia (acute or chronic ischemia). A blockage in the carotid arteries (the arteries supplying the brain) can additionally lead to a transient ischemic attack (TIA) or stroke. A blockage in the legs can lead to leg pain or cramps with activity (claudication), changes in skin color, sores or ulcers and feeling tired in the legs. Total loss of circulation can lead to gangrene and loss of a limb. Finally, a blockage in the renal arteries can cause renal artery disease (stenosis). The symptoms include uncontrolled hypertension (high blood pressure), congestive heart failure, and abnormal kidney function. PAD is a term used to refer to atherosclerotic blockages found in the lower extremity.

Another peripheral disease in the context of vascular diseases is the so called peripheral venous disease. Veins are flexible, hollow tubes with flaps inside, called valves. When muscles contract, the valves open, and blood moves through the veins. When muscles relax, the valves close, keeping blood flowing in one direction through the veins. However, if the valves inside the veins become damaged, the valves may not close completely. This allows blood to flow in both directions. When muscles relax, the valves inside the damaged vein(s) will not be able to hold the blood, causing the pooling of blood or swelling in the veins, that is a typical effect of peripheral venous disease. The blood begins to move more slowly through the veins and it may stick to the sides of the vessel walls and blood clots can form. Peripheral venous disease may also lead to so called varicose veins. Varicose veins are bulging, swollen, purple, ropy veins, seen just under the skin, caused by damaged valves within the veins. There are many further vascular diseases, which may be identified in this context. Many of the treatments doe vascular diseases focus on treating the consequences of the disease and not the cause, for instance, prescribing aspirin or thrombolytics to stop blood clotting in the diseased vessels. This invention addresses the underlying biology of the disease, treating the cause and not the consequences.

One specific invasive treatment comprises e.g. bypass grafting. The success of such a bypass grafting, in particular coronary artery bypass grafting, is usually limited by its poor long-term graft patency. Despite the superior patency of arterial grafts, saphenous vein remains the most commonly used conduit for coronary artery bypass because of its predictable handling qualities and ready availability (see The Society of Cardiothoracic Surgeons of Great Britain and Ireland National Adult Cardiac Surgical Database Report 2003. Dendrite Clinical Systems, Oxforshire, United Kingdom). Over 40% of vein grafts are thrombosed at 10 years postoperatively however (see Goldman S, Zadina K, Moritz T, Ovitt T, Sethi G, Copeland J G, Thottapurathu L, Krasnicka B, Ellis N, Anderson R J, Henderson W. VA Cooperative Study Group. Long-term patency of saphenous vein and left internal mammary artery grafts after coronary artery bypass surgery results from a Department of Veterans Affairs Cooperative Study. J Am Coll Cardiol. 2004; 44; 2149-56; and Sabik J F, Lytle B W, Blackstone E H, Houghtaling P L, Cosgrove D M. Comparison of saphenous vein and internal thoracic artery graft patency by coronary system. Ann Thorac Surg. 2005; 79; 544-51) largely as a consequence of vein graft disease that is characterised by neointima formation, atherosclerosis, plaque rupture and graft thrombosis. Graft failure results in major adverse cardiac events and leads to repeat revascularisation procedures. With the exception of aggressive lipid lowering (see Campeau L, Hunninghake D B, Knatterud G L, White C W, Domanski M, Forman S A, Forrester J S, Geller N L, Gobel F L, Herd J A, Hoogwerf B J, Rosenberg Y. Aggressive cholesterol lowering delays saphenous vein graft atherosclerosis in women, the elderly, and patients with associated risk factors. NHLBI post coronary artery bypass graft clinical trial. Post CABG Trial Investigators. Circulation. 1999; 99:3241-7), no therapy has been shown to improve long-term vein graft patency in clinical studies.

Vein grafts subjected to arterial pressure and flow demonstrate proliferation of vascular smooth muscle cells within the media and adventitia. These migrate towards the lumen leading to the formation of a neointimal layer between the endothelium and vessel media that provides a soil for macrophage foam cell accumulation and the development of atherosclerotic plaques. It has been shown that inhibition of early neointima formation in experimental vein grafts inhibits subsequent foam cell accumulation and atherogenesis (see Angelini G D, Lloyd C, Bush R, Johnson J, Newby A C. An external, oversized, porous polyester stent reduces vein graft neointima formation, cholesterol concentration, and vascular cell adhesion molecule 1 expression in cholesterol-fed pigs. J Thorac Cardiovasc Surg. 2002; 124:950-956; and Ehsan A, Mann J, Dell'Acqu G, Dzau V J. Long-term stabilization of vein graft wall architecture and prolonged resistance to experimental atherosclerosis after E2F decoy oligonucleotide gene therapy. J Thorac Cardiovasc Surg. 2001; 121:714-22). This has formed the basis of recent therapeutic strategies in vein graft disease (see PREVENT IV Investigators. Efficacy and safety of edifoligide, an E2F transcription factor decoy, for prevention of vein graft failure following coronary artery bypass graft surgery: PREVENT IV: A randomized controlled trial. JAMA. 2005; 294:2446-54; and Murphy G J, Newby A C, Jeremy J Y, Baumbach A, Angelini G D. A randomised trial of an external dacron sheath for the prevention of vein graft disease: The Extent Study. J Thorac Cardiovasc Surg. 2007; 134:504-5). Unfortunately, stripping of the vein graft from the leg also disrupts the microvasculature within the vein wall and the resulting hypoxia accelerates the atherosclerotic process. It has been previously demonstrated that vein graft disease can be inhibited in the swine model in the long-term by the application of periadventitial macroporous Dacron sheaths (see George S J, Izzat M B, Gadsdon P, Johnson J L, Yim A P, Wan S, Newby A C, Angelini G D, Jeremy J Y. Macro-porosity is necessary for the reduction of neointimal and medial thickening by external stenting of porcine saphenous vein bypass grafts. Atherosclerosis. 2001; 155:329-6). These promote the formation of a highly vascularised neoadventitia that prevents graft hypoxia and reduces neotimal proliferation. Nevertheless, this technique failed to translate into clinical benefits in a recent study however due to graft thrombosis attibuted to graft kinking within the semi-rigid external sheaths (see Murphy G J, 2008, supra). The application of periadventitial microbeads eluting the antiproliferative agent rapamycin have also been studied. In this case early inhibition of vascular smooth muscle proliferation at 1 week and neointima formation at four weeks was accompanied by inhibition of adventitial neoangiogenesis. A subsequent acceleration of VSMC proliferation at 4 weeks led to a catch up phenomenon and a worsening of graft atherosclerosis in the long-term (see Rajathurai T, Rizvi S I, Lin H, Angelini G D, Newby A C, Murphy G J. Peri-adventitial rapamycin eluting microbeads promote vein graft disease in long-term pig vein-into-artery interposition grafts. Circulation: Cardiovascular Interventions 2010; 3:157-65).

There are only very few advanced treatments for vascular diseases in development. Trinam® is a novel product from Ark Therapeutics consisting of a local delivery device and a gene-based medicine, being developed to prevent the blocking of blood vessels that frequently occurs after vascular surgery. Trinam® is a combination of a vascular endothelial growth factor (VEGF-D) gene packaged in an adenoviral vector (Ad 5) and a bio-degradable local drug delivery device made from collagen. At the end of access graft surgery, the delivery device is fitted around the outside of the patient's vein where it has been joined to the access graft. The adenoviral vector carrying the VEGF gene is then injected into a space between the device and the blood vessel. The administration of the gene to the outside of the blood vessel rather than into the blood supply localises delivery of the gene to the target tissue site (smooth muscle cells) and reduces the risk of unwanted systemic effects. Once the VEGF gene is transfected locally, muscle cells in the vessel wall produce the VEGF protein which triggers the release of beneficial nitric oxide and prostacyclin, keeping blood vessel walls in a healthy state and regulating muscle cell growth to prevent blocking of the vessel.

In summary, at present there appear to be only few efficient therapies available in the art for the treatment of vascular diseases as described above, which allow efficiently preventing, treating or ameliorating such a disease in a patient to be treated without adverse side effects and avoiding repeated administration.

Therefore, it is an objective of the present invention to provide a further or alternative efficient therapy for treatment of vascular diseases, which provides a long-term effect in vivo without the need of repeated administration and/or the risk of evoking an undesired immune response.

The object underlying the present invention is solved by the attached claims, particularly by the use of cells, e.g. mesenchymal stem cells or mesenchymal stromal cells, or any further cell, that may be used in the context of the present invention, encoding and secreting at least GLP-1, or a fragment or variant thereof, and preferably additionally secreting VEGF, for the treatment of vascular diseases or diseases related thereto, wherein the cells, encoding and secreting at least the factors GLP-1 and preferably VEGF, or a fragment or variant thereof, are encapsulated in a (spherical) microcapsule to prevent a response of the immune system of the patient to be treated. In the context of the present invention the term “cells encoding . . . ” typically means “cells, which are engineered to contain or comprise nucleic acids encoding . . . ”.

The present inventors surprisingly found that it is possible to treat efficiently vascular diseases by utilizing encapsulated cells secreting angiogenic factors, particularly GLP-1, in the treatment of vascular diseases, more preferably to utilize its angiogenic effects and its capabilities to powerfully reduce damage caused by ischemia or oxygen shortage, or the neovascular properties of e.g. VEGF, etc., without the need of repeated administration of such factors and/or the risk of an undesired immune response against e.g. implanted allogenic cells expressing those factors.

In this context, the effects of GLP-1 on angiogenesis have not been extensively studied before and it has not been expected to be one of its primary mechanisms of action given its very short half-life in the circulation. The presence of the GLP-1 receptor in human coronary artery endothelial cells (HCAECs) and the ameliorative actions of GLP-1 on endothelial dysfunction in type 2 diabetic patients has however been shown (see Erdogdu O, Nathanson D, Sjoholm A, Nystrom T, Zhang Q., Exendin-4 stimulates proliferation of human coronary artery endothelial cells through eNOS-, PKA- and PI3K/Akt-dependent pathways and requires GLP-1 receptor. Mol Cell Endocrinol. 2010 Aug. 30; 325(1-2):26-35.) Erdogu et al. have studied the effect of exendin-4 on cell proliferation and its underlying mechanisms in HCAECs. Incubation of HCAECs with exendin-4 resulted in a dose-dependent increase in DNA synthesis and an increased cell number, associated with an enhanced eNOS and Akt activation, which were inhibited by PKA, PI3K, Akt or eNOS inhibitors and abolished by a GLP-1 receptor antagonist. Similar effects were obtained by applying GLP-1 (7-36) or GLP-1 (9-36). Co-incubation of exendin-4 and GLP-1 did not show additive effects. Their results suggest that both exendin-4 and GLP-1 stimulate proliferation of HCAECs through PKA-PI3K/Akt-eNOS activation pathways via a GLP-1 receptor-dependent mechanism. According to the inventor's hypothesis, without being bound thereto, there is therefore a very good rationale for why the co-expression of GLP-1 and other pro-angiogenic factors such as VEGF can work synergistically to induce angiogensis.

The cells used for providing the herein described inventive solution, encoding and secreting at least GLP-1, or a fragment or variant thereof, and preferably additionally secreting VEGF, for the treatment of a vascular disease or diseases related thereto, are preferably encapsulated in a (spherical) microcapsule to prevent a response of the immune system of the patient to be treated. In the context of the present invention, such a (spherical) microcapsule preferably comprises a (spherical) core (i.e. the core may be spherical or not) and at least one surface coating layer, wherein:
the (spherical) core comprises or consists of (a mixture of) cross-linked polymers and cells, e.g. mesenchymal stem cells or mesenchymal stromal cells, or any further cell (type), that may be used in the invention, encoding and secreting at least GLP-1, or a fragment or variant thereof, as defined herein, and preferably additionally secreting VEGF; and
the at least one surface coating layer comprises or consists of (a mixture of) of typically cross-linked polymers.

The (spherical) microcapsule, comprising cells as used herein encoding and secreting at least GLP-1, or a fragment or variant thereof, as defined herein, and preferably additionally secreting VEGF, typically comprises a particle size, herein referred to as the total diameter of the (spherical) microcapsule. Generally, the total diameter of the (spherical) microcapsule as used herein may vary considerably depending on the specific treatment and administration mode. In the context of the present invention, the treatment typically occurs locally by administration of the (spherical) microcapsule as used herein into a specific administration site, e.g. by injection or implantation but also may occur systemically by administering the (spherical) microcapsule as used herein systemically, e.g. via parenteral injection. Accordingly, the administration mode may limit the total diameter of the (spherical) microcapsule as used herein, e.g. by the diameter of the injection cannula. The total diameter of the (spherical) microcapsule as used herein is furthermore determined by the diameter of the core of the (spherical) microcapsule as well as by the thickness of the at least one surface coating layer(s), as both diameters typically depend at least in part on each other and of course, influence the total diameter of the (spherical) microcapsule.

For the treatment of a vascular disease as defined herein and diseases related thereto, the inventors of the present application have surprisingly found, that a total diameter (particle size) of the (spherical) microcapsule of about 100 μm to about 800 μm, preferably of about 100 μm to about 700 μm, more preferably a total diameter of about 100 μm to about 500 μm, and even more preferably a total diameter of about 100 μm to about 400 μm or a total diameter of about 100 μm to about 300 μm or even a total diameter of about 100 μm to about 200 μm may be used. Particularly, a total diameter (particle size) of the (spherical) microcapsule of about 100 μm to about 300 μm, more preferably a total diameter of about 110 μm to about 250 μm, even more preferably a total diameter of about 120 μm to about 225 μm, and most preferably a total diameter of about 130 μm to about 200 μm, e.g. about 110, 120, 130, 140, 150, 160, 170, 180, 190 or 200 μm, is advantageous for the treatment of a vascular disease as defined herein. (Spherical) microcapsules, comprising such a total diameter, are typically retained in the site of administration and do not migrate into the surrounding tissue. This allows providing a continuous expression of secreting at least GLP-1, or a fragment or variant thereof, as defined herein, and preferably additionally secreting VEGF, at the site of injection during treatment for a sufficient period of time to provide the entire spectrum of beneficial effects known for these factors, e.g. their herein described bioactivity.

In the herein context, the term “spherical” is understood in its broadest meaning. A spherical particle is preferably understood to have a sphere-like shape, whereby the shape may be symmetrical or asymmetrical, e.g. a (spherical) microcapsule and/or its core may have ellipsoidal shape. In a less preferred embodiment the microcapsule or core used according to the present invention may not be spherical within the herein meaning, but may have an arbitrary shape with e.g. protruding or invading segments on the surface of the microcapsule. Where ever in the present disclosure “spherical” microcapsules or cores are mentioned, “non-spherical” microcapsules or cores may be provided, prepared or used as well.

The (spherical) microcapsule as defined herein preferably comprises a (spherical) core (i.e. the core may be spherical or not), wherein the (spherical) core comprises or consists of (a mixture of) cross-linked polymers and cells, e.g. mesenchymal stem cells or mesenchymal stromal cells, or any other cell (type), that may be used in the context of the present invention, encoding and secreting at least GLP-1, or a fragment or variant thereof, as defined herein, and preferably additionally secreting VEGF, for treatment of a vascular disease, as defined herein, or diseases related thereto.

In the context of the present invention, the typically cross-linked polymers of the (spherical) core of the (spherical) microcapsule form a scaffold structure embedding the cells, e.g. mesenchymal stem cells or mesenchymal stromal cells, or any other cell (type), that may be used in the context of the present invention, in its cavities. These cells may be embedded in the scaffold structure individually or, typically, as aggregates, e.g. as (a pool of) aggregated cells of about 10 to about 10,000 cells, e.g. about 10 to about 100, about 10 to about 200, about 10 to about 300, about 10 to about 400, about 10 to about 500, about 10 to about 1,000, about 10 to about 5000 or about 10 to about 10,000 cells, more preferably 10 to about 100 or 10 to about 200 cells, even more preferably of about 30 to about 80 or about 60 to about 70 cells per (spherical) core. Preferably, the (spherical) core comprises a homogenous distribution of the cross-linked polymers and of embedded cells as defined herein. Preferably, the core, including the scaffold structure and the embedded cells as defined herein, is prepared according to a method as disclosed below. In this context, it is of critical importance to embed the encapsulated cells of the (spherical) microcapsule, e.g. mesenchymal stem cells or mesenchymal stromal cells, autologous cells or any other cell (type), which may be used in the context of the present invention, entirely in the polymer matrix when preparing (spherical) microcapsules for the use according to the present invention.

The embedded cells, e.g. mesenchymal stem cells or mesenchymal stromal cells, or any other cell (type) as defined herein, that may be used for the (spherical) microcapsule in the context of the present invention, may be present in a solution containing the (spherical) microcapsule, preferably in the core of the (spherical) microcapsule, in a concentration of about 1×10⁵ to about 5×10⁸ cells/100 μl, about 1×10⁶ to about 5×10⁸ cells/100 μl or about 1×10⁷ cells/μl to about 5×10⁸ cells/100 μl, more preferably in a concentration of about 1×10⁵ to about 5×10⁶ cells/100 μl, about 1×10⁶ to about 5×10⁷ cells/100 μl, or about 1×10⁷ cells/μl to about 5×10⁸ cells/100 μl, and most preferably in a concentration of about 1×10⁵ to about 5×10⁶ cells/100 μl.

The cells embedded in the (spherical) core of the (spherical) microcapsule is typically dependent on the diameter of the (spherical) core as defined above. As an example, an exemplary inventive (spherical) microcapsules having a total diameter of about 160 μm may comprise in its (spherical) core a number of embedded cells, e.g. mesenchymal stem cells or mesenchymal stromal cells, or any other cell (type) as defined herein, e.g. of about e.g. 10 to 100, preferably of about 30 to 80, e.g. about 60 to 70 cells per (spherical) core and thus per (spherical) microcapsule. Accordingly, administration of about 60,000 inventive (spherical) microcapsules typically provides about 3 to 4 million cells at once into the site to be treated.

The core of the (spherical) microcapsule used according to the present invention typically has a diameter (particle size) of not more than the diameter of the total diameter of the (spherical) microcapsule as defined herein. Typically, the core of the (spherical) microcapsule used according to the present invention has a diameter as defined above for the total diameter of the inventive (spherical) microcapsule, more typically a diameter of about 50 μm to about 220 μm, preferably a diameter of about 60 μm to about 200 μm, likewise preferably a diameter of about 70 μm to about 180 μm, more preferably a diameter of about 80 μm to about 160 μm, and even more preferably a diameter of about 80 μm to about 155 μm, e.g. about 80, 85, 90, 95, 100, 105, 110, 115, 120, 125, 130, 135, 140, 145, 150 or 155 μm or a range defined by any combination of two of these values. Particularly preferred, the core of the (spherical) microcapsule, as used according to the present invention, has a diameter, which is preferably about 10 to about 120 μm less than the total diameter of the (spherical) microcapsule as defined herein, more preferably about 15 to about 110 μm less than the total diameter of the (spherical) microcapsule as defined herein, and most preferably about 20 to about 100 μm less than the total diameter of the (spherical) microcapsule as defined herein, e.g. about 20 to about 90 μm, about 20 to about 80 μm, about 20 to about 70 μm, or about 30 to about 70 μm. In other words, the diameter of the core of the (spherical) microcapsule, as used according to the present invention, may have a size of about 10 μm, of about 20 μm, of about 30 μm, of about 40 μm, of about 50 μm, of about 60 μm, of about 70 μm, of about 80 μm, of about 90 μm, of about 100 μm, of about 110 μm, of about 120 μm, of about 125 μm, of about 130 μm, of about 135 μm, of about 140 μm, of about 145 μm, of about 150 μm, of about 155 μm, of about 160 μm, of about 165 μm, of about 170 μm, of about 175 μm, of about 180 μm, of about 185 μm, of about 190 μm, of about 195 μm, of about 200 μm, of about 205 μm, of about 210 μm, of about 215 μm, or even of about 220 μm, or may comprise any range selected from any two of the herein mentioned specific values.

The core of the (spherical) microcapsule as defined herein comprises cells, encoding and secreting at least GLP-1, or a fragment or variant thereof, as defined herein, and preferably additionally secreting VEGF, for treatment of a vascular disease, as defined herein, or diseases related thereto. Such cells, e.g. mesenchymal stem cells or mesenchymal stromal cells, or any other cell (type), that may be used in the context of the present invention for the (spherical) core, being located at the core periphery or cells protruding out of the scaffold structure may evoke immunological problems, since the immune system will recognize these microcapsules as foreign components and, thus, these microcapsules will be attacked by the immune system.

Although this effect may be avoided by lowering the cell concentration in the initial solution, the present invention allows improving the efficacy of the microcapsule by increasing the core's cell portion. The higher the concentration of cells in the core, the smaller the total volume of the resultant microcapsules to be transplanted, i.e. the more efficient the microcapsules may work at the site of injection. In order to avoid immunological problems when using high concentrations of cells in the (spherical) core of the (spherical) microcapsule, the invention provides at least one surface coating layer applied on the (spherical) core. This surface coating layer does not allow an immune response to occur, even if cells are located very closely to the core periphery, since these cells are not accessible for the host's immune system due to the surface coating layer acting as a barrier. This surface coating layer is typically composed (of a mixture) of a usually cross-linked polymer as defined herein, which does not contain any cells. According to a particular preferred embodiment the afore defined (spherical) core is coated with at least one or more than one surface coating layer(s), e.g. with 1, 2, 3, 4, 5, 5-10 or more surface coating layer(s), more preferably 1, 2 or 3 surface coating layer(s), most preferably with only one surface coating layer or with only two surface coating layers. Typically, each surface coating layer comprises a uniform thickness around the core. The thickness of the surface coating layer(s) of the (spherical) microcapsule, as used according to the present invention, may be varied almost arbitrarily and is typically in a range of about 10 to about 120 μm, preferably in a range of about 15 to about 110 μm, and even more preferably in a range of about 20 to about 100 μm less than the total diameter of the (spherical) microcapsule as defined herein, e.g. in a range of about 20 to about 90 μm, of about 20 to about 80 μm, of about 20 to about 70 μm, or of about 30 to about 70 μm. In other words, the thickness of the surface coating layer(s) of the (spherical) microcapsule, as used according to the present invention, may have a size of about 10 of about 20 μm, of about 30 μm, of about 40 μm, of about 50 μm, of about 60 μm, of about 70 μm, of about 80 μm, of about 90 μm, of about 100 μm, of about 110 μm, of about 120 μm, of about 125 μm, of about 130 μm, of about 135 μm, of about 140 μm, of about 145 μm, of about 150 μm, of about 155 μm, of about 160 μm, of about 165 μm, of about 170 μm, of about 175 μm, of about 180 μm, of about 185 μm, of about 190 μm, of about 195 μm, of about 200 μm, of about 205 μm, of about 210 μm, of about 215 μm, or even of about 220 μm, or may comprise any range selected from any two of the herein mentioned specific values.

The (spherical) core of the (spherical) microcapsule as used herein (and optionally of the at least one surface coating of the (spherical) microcapsule) comprises or consists of (a mixture of) cross-linked polymers. In this context, any pharmaceutically acceptable (cross-linkable) polymer known in the art and being suitable for encapsulation may be used for the formation of the (spherical) core and, independent from each other, the at least one surface coating layer(s) of the (spherical) microcapsule, as defined according to the present invention. Preferably, such polymers are used, which, on the one hand, are permeable in their cross-linked state for supply of oxygen and nutrients from outside, and, on the other hand, allow diffusion of the peptide(s) encoded and secreted by the core cells from the microcapsule into the patient's tissue or body fluids. Furthermore, the cross-linked polymers prevent intrusion of components of the body's immune system through the matrix. By way of example, polymers may be used such as synthetic, semi-synthetic and natural water-soluble (bio)polymers, e.g. from natural polymers such as selected proteins or polymers based on proteins (e.g. collagens, albumins etc.), polyamino acids (e.g. poly-L-lysine, poly-L-glutamic acid, etc.), polysaccharides and their derivatives (e.g. carboxylmethyl cellulose, cellulose sulfate, agarose, alginates including alginates of brown algae (e.g. of species Laminarales, Ectocarpales, Fucales), carrageenans, hyaluronic acid, heparin and related glycosamino sulfates, dextranes and its derivatives, chitosan and their derivatives). Synthetic polymers may also be used such as e.g. aliphatic polyesters (e.g. polylactic acid, polyglycolic acid, polyhydroxybutyrates, etc.), polyamides, polyanhydrides, polyorthoesters, polyphosphazenes, thermoplastic polyurethanes, polyvinyl alcohols, polyhydroxyethylmethacrylates, polymethylmethacrylates and polytetrafluoroethylenes, etc.

Furthermore, block polymers may be used herein accordingly, i.e. polymers derived by combination of two or more of the aforementioned polymers. Such block polymers may be selected by a skilled person depending on the desired properties, e.g. pore size, cross-linking status, toxicity, handling, biocompatibility, etc. Any of the herein polymers is defined as a “chemically different polymer” in the context of the present invention, i.e. each of these polymers typically does not exhibit an identical molar mass and structure with any other of the herein polymers. In contrast, “chemically identical polymers” means, that the polymers exhibit an identical molar mass and structure.

Finally, mixtures of the herein polymers are also encompassed herein, wherein the amounts of polymers contained in such a mixture may be selected by a skilled person depending on the desired properties, e.g. as outlined herein. In this respect, mixtures of polymers may be regarded as chemically identical to another polymer mixture (“chemically identical polymers”), if the overall molar mass of the resultant polymer mixture and the corresponding molar percentage of the single polymers of the mixture are identical to the other polymer mixture.

Preferably, the (mixture of) cross-linked polymers of the (spherical) core of the (spherical) microcapsule as used herein (and optionally of the at least one surface coating layer of the (spherical) microcapsule) comprise or consist of alginate(s). Alginates, if used according to present invention as a polymer for the formation of the (spherical) core and/or of the at least one surface coating layer are particularly advantageous due to their biocompatibility and cross-linking properties. From a chemical point of view, alginates are anionic polysaccharides derived from homopolymeric groups of β-D-mannuronic acid and α-L-guluronic acid, separated by heteropolymeric regions of both acids. Alginates are water soluble and form high viscosity solutions in the presence of monovalent cations such as sodium or potassium. A cross-linked water insoluble hydrogel is formed upon interaction of single alginate chains with bi-, tri- or multivalent cations (such as calcium, barium or polylysine). Preferably, purified alginates (e.g. according to DE 198 36 960, the specific disclosure of which is incorporated herein by reference) are used for encapsulation, more preferably potassium or sodium alginates in physiological saline solution. Such alginates typically exhibit an average molar mass of about 20 kDa to about 10,000 kDa, more preferably a molar mass of about 100 kDa to about 1,200 kDa. Alginates used for the formation of the core and/or of the at least one surface coating layer of the (spherical) microcapsule as used according to the present invention, may be provided as a solution, more preferably as an aqueous solution. The viscosity of a 0.2% (w/v) aqueous alginate solution of the alginate to be used may be in the range of about 2 to about 50 mPa s, more preferably in the range of about 3 to about 10 mPa s. If alginates are used according to the present invention, those, which are rich in α-L-guluronic acid, are preferred. In other words, alginates containing at least 50% α-L-guluronic acid (and less than 50% β-D-mannuronic acid) are preferred. More preferably, the alginate to be used contains 50% to 70% α-L-guluronic acid and 30 to 50% β-D-mannuronic acid. Alginates suitable for preparing (spherical) microcapsules as used according to the present invention are obtainable by extraction from certain algae species including, without being limited thereto, brown algae, e.g. Laminarales, Ectocarpales, Fucales, etc., and other species of algae producing alginates. Alginates may be isolated from fresh algae material or dried material according to any method for preparing alginates known to a skilled person.

Cross-linked polymers as defined herein, used for preparation of the (spherical) core of the herein defined (spherical) microcapsule and cross-linked polymers, used for preparation of the at least one surface coating layer of the (spherical) microcapsule may be identical or different with respect to the selected polymer and with respect to the chosen concentrations.

According to a first embodiment the cross-linked polymers used for preparation of the (spherical) core and the at least one surface coating layer may comprise chemically identical polymers in identical or differing concentrations. Preferably, the polymers present in the (spherical) core and the at least one surface coating layer are prepared using a non-cross-linked polymer solution selected from any of the polymers as defined herein. In this polymer solution, the non-cross-linked polymers are typically present in a concentration of about 0.1% (w/v) to about 8% (w/v) of the non-cross-linked polymer, more preferably in a concentration of about 0.1% (w/v) to about 4% (w/v) of the non-cross-linked polymer, even more preferably in a concentration of about 0.5% (w/v) to about 2.5% (w/v) of the non-cross-linked polymer and most preferably in a concentration of about 1% (w/v) to about 2% (w/v) of the non-cross-linked polymer. If alginates as disclosed herein are used as polymers for the preparation of the (spherical) core of the (spherical) microcapsule as used herein and/or are used for preparation of the at least one surface coating of the (spherical) microcapsule, the concentration of the polymer solution for preparing the (spherical) core and the concentration of the polymer solution for preparing the at least one surface coating layer of the (spherical) microcapsule, may be selected independently upon each other from a concentration of 0.1 to 4% (w/v) of the non-cross-linked polymer, preferably from a concentration of 0.4 to 2% (w/v) of the non-cross-linked polymer. The alginate concentration for both solutions may be identical. Alternatively, different alginate concentrations may be used for preparing the (spherical) core and the at least one surface coating layer of the (spherical) microcapsules used according to the present invention. Preferably, the non-cross-linked polymers used for preparation of the (spherical) core and/or the at least one surface coating layer comprise chemically identical polymers, more preferably in identical concentrations, e.g. in concentrations as defined herein with polymers as defined herein. In this context the term “% (w/v)” refers to the concentration of non-cross-linked polymers and is typically determined on the basis of a certain amount of a polymer in its dry form versus the total volume of the polymer solution, e.g. after solubilising the non-cross-linked polymer in a suitable solvent (before the cross-linkage). However, the herein concentrations may instead also be meant to correspond to “% v/v” concentrations, if applicable, e.g. if polymers are used, which are present in a fluid aggregate state at standard conditions (room temperature, normal pressure, etc.).

According to a second embodiment the cross-linked polymers used for preparation of the (spherical) core and the at least one surface coating layer may comprise chemically different polymers in identical or differing concentrations. Thereby, concentrations and polymers may be chosen separately as defined herein for the (spherical) core and the at least one surface coating layer independent upon each other. Furthermore, polymers may be chosen from polymers as defined herein, including e.g. natural polymers, synthetic polymers, and combination of polymers, e.g. block polymers. The difference in the nature of the polymers used for the core or the at least one surface coating layer may also be due to different molecular weight of the polymers used and/or due to different cross-linkage of identical polymers, etc.

In case the (spherical) microcapsules comprise more than one surface coating layer, the polymers in each of the at least one surface coating layers may be identical or different, i.e. the cross-linked polymers of each surface coating layer may comprise chemically identical or different polymers in identical or differing concentrations. According to one example, the (spherical) microcapsule, as used according to the present invention, may comprise at least one surface coating layer, as defined herein, consisting of any polymer as defined herein, and an additional external surface coating layer consisting of polycations, e.g. polyamino acids as defined herein, e.g. poly-L-lysine, poly-L-glutamic acid, etc. Likewise, the difference in the nature of the polymers used for the differing surface coating layers may be due to a different molecular weight of the polymers used and/or due to different cross-linkage of identical polymers, etc.

The (spherical) core of the (spherical) microcapsule as used herein additionally comprises cells. Such cells are typically selected from stem cells or stromal cells, preferably mesenchymal stem cells or mesenchymal stromal cells, or may be selected from any other cell (type), that may be used in the context of the present invention, for treatment of a vascular disease or diseases related thereto. Such cells are typically obtainable by stably transfecting a cell with a nucleic acid or rather a vector containing at least one nucleic acid encoding at least GLP-1, or a fragment or variant thereof, as defined herein, and preferably additionally secreting VEGF.

Cells suitable for the (spherical) core of the (spherical) microcapsule as used herein may be chosen from (non-differentiated) stem cells including totipotent, pluripotent, or multipotent stem cells. Stem cells used in the present context preferably comprise embryonic stem cells or stem cells derived from the ectoderm, the mesoderm or the endoderm, or adult stem cells such as (human) mesenchymal stem cells or mesenchymal stromal cells (MSC, hMSC) (e.g. derived from human bone marrow or from fat tissue), hematopoietic stem cells, epidermal stem cells, neural stem cells and immature fibroblasts, including fibroblasts from the skin (myofibroblasts), etc. These (undifferentiated) stem cells are typically capable of symmetric stem cell division, i.e. cell division leading to identical copies. Stem cells maintain the capacity of transforming into any cell type. Moreover, stem cells are capable of dividing asymmetrically leading to a copy of the stem cell and another cell different from the stem cell copy, e.g., a differentiated cell. Once encapsulated, the cells typically do not divide anymore.

Stem cells as defined herein, particularly mesenchymal stem cells or mesenchymal stromal cells, suitable for the (spherical) core of the (spherical) microcapsule as used herein may additionally produce a set of endogenous trophic factors that support the cytoprotective effect of GLP-1 or of a fragment or variant thereof. Biologically active factors for this paracrine cytoprotective mechanism of the mesenchymal stromal cells may be e.g. the cytokines GRO, IL-6, IL-8, MCP-1 and the growth factors VEGF, GDNF and Neurotrophin-3. According to a particularly preferred embodiment, the cells in the (spherical) core of the (spherical) microcapsules therefore additional to VEGF may secrete endogenous proteins or peptides as paracrine factors that are released through the capsule in therapeutic levels selected from IL6, IL8, GDNF, NT3, and MCP1, etc.

The core of (spherical) microcapsule as used herein, may alternatively contain cells which are chosen from (differentiated) cells, e.g., obtainable from the herein described stem cells or stromal cells, e.g., cells of the connective tissue family, e.g., (mature) fibroblasts, cartilage cells (chondrocytes), bone cells (osteoblasts/osteocytes, osteoclasts), fat cells (adipocytes), or smooth muscle cells, or blood cells including lymphoid progenitor cells or cells derived therefrom, e.g., NK cells, T-cells, B-cells or dendritic cells, or common myeloid progenitor cells or cells derived therefrom, e.g., dendritic cells, monocytes, macrophages, osteoclasts, neutrophils, eosinophils, basophils, platelets, megakaryocytes or erythrocytes, or macrophages, neuronal cells including astrocytes, oligodendrocytes, etc., or epithelial cells, or epidermal cells. These differentiated cells, prior to encapsulation, are typically capable of symmetric cell division, i.e. cell division leading to identical copies of the differentiated parent cell. Moreover, in some cases these differentiated cells may be capable of dividing asymmetrically leading to an identical copy of the parent cell and another cell different from the parent cell, i.e. a cell being further differentiated than the parent cell. Alternatively, in some cases differentiated cells as defined herein may be capable of differentiating further without the need of cell division, e.g., by adding selective differentiation factors.

Furthermore, cells embedded in the (spherical) core of the (spherical) microcapsule, as used according to the present invention, may be cells taken from the patient (autologous cells) to be treated himself or may be taken from allogenic cells (e.g. taken from an established cell line cultivated in vitro, e.g., HEK293 cells, hTERT-MSC cells, etc.). Due to the surface coating layer embedding the (spherical) core in the (spherical) microcapsule, as used according to the present invention, it allows the use of allogenic cells without evoking any undesired immune response by the patient to be treated.

Cells embedded in the (spherical) core of the (spherical) microcapsule used according to the present invention, may furthermore be a combination of (differentiated and/or non-differentiated) cell types as defined herein. The (spherical) core of the (spherical) microcapsule, as used according to the present invention, may contain, e.g., human mesenchymal stem cells or human mesenchymal stromal cells, wherein a portion of these cells may be differentiated in vitro or in vivo into a cell type, such as defined herein, e.g. adipocytes (suitable for transplantation into fat tissue), etc. Accordingly, various cell types (derived e.g. from a specific stem cell type) may be allocated in the core, e.g. sharing a common lineage.

In summary, cells suitable for preparing the (spherical) core of the (spherical) microcapsule used according to the present invention may be selected from non-differentiated or differentiated cells. According to one embodiment non-differentiated cells as defined herein may be preferred. Such non-differentiated cells may provide advantageous properties, e.g. a prolonged effect of the (spherical) microcapsules used according to the present invention, e.g. the prolonged capability to express and secrete a GLP-1 peptide or a GLP-1 fusion peptide as defined herein, or a fragment or variant thereof, e.g. due to a longer life span of such non-differentiated cells. In an alternative embodiment, differentiated cells as defined herein may be preferred for preparing the (spherical) core of the (spherical) microcapsule used according to the present invention, since they typically do not proliferate any more and, thus, do not lead to any undesired proliferation of cells within the (spherical) core of the (spherical) microcapsule, as used according to the present invention. Specific differentiation of cells may be carried out by a skilled person in vitro according to methods known in the art by adding selected differentiation factors to precursor cells. Preferably, cells are differentiated in such a way that a vast majority of cells (or at least 90%, more preferably at least 95% and most preferably at least 99%) embedded in the (spherical) core of the (spherical) microcapsule used according to the present invention, belongs to the same cell type. In particular, mesenchymal stem cells as defined herein may be differentiated in vitro, e.g., into osteoblasts, chondrocytes, adipocytes such as fat cells, neuron-like cells such as brain cells, etc., and used herein accordingly. As to whether non-differentiated or differentiated cells are used for preparing the (spherical) core of the (spherical) microcapsule, as defined herein, may be dependent on specific requirements of the disease to be treated, e.g. the site of affliction, the administration mode, the tissue chosen for implant, etc. A selection of appropriate cells may be carried out by a skilled person evaluating these criteria.

Furthermore, cells suitable for preparing the (spherical) core of the (spherical) microcapsule as defined herein may be immortalised or non-immortalised cells, preferably immortalised cells. If immortalised cells are used, these cells preferably retain their capability of symmetric and/or asymmetric cell division as discussed herein. According to the present invention cells are defined as immortal when they exceed the double life span of normal cells (i.e. of non-immortalised cells). The maximum life span of normal diploid cells in vitro varies dependent on the cell type (e.g. foetal versus adult cell) and culture conditions. Thus, the maximum life span of cultured normal cells in vitro is approximately 60-80 population doublings. For example, keratinocytes may divide around 80 times, fibroblasts more than 50 times, and lymphocytes about 20 times. Normal bone marrow stromal cells may exhibit a maximum life span of 30-40 population doublings. Preferably, a cell line used for preparation of the (spherical) core of an (spherical) microcapsule, as used according to the present invention, may continuously grow past 350 population doublings and may still maintain a normal growth rate characteristic of young cells prior to encapsulation.

Methods for immortalising cells for preparing the (spherical) core of the inventive (spherical) microcapsule as defined herein are widely known in the art and may be applied here accordingly (see e.g. WO 03/010305 or WO 98/66827, which are incorporated herein by reference). An exemplary method (according to WO 03/010305) comprises e.g. following steps:
a) culturing cells, e.g., stem cells, in particular stem cells derived from human bone marrow (e.g. (human) mesenchymal stem cells (MSC, hMSC)), in accordance with standard conventional cell culturing methods known to the skilled person;
b) transducing said cell cultures with a retroviral vector, comprising at least a fragment of the human telomerase reverse transcriptase (hTERT) gene or a variant thereof, by
   b1) culturing a packaging cell line (e.g. PA317 cells, PG13 cells, Phenix, etc.), wherein the packaging cell line are cells in which the retroviral vector is produced,
   b2) constructing a retroviral vector (e.g. derived from Moloney murine leukaemia virus, etc.), wherein the retroviral vector comprises at least a fragment of the catalytic subunit of the human telomeric repeat (hTRT) gene or a variant thereof, more preferably a hTERT cDNA fragment, e.g. a 3452 base pair EcoRI fragment from pGRN145 (Geron Corporation),
   b3) transfecting said packaging cell line, with said retroviral vector,
   b4) transducing said packaging cell line with said transfected cells, preferably by centrifuging the cells with the retroviral vector,
   b5) transducing cultured cells according to step a) herein with the packaging cells of step b4), said cells comprising said retroviral vector.
c) obtaining an immortal cell line, wherein said immortalised cell line has substantially identical characteristics and properties compared to the cells of step a).

As a result the inserted polynucleotide sequence derived from the human telomeric subunit (hTRT) gene may be transcribed and translated to produce a functional telomerase. One of skill will recognize that due to codon degeneracy a number of polynucleotide sequences will encode the same telomerase. In addition, telomerase variants are included, which have sequences substantially identical to a wildtype telomerase sequence and retain the function of the wildtype telomerase polypeptide (e.g. resulting from conservative substitutions of amino acids in the wildtype telomerase polypeptide).

Cells embedded in the (spherical) core of the (spherical) microcapsule encoding and secreting at least GLP-1, or a fragment or variant thereof as defined herein, and preferably additionally secreting VEGF, may be further modified or engineered to additionally secrete a factor selected from the group consisting of anti-apoptotic factors, growth factors, erythropoietin (EPO), anti-platelet factors, anti-coagulant factors, anti-thrombotic drugs, anti-angiogenic factors, or any further factor exhibiting cardioprotective function, etc.

According to one specific embodiment, the cells embedded in the core of the (spherical) microcapsule encoding and secreting at least GLP-1, or a fragment or variant thereof as defined herein, and preferably additionally secreting VEGF. In this context, the cells typically already secrete VEGF and have been engineered to additionally secrete GLP-1 or a fragment or variant thereof as defined herein. The cells ydditionally may be engineered to additionally secrete erythropoietin (EPO). Erythropoietin (also known as EPO, epoetin or procrit) is an acidic glycoprotein hormone of approximately 34,000 dalton molecular weight occurring in multiple forms, including alpha, beta, omega and asialo. Erythropoietin stimulates red blood cell production. It is produced in the kidney and stimulates the division and differentiation of committed erythroid precursors in the bone marrow and elsewhere. Generally, erythropoietin is present in very low concentrations in plasma when the body is in a healthy state, in which tissues receive sufficient oxygenation from the existing number of erythrocytes. This normal low concentration is enough to stimulate replacement of red blood cells that are lost normally through aging. The amount of erythropoietin in the circulation is increased under conditions such as hypoxia, when oxygen transport by blood cells in the circulation is reduced. Hypoxia may be caused by loss of large amounts of blood through haemorrhage, destruction of red blood cells by over-exposure to radiation, reduction in oxygen intake due to high altitudes or prolonged unconsciousness, or various forms of anaemia or ischemia. In response to tissues undergoing hypoxic stress, erythropoietin will increase red blood cell production by stimulating the conversion of primitive precursor cells in the bone marrow into proerythroblasts which subsequently mature, synthesize haemoglobin and are released into the circulation as red blood cells. When the number of red blood cells in circulation is greater than needed for normal tissue oxygen requirements, erythropoietin in circulation is decreased. Preferably, erythropoietin is used as an additional factor contained in the cells to induce production of red blood cells to combat anaemia. (See, e.g., Bottomley et al. (2002) Lancet Oncol. 3:145). Erythropoietin has also been suggested to be useful in controlling bleeding in patients with abnormal haemostasis. (See e.g., U.S. Pat. No. 6,274,158). Recombinant human erythropoietin (rHuEpo or epoetin [alpha]) is commercially available as EPOGEN® (epoetin alfa, recombinant human erythropoietin) (Amgen Inc., Thousand Oaks, Calif.) and as PROCRIT® (epoetin alfa, recombinant human erythropoietin) (Ortho Biotech Inc., Raritan, N.J.). EPO may increase the hematocrit values in patients suffering from a vascular disease. The normal ranges for hematocrit values of erythropoietin are 37-48 percent for women and 42-52 percent for men (see Case Records of the Massachusetts General Hospital: normal reference laboratory values. (1992) N. Eng. J. Med. 327:718). Of course, the safety and efficacy of use of erythropoietin to increase hematocrit levels in patients with cardiovascular disease, especially those suffering from renal failure, must be further evaluated. Preferably, erythropoietin is typically provided at a concentration or for a duration that will not induce red blood cell formation or alternatively, increase the hematocrit in a subject, e.g., between about 1 μM and less than 1000 μLEM, including less than 900 μM, less than 700 μM, less than 500 μM, less than 300 μM, less than 100 μM, or less than 50 μM. In other embodiments, erythropoietin is administered as a function of the subject's body weight. Erythropoietin may typically be provided at a concentration of between about 1 U/kg to 10,000 U/kg of a subject's body weight, including less than 7,500 U/kg, 5,000 U/kg, 2500 U/kg, 1000 U/kg, 750 U/kg, 500 U/kg, 250 Ug/kg, 100 Ug/kg, 50 U/kg, 25 U/kg, 10 U/kg, 5 U/kg, or 1 U/kg. In this context, erythropoietin serum concentration is normally within the range of 5-50 mU/ml. For patients suffering from MI or AMI or other conditions associated thereto, erythropoietin is preferably provided either at a concentration of 50-100 U/kg depending on symptom, body weight, sex, animal species and the like. It is generally assumed that treatment options holding the blood concentration at about 1-100 mU/ml will be preferred. Also preferably, erythropoietin is typically provided at a concentration that does not increase the hematocrit in a survivor, wherein the erythropoietin is administered in a single dose within 1, 2 or 3 hours of the myocardial infarction, for an extended period of time.

According to one further specific embodiment, the cells embedded in the core of the (spherical) microcapsule encoding and secreting at least GLP-1, or a fragment or variant thereof as defined herein preferably additionally secrete VEGF.

According to another specific embodiment, the cells embedded in the core of the (spherical) microcapsule encoding and secreting at least GLP-1, or a fragment or variant thereof as defined herein, and preferably secrete VEGF, may be engineered to additionally secrete antiapoptotic factors. Such factors may include, without being limited thereto, APC (apoptosis repressor with caspase recruitment domain), Bcl-2, Bcl-xL, Che-1/AATF, clusterin, insulin, Mcl-1, NF-kB-dependent anti-apoptotic factors, serotonin, survivin, etc. Furthermore, any factor, which acts as an inhibitory factor to an apoptotic factor known in the art, and which may thus be regarded as antiapoptotic factors, is encompassed herewith. Such factors are preferably encoded by a nucleic acid and secreted by the cells encoding and secreting the GLP-1 peptides and GLP-1 fusion peptides as defined herein. In this context, such antiapoptotic factors may be directed against at least one of the following apoptotic factors or apoptosis related proteins including AIF, Apaf e.g. Apaf-1, Apaf-2, Apaf-3, oder APO-2 (L), APO-3 (L), Apopain, Bad, Bak, Bax, Bcl-2, Bcl-x_{L}, Bcl-x_{S}, bik, CAD, Calpain, Caspase e.g. Caspase-1, Caspase-2, Caspase-3, Caspase-4, Caspase-5, Caspase-6, Caspase-7, Caspase-8, Caspase-9, Caspase-10, Caspase-11, ced-3, ced-9, c-Jun, c-Myc, crm A, cytochrom C, CdR1, DcR1, DD, DED, DISC, DNA-PK_{CS}, DR3, DR4, DR5, FADD/MORT-1, FAK, Fas (Fas-ligand CD95/fas (receptor)), FLICE/MACH, FLIP, fodrin, fos, G-Actin, Gas-2, gelsolin, granzyme A/B, ICAD, ICE, JNK, lamin A/B, MAP, MCL-1, Mdm-2, MEKK-1, MORT-1, NEDD, NF-ₖₐₚₚₐB, NuMa, p53, PAK-2, PARP, perforin, PITSLRE, PKCdelta, pRb, presenilin, prICE, RAIDD, Ras, RIP, sphingomyelinase, thymidinkinase from herpes simplex, TRADD, TRAF2, TRAIL-R1, TRAIL-R2, TRAIL-R3, transglutaminase, etc.

A GLP-1 peptide encoded and secreted by a cell contained in the (spherical) core of the (spherical) microcapsule, as defined herein, may be selected from any known GLP-1 peptide sequence or from any known GLP-1 fusion peptide sequence. In this context, the neuroprotective factor GLP-1 is located on the well studied glucagon gene, which encodes preproglucagon (see e.g. White, J. W. et al., 1986 Nucleic Acid Res. 14(12) 4719-4730). The preproglucagon molecule as a high molecular weight precursor molecule is synthesized in pancreatic alpha cells and in the jejunum and colon L cells. Preproglucagon is a 180 amino acid long prohormone and its sequence contains, in addition to glucagon, two sequences of related structure: glucagon-like peptide-1 (GLP-1) and glucagon-like peptide-2 (GLP-2). In the preproglucagon molecule, between GLP-1 and GLP-2 is a 17 amino acid peptide sequence (or rather a 15 amino acid sequence plus the C-terminal RR cleavage site), intervening peptide 2 (IP2). The IP2 sequence (located between GLP-1 and GLP-2 in the precursor molecule) is normally cleaved proteolytically after aa 37 of GLP-1 in vivo. The preproglucagon module is therefore cleaved into various peptides, depending on the cell, and the environment, including GLP-1 (1-37), a 37 amino acid peptide in its unprocessed form. Generally, this processing occurs in the pancreas and the intestine. The GLP-1 (1-37) sequence can be further proteolytically processed into active GLP-1 (7-37), the 31 amino acid processed form, or its further degeneration product GLP-1 (7-36) amide. Accordingly, the designation GLP-1(7-37) means that the fragment in question comprises the amino acid residues (starting) from (and including) number 7 to (and including) number 37 when counted from the N-terminal end of the parent peptide, GLP-1. The amino acid sequence of GLP-1(7-36), GLP-1(7-36)amide and of GLP-1(7-37) is given in formula I (SEQ ID NO: 25):

(I)
His-Ala-Glu-Gly-Thr-Phe-Thr-Ser-Asp-Val-Ser-Ser-Tyr-Leu-Glu-Gly-Gln-Ala-Ala-Lys-
Glu-Phe-Ile-Ala-Trp-Leu-Val-Lys-Gly-Arg-X

which shows GLP-1(7-36)amide when X is NH₂ or GLP-1(7-36), when X is absent, and GLP-1(7-37) when X is Gly-OH.

According to one embodiment of the present invention, the GLP-1 peptide may therefore be selected from any known GLP-1 peptide sequence, e.g. as defined herein. In this context, the GLP-1 peptide may be secreted by cells embedded in the (spherical) core of the (spherical) microcapsule which thus may be transfected preferably prior to preparing the (spherical) core with nucleic acid sequences encoding a GLP-1 peptide as defined herein such that these cells express and secrete the GLP-1 peptide. Preferably a GLP-1 peptide as used herein, which may be encoded and secreted by a cell embedded in the (spherical) microcapsule, may be selected from a group consisting of a peptide comprising aa 7-35 of (wt) GLP-1 or a peptide showing an identity of at least 80%, 90%, 95% or even 99% with this peptide. In general, the GLP-1 peptide may be selected from group consisting of (i) a peptide comprising aa 1-37 of (wt) GLP-1, (ii) a peptide comprising aa 7-35, 36 or 37 of (wt) GLP-1, (iii) GLP-1(7-36)amide and (iv) a peptide showing an identity of at least 80%, 90%, 95% or even 99% with any of these peptides, including modified peptides. In this context, a “modified GLP-1 peptide” is intended to mean any GLP-1 variant or a GLP-1 fragment, including combinations, e.g. a fragment of a variant, which retain the biological function of (wt) GLP-1. Variants and fragments are categorized as modifications of the unmodified GLP-1 sequence, e.g. GLP-1(7-35, 36 or 37). Within the meaning of the present invention any variant or fragment has to be functional, e.g. has to exert the same or a similar biological activity as the unmodified (GLP-1) peptide. The term “activity” refers to the biological activity (e.g. one or more of the biological activities comprising receptor binding, activation of the receptor, exhibition of beneficial effects known for GLP-1, e.g. its activity to powerfully reduce the damages caused by ischemia or oxygen shortage and potential death of heart tissue as mentioned herein in connection with the effects of GLP-1 as described in the prior art, which may be compared under the same conditions for the naturally occurring GLP-1 peptide as defined herein and any fragment or variant thereof. Preferably, a variant or fragment of a GLP-1 peptide as defined herein exerts at least 25% activity of a GLP-1(7-35, 36 or 37), more preferably at least 50% (biological) activity, even more preferably 60, 70, 80 or 90% (biological) activity and most preferably at least 95 or 99% (biological) activity of a GLP-1(7-35, 36 or 37) as defined herein. The biological activity may be determined by a standard assay, e.g. which preferably allows determining the activity as an incretin hormone lowering the blood glucose level, e.g. using an animal model for diabetes type 2, etc.

According to a particularly preferred embodiment, the GLP-1 peptide or a GLP-fusion peptide as defined herein, which may be as encoded by cells embedded in the (spherical) core of the (spherical) microcapsule, does not include at its N-terminus the naturally occurring amino acids 1 to 6 of a (native) GLP-1 (1-37) sequence as defined herein. Even more preferably, the GLP-1 peptide as defined herein or a GLP-fusion peptide as defined below does not include at its N-terminus the naturally occurring amino acids 1, 2, 3, 4, 5 and/or 6 of a native GLP-1 (1-37) sequence as defined herein. This proviso preferably refers to GLP-1 peptides as defined herein, e.g. selected from the group consisting of a peptide comprising aa 7-35, 36 or 37 of GLP-1, GLP-1(7-36)amide and a peptide showing an identity of at least 80%, 90%, 95% or even 99% with any of these peptides, including modified peptides, and to GLP-1 fusion peptides containing such GLP-1 peptides. However, this proviso does not exclude, that such a GLP-1 peptide as defined herein or a GLP-1 fusion peptide as defined herein, comprises an N-terminal (and/or C-terminal) sequence modification or additional amino acids or peptides fused thereto, e.g. signal peptide sequences and/or leader peptide sequences, etc., however being distinct from the sequence of amino acids 1 to 6 of wt GLP-1. In another preferred embodiment, any amino acid attached to the N-terminus of GLP-1 (7-35, 36 or 37) of homologs thereof does not correspond to the naturally occurring amino acid at position 6 of GLP-1(7-35, 36 or 37). According to a further preferred embodiment, any amino acid (directly) attached to the N-terminus of GLP-1 (7-35, 36 or 37) of homologs thereof does not correspond to the naturally occurring amino acid 6, to the naturally occurring amino acids 5 and 6, to the naturally occurring amino acids 4, 5 and 6, to the naturally occurring amino acids 3, 4, 5, and 6, to the naturally occurring amino acids 2, 3, 4, 5, and 6 or to the naturally occurring amino acids 1, 2, 3, 4, 5, and 6 of native GLP-1, preferably in their native order in GLP-1. According to a particularly preferred embodiment, any amino acid attached to the N-terminus of GLP-1 (7-35, 36 or 37) of homologs thereof does not correspond to the sequence of preproglucagon.

Native GLP-1, particularly GLP-1 (7-36), suffers from a short half life in vivo and therefore is of limited use in therapeutic treatments in general, where a frequent administration is strictly to be avoided or where a long-term administration is envisaged. GLP-1 is rapidly degraded in plasma within minutes by DPP-IV (dipeptidyl peptidase IV) between residues 8 and 9, resulting in an inactive NH₂-terminally truncated metabolite GLP-1 (9-36). Additionally, native GLP-1 typically undergoes renal excretion. These factors raise the issue, as to which peptide, GLP-1 (7-36) or the NIL-terminally truncated metabolite GLP-1 (9-36), is the active moiety in vivo and as to whether physiological effects are exerted in therapeutic applications by the native GLP-1 or its fragments. As a consequence and due to its rapid degradation in vivo, native GLP-1 or its fragments may be used as a suitable tool for a short-term metabolic control, such as intensive care units potentially useful in patients suffering from an acute vascular disease or diseases related thereto.

To avoid such fast degradation, various attempts have been made to synthesize stabilized (against degradation by DPP-IV) analogues of naturally occurring GLP-1 (e.g. GLP-1(7-37)). In particular, the 8^{th} residue, which in vivo is Ala, was replaced by another residue, for instance, Gly, Ser or Thr (Burcelin, R. et al. (1999) Metabolism 48, 252-258). The Gly8 (or G8) analogue has been extensively tested, both as synthesized molecule, and produced by cell lines genetically engineered to secrete the mutant polypeptide (Burcelin, R., et al. (1999), Annals of the New York Academy of Sciences 875: 277-285). Various other modifications have been introduced into e.g. GLP-1(7-37) to enhance its in vivo stability without compromising its biological activity.

Such an approach circumvents the problem of short half life by stabilization of GLP-1 against degradation by DPP-IV, e.g. by additionally administering a DPP-IV inhibitor with the GLP-1 peptide. Additionally administering a DPP-IV inhibitor with the GLP-1 peptide is complicated and typically does not lead to the desired long-term treatment as the DPP-IV inhibitor may only be used efficiently in in vitro systems.

Therefore, according to an alternative embodiment, a GLP-1 peptide encoded and secreted by cells embedded in the core of the (spherical) microcapsule may be selected from a GLP-1 fusion peptide or a variant or fragment thereof. The GLP-1 fusion peptide as used herein may be encoded and secreted by cells embedded in the (spherical) core of the (spherical) microcapsule as defined herein. In this context, cells embedded in the (spherical) core of the (spherical) microcapsule, as defined herein, are typically transfected prior to preparing the core with nucleic acid sequences encoding the GLP-1 fusion peptide such that these cells encode, express and secrete the GLP-1 fusion peptide.

The GLP-1 fusion peptides as defined herein preferably have at least two components, e.g. components (I) and (II), components (I) and (III) or components (I), (II) and (III), exhibit GLP-1's biological activity as defined herein and, simultaneously, confer stability to component (I) of GLP-1 fusion peptides typically by (such) a C-terminal elongation. Component (I) of GLP-1 fusion peptides as defined herein typically contains a sequence of a GLP-1 peptide as defined herein, preferably a sequence having at least 80%, more preferably at least 85% and even more preferably at least 90% sequence identity with SEQ ID NO: 1. SEQ ID NO: 1 represents the native amino acid sequence of GLP-1(7-37) (length of 31 amino acids), which is strictly conserved among mammalians. According to a particularly preferred embodiment, component (I) of GLP-1 fusion peptides as defined herein contains a sequence being identical to SEQ ID NO: 1 or a sequence, which lacks amino acids 36 and/or 37 of SEQ ID NO: 1.

Component (II) of the GLP-1 fusion peptide, which may be encoded and secreted by cells embedded in the (spherical) core of the (spherical) microcapsule as defined herein, (or more generally any GLP-1 peptide including fragments or variants of fusion peptides) typically contains a peptide sequence having at least nine amino acids. The GLP-1 fusion peptide may typically have in its component (II) a sequence length of 9 to 30, preferably 9 to 20, and most preferably 9 to 15 amino acids. Generally spoken, shorter sequences in component (II) may be preferred due to their superior binding activity to the GLP receptor over longer sequences. The sequence of component (II), even though it is not a prerequisite, may preferably be neutral or may have a negative charge at pH 7. Component (II) of the GLP-1 fusion peptide furthermore may contain at least one proline residue in its sequence. Proline residues are common amino acids within a β-turn forming tetrameric amino acid sequence. Thus, component (II) of the GLP-1 fusion peptide may form a β-turn like structure. A β-turn structure is a typical secondary structure element of proteins or peptides. It is typically formed by a stretch of four amino acids, which reverts the direction of the peptide's or protein's backbone chain direction. If present in the GLP-1 fusion peptide, the proline residue is commonly located at position 2 or 3, preferably at position 2, of a tetrameric β-turn sequence motif occurring in component (II) of the GLP-1 fusion peptide.

Component (II) of the GLP-1 fusion peptide, which may be encoded and secreted by cells embedded in the (spherical) core of the (spherical) microcapsule as defined herein, (or more generally any GLP-1 peptide including fragments or variants of fusion peptides) may contain a sequence motif selected from the group consisting of VAIA, IAEE, PEEV, AEEV, EELG, AAAA, AAVA, AALG, DFPE, AADX, AXDX, and XADX, wherein X represents any amino acid (naturally occurring or a modified non-natural amino acid). These tetrameric motifs may be located anywhere in the sequence of component (II). In a particularly preferred embodiment, the inventive fusion peptide component (II) is a peptide sequence being linked to the C-terminus of component (I) by its N-terminal sequence motif selected from the group consisting of AA, XA, AX, RR, RX, and XR, wherein X represents any amino acid (naturally occurring or a modified non-natural amino acid).

Particularly preferred as component (II) of a GLP-1 fusion peptide, which may be encoded and secreted by cells embedded in the (spherical) core of the (spherical) microcapsule as defined herein, is a peptide sequence containing a sequence according to SEQ ID NO: 48: X₁X₁DFPX₂X₂X₃X₄, corresponding to a partial sequence of human or murine IP-2, wherein each X₁ is typically selected independently upon each other from any naturally occurring amino acid, preferably arginine (R) or alanine (A), more preferably alanine (A), or may be absent; wherein each X₂ is typically selected independently upon each other from aspartic acid (D) or glutamic acid (E), and wherein each X₃ and X₄ is typically selected independently upon each other from any naturally occurring amino acid, preferably alanine (A), glycine (G), isoleucine (I), leucine (L), threonine (T), or valine (V). X₄ also may be absent.

Even more preferred as component (II) of a GLP-1 fusion peptide, which may be encoded and secreted by cells embedded in the (spherical) core of the (spherical) microcapsule as defined herein, is a peptide sequence containing a sequence according to SEQ ID NO: 22 (RRDFPEEVAI), SEQ ID NO: 27 (DFPEEVAI), SEQ ID NO: 28 (RDFPEEVA), or SEQ ID NO: 29 (RRDFPEEV), SEQ ID NO: 30 (AADFPEEVAI), SEQ ID NO: 31 (ADFPEEVA), or SEQ ID NO: 32 (AADFPEEV), (all peptide sequences given in the one-letter-code) or a sequence having at least 80% sequence identity with SEQ ID NO: 22, 27, 28, 29, 30, 31 or 32. SEQ ID NO: 22 is a partial sequence of the full-length IP-2 (intervening peptide 2) sequence, which contains the 10 N-terminal amino acids of the 15 amino acid long full-length IP-2 sequence. IP-2 is a preferred example of a component (II) as used herein. Accordingly, other stronger preferred sequences being contained in component (II) of the herein defined GLP-1 fusion peptide are longer partial amino acid sequences of IP-2, such as the 14 N-terminal amino acid sequence occurring in humans (SEQ ID NO: 23 (RRDFPEEVAIVEEL)) or its murine counterpart (SEQ ID NO: 24 (RRDFPEEVAIAEEL)), or sequences (SEQ ID NO: 33 (AADFPEEVAIVEEL)) or (SEQ ID NO: 34 (AADFPEEVAIAEEL)), or a sequence having at least 80% sequence identity with SEQ ID NOs: 23, 24, 33 or 34. Most preferred as elements being contained in component (II) of the GLP-1 fusion peptide are full-length IP-2 sequences having all 15 amino acids of the naturally occurring IP-2 sequence (SEQ ID NO: 2 (RRDFPEEVAIVEELG), human, or SEQ ID NO: 3 (RRDFPEEVAIAEELG), murine, or SEQ ID NO: 35 (AADFPEEVAIVEELG), or SEQ ID NO: 36 (AADFPEEVAIAEELG)) or a sequence having at least 80% sequence identity with SEQ ID NOs: 2, 3, 35 or 36. Within the scope of the present invention are also all mammalian isoforms of IP2 (natural variants of IP2 among mammalians). More than one copy of a sequence being included into component (II) may be provided, e.g. 2, 3 or even more copies of IP2 or a fragment or variant of IP2.

Accordingly, a GLP-1 fusion peptide, encoded and secreted by cells embedded in the (spherical) core of the (spherical) microcapsule, as defined herein, preferably contains, comprises or consists of sequences according to SEQ ID NO: 8 (HAEGTFTSDVSSYLEGQAAKEFIAWLVKGRGRRDFPEEVAIAEELG), i.e. GLP-1(7-37) linked without any linker sequence via its C-terminus to murine IP2 or according to SEQ ID NO: 12 (HAEGTFTSDVSSYLEGQAAKEFIAWLVKGRGRRDFPEEVAIVEELG), i.e. GLP-1(7-37) linked without any linker sequence via its C-terminus to human IP2, or sequences according to SEQ ID NO: 37 (HAEGTFTSDVSSYLEGQAAKEFIAWLVKGRGAADFPEEVAIAEELG), i.e. GLP-1(7-37) linked without any linker sequence via its C-terminus to IP2 or according to SEQ ID NO: 38 (HAEGTFTSDVSSYLEGQAAKEFIAWLVKGRGAADFPEEVAIVEELG), i.e. GLP-1(7-37) linked without any linker sequence via its C-terminus to IP2, or a sequence SEQ ID NO: 39 (HAEGTFTSDVSSYLEGQAAKEFIAWLVKGRGRRDFAEEVAIAEELG), SEQ ID NO: 40 (HAEGTFTSDVSSYLEGQAAKEFIAWLVKGRGRRDAAAAVAIAEELG), SEQ ID NO: 41 (HAEGTFTSDVSSYLEGQAAKEFIAWLVKGRGAADAAAAVAIAAALG), SEQ ID NO.: 42 (HAEGTFTSDVSSYLEGQAAKEFIAWLVKGRGRRDFP), SEQ ID NO: 43 (HAEGTFTSDVSSYLEGQAAKEFIAWLVKGRGRRDFPEEVA), SEQ ID NO: 44 (HAEGTFTSDVSSYLEGQAAKEFIAWLVKGRGRRDFPEEVAIAEELGRRHAC), SEQ ID NO: 45 (HAEGTFTSDVSSYLEGQAAKEFIAWLVKGRGRRDFAEEVAIVEELG), SEQ ID NO: 46 (HAEGTFTSDVSSYLEGQAAKEFIAWLVKGRGRRDAAAAVAIVEELG), SEQ ID NO: 47 (HAEGTFTSDVSSYLEGQAAKEFIAWLVKGRGAADAAAAVAIVAALG), or SEQ ID NO: 48 (HAEGTFTSDVSSYLEGQAAKEFIAWLVKGRGRRDFPEEVAIVEELGRRHAC), i.e. GLP-1(7-37) linked without any linker sequence via its C-terminus to specific analogs or variants of the IP2 sequence. Variants or fragments thereof having a sequence identity of at least 80% with SEQ ID NOs: 8, 12, and 37 to 48, or fragments or variants thereof may be used herein as well. Preferred GLP1-fusion peptides in this context may further comprise sequences according to SEQ ID NOs: 13, 14, 19 and 20.

Without being bound to any theory, it is concluded by the inventors of the present invention that the instability of GLP-1(7-35, 36 or 37), e.g. if secreted in vivo into the patients surrounding tissue by cells embedded in the (spherical) core of the implanted (spherical) microcapsule used according to the present invention, is due to its unprotected 3-dimensional structure. Proteases may cleave the GLP-1(7-35, 36 or 37) peptide and abolish its physiological activity rapidly in vivo. By linking a peptide sequence to the C-terminus of GLP-1(7-35, 36 or 37) its structure gains stability towards enzymatic degradation. Such gain in stability may be enhanced, if the additional C-terminal peptide sequence (being contained in component (II) of the fusion peptide according to the invention) folds back, e.g. due to the presence of a β-turn structural element formed by its primary structure and providing rigidity to component (II). The GLP-1 fusion peptide as defined herein, by virtue of its C-terminal peptide extension preferably containing a β-turn structural element, is found to have improved resistance to DPP-IV inactivation. The C-terminal peptide is either not cleaved from the GLP-1(7-35, 36 or 37) sequence prior to acting on its receptor in target cells or it may be cleaved enzymatically to form GLP-1(7-35, 36 or 37) in vivo. Irrespective of the exact form of the GLP-1 peptide bound at the site of the GLP-1 receptor, a GLP-1 peptide as defined herein exerts its function as an active neuroprotective compound. GLP-1 peptide sequences, which are considered to be suitable for component (II) of a GLP-1 fusion peptide as defined herein due to a primary structure forming a β-turn element, may readily be identified by adequate, e.g., spectroscopic methods, e.g. circular dichroism, or other methods known to the skilled person.

Component (II) and component (I) of a GLP-1 fusion peptide, which may be encoded and secreted by cells embedded in the (spherical) core of the (spherical) microcapsule as defined herein, may be directly linked or linked via a linker sequence. Preferably, both components are directly linked with each other. In case they are linked via a linker (or spacer), the linker is preferably a peptide linker. The peptide linker typically has a length of 1 to 10 amino acids, preferably 1 to 5, even more preferably 1 to 3 amino acids, in some cases the linker sequence may be even longer comprising 11 to 50 amino acids. The peptide linker may be composed of various (naturally occurring) amino acid sequences. Preferably, the peptide linker will introduce some structural flexibility between components to be linked. Structural flexibility is achieved e.g. by having a peptide linker containing various glycine or proline residues, preferably at least 30%, more preferably at least 40% and even more preferably at least 60% proline and glycine residues within the linker sequence. Irrespective of the specific sequence the peptide linker may preferably be immunologically inactive.

GLP-1 fusion peptides, which may be encoded and secreted by cells embedded in the (spherical) core of the (spherical) microcapsule as defined herein, may additionally contain a component (III). Generally, component (III) comprises at least four amino acid residues, preferably at least 10 additional amino acid residues, more preferably at least 20, or most preferably at least 30. In functional terms, component (III) is intended to further enhance the stability of a GLP-1 peptide as defined herein. Component (III) is expected not to interfere with the biological function of the GLP-1 fusion peptide, which is approximately comparable to the biological activity of GLP-1(7-37). Generally spoken, any C-terminal elongation of component (I) as defined herein, whether it is component (II), component (III) or a combination of components (II) and (III) as defined herein, enhances stability of component (I), i.e. a GLP-1 peptide as defined herein, e.g. GLP-1(7-35, 36 or 37), or its fragments or variants as defined herein.

Preferably, component (III) of the GLP-1 fusion peptide as defined herein, comprises at least 4, preferably at least 10, more preferably at least 20 additional amino acid residues of the N-terminal sequence of an isoform of GLP-2 of any mammalian organism (other naturally occurring variant of GLP-2 among mammalian), e.g. murine or human isoforms as shown in SEQ ID NOs: 4 and 5. GLP-2 occurs in pro-glucagon and is also involved in carbohydrate metabolism. In the context of the present invention, the term “GLP-2 peptide” preferably means GLP-2 (1-33, 34, or 35), whereas “modified GLP-2 peptide” is intended to mean any GLP-2 fragment or variant, or a fragment or variant of GLP-2(1-33, 34 or 35). Variants or fragments are categorized as modifications of the unmodified sequence, e.g. GLP-2(1-33, 34 or 35). As with the biologically active sequence included in component (I) (GLP-1 peptide), component (III) may also comprise variants or fragments of naturally occurring forms of GLP-2. Alternatively, component (III) may also comprise at least 4, preferably at least 10, more preferably at least 20 additional amino acid residues of the (N-terminal) sequence of GLP-1(7-37), correspondingly including all mammalian isoforms or—as disclosed herein—all functional fragments or variants thereof. Generally speaking, component (III) may contain any form of a GLP-1 peptide or a modified GLP-1 peptide, which is disclosed herein as suitable for component (I) of the GLP-1 fusion peptide. In a further alternative, component (III) may also contain chimeric forms of GLP-1(7-37) and GLP-2. A chimeric form may be produced by coupling GLP-1(7-37) and GLP-2 (or fragments or variants) with each other and by subsequently introducing this chimeric form as component (III) into the GLP-1 fusion peptide. Preferably, the chimeric form is composed of a partial sequence of GLP-1(7-37) and a partial sequence of GLP-2 linked together. E.g. the chimeric form may include the N-terminal 5 to 30 amino acids of GLP-1 and the C-terminal 5 to 30 amino acids of GLP-2 or vice versa, e.g. amino acids 7 or 8 to 22, 23, 24, 25, 26, 27, or 28 of GLP-1(7-37) and amino acid sequence from position 15, 16, 17, 18, 19, 20, 21, 22, 23 or 24 to e.g. the C-terminus of GLP-2. If modifications of naturally occurring forms of GLP-2 or GLP-1(7-37), respectively, are contained as component (III), component (III) preferably contains the sequence of SEQ ID NOs: 1, 4 or 5, respectively, or a sequence having at least 80% sequence identity with any of SEQ ID NOs: 1, 4 or 5.

In another embodiment, component (III) of the GLP-1 fusion peptide, which may be encoded and secreted by cells embedded in the (spherical) core of the (spherical) microcapsule as defined herein, may contain a plurality of sequences as described herein for components (I), (II) or (III). E.g. component (III) may contain at least two, preferably 2, 3, or 4 copies of GLP-1(7-37) and/or GLP-2 or at least two copies of sequences having at least 80% sequence identity with SEQ ID NOs: 1, 4 or 5. Also, component (III) may contain more than one copy of a chimeric version of GLP-1(7-37) or GLP-2, as disclosed herein, e.g. eventually forming a combination of chimeric version(s) together with GLP-1(7-37) and/or GLP-2 or its modifications with at least 80% sequence identity. A GLP-1 fusion peptide, which may be encoded and secreted by cells embedded in the (spherical) core of the (spherical) microcapsule as defined herein may also comprise two or more, preferably two, components (III), which may e.g. be (1) linked by its N-terminus to the C-terminus of component (I) or (II) and (2) linked by its C-terminus to the N-terminus of component (I) via a linker or directly. If two components (III) are provided, these may be identical or different.

According to a preferred embodiment, a GLP-1 fusion peptide, encoded and secreted by cells embedded in the (spherical) core of the (spherical) microcapsule as defined herein, may comprise the herein defined components (I), (II) and (III). Specific embodiments containing all of these components are preferably selected from a group consisting of SEQ ID NO: 6 (N-GLP-1(7-37)-IP2(murine)-RR-GLP-1(7-37)-C, also designated murine CM1 herein), SEQ ID NO: 7 (N-GLP-1(7-37)-IP2(murine)-RR-GLP2-C, also designated murine CM2 herein), SEQ ID NO: 10 (N-GLP-1(7-37)-IP2(human)-RR-GLP-1(7-37)-C, also designated human CM1), and SEQ ID NO: 11 (N-GLP-1(7-37)-IP2(human)-RR-GLP-2-C), also designated human CM2 herein) or a sequence having at least 80% sequence identity with SEQ ID NOs: 6, 7, 10, or 11 or a fragment or variant thereof. In the (directly) afore-mentioned sequences the terms “N” and “C” indicate N- and the C-terminus of these fusion peptides. All sequences according to SEQ ID NOs: 6, 7, 10 and 11 contain an RR-Linker (two arginine residues) at the C-terminus of IP2 (component (II)), which may alternatively also be discarded. Component (I) in each of the embodiments according to SEQ ID NOs: 6, 7, 10 or 11 is GLP-1(7-37), whereas component (III) (in each of these embodiments linked to the C-terminus of component (II)) is either GLP-1(7-37) or GLP-2. Preferred GLP1-fusion peptides in this context may further comprise sequences according to SEQ ID NOs: 15, 16, 17, 18 and 26.

In another preferred embodiment of the present invention, a GLP-1 fusion peptide, which may be encoded and secreted by cells embedded in the (spherical) core of the (spherical) microcapsule, as defined herein, contains in addition to component (I) a component (III) (without any component (II) as defined herein) which is either linked to the C-terminus of component (I) and/or to the N-terminus of component (I). Preferably, component (III) is located at the C-terminus of component (I). Irrespective of whether component (III) is linked to the N-terminus of component (I) (by its C-terminus) or to the C-terminus of component (I) (by its N-terminus), the coupling may be direct or indirect via a linker sequence. With regard to the linker sequence it is referred to the herein disclosure of GLP-1 fusion peptides for a linker connecting component (I) and component (II) of the GLP-1 fusion peptide.

In an alternative preferred embodiment of the present invention, a GLP-1 fusion peptide, which may be encoded and secreted by cells embedded in the (spherical) core of the (spherical) microcapsule, as defined herein, contains in addition to components (I) and (II) a component (III) which is either linked to the C-terminus of component (II) and/or to the N-terminus of component (I). Preferably, component (III) is located at the C-terminus of component (II). Irrespective of whether component (III) is linked to the N-terminus of component (I) (by its C-terminus) or to the C-terminus of component (II) (by its N-terminus), the coupling may be direct or indirect via a linker sequence.

With regard to the linker sequence it is again referred to the herein depicted disclosure of GLP-1 fusion peptides for a linker connecting component (I) and component (II) of the GLP-1 fusion peptide.

The GLP-1 fusion peptide, which may be encoded and secreted by cells embedded in the (spherical) core of the (spherical) microcapsule, as used according to the present invention, may furthermore comprise in addition to any of the afore mentioned combinations of components of the fusion protein (i.e. components (I) and (II), components (I) and (III) or components (I), (II) and (III)) a carrier protein, in particular transferrin or albumin, as component (IV). Such a component (IV) may be linked to the N- and/or C-terminus of any of the afore mentioned combinations of components of the GLP-1 fusion protein, i.e. components (I) and/or (II), components (I) and/or (III) or components (I), (II) and/or (III), either directly or using a linker as defined herein.

In a specific embodiment of the invention, the GLP-1 (fusion) peptide as defined herein, i.e. a GLP-1 peptide or a GLP-1 fusion peptide as defined above, which may be encoded and secreted by cells embedded in the (spherical) core of the (spherical) microcapsules as used herein, contains as component (I) and/or (III) a modified GLP-1 peptide comprising the amino acid sequence of the following formula II:

Xaa7-Xaa8-Glu-Gly-Thr-Phe-Thr-Ser-Asp-Xaa16-Ser-Xaa18-Xaa19-Xaa20-Glu-Xaa22-
Xaa23-Ala-Xaa25-Xaa26-Xaa27-Phe-Ile-Xaa30-Trp-Leu-Xaa33-Xaa34-Xaa35-Xaa36-
Xaa37,

wherein Xaa7 is L-histidine; Xaa8 is Ala, Gly, Val, Leu, Ile, or Lys, whereby Gly is particularly preferred; Xaa16 is Val or Leu; Xaa18 is Ser, Lys or Arg; Xaa19 is Tyr or Gln; Xaa20 is Leu or Met; Xaa22 is Gly or Glu; Xaa23 is Gln, Glu, Lys or Arg; Xaa25 is Ala or Val; Xaa26 is Lys, Glu or Arg; Xaa27 is Glu or Leu; Xaa30 is Ala, Glu or Arg; Xaa33 is Val or Lys; Xaa34 is Lys, Glu, Asn or Arg; Xaa35 is Gly; Xaa36 is Arg, Gly or Lys or amide or absent; Xaa37 is Gly, Ala, Glu, Pro, Lys, amide or is absent, wherein these amino acids are preferably selected if the GLP-1 (fusion) peptide as defined herein is encoded and secreted by cells embedded in the (spherical) core of the (spherical) microcapsules as used herein, to be administered to a patient in need thereof, when treating an when treating a vascular disease or diseases related thereto as defined herein,
or wherein Xaa7 is L-histidine, D-histidine, desamino-histidine, 2-amino-histidine, 3-hydroxy-histidine, homohistidine, N-acetyl-histidine, α-fluoromethyl-histidine, α-methyl-histidine, 3-pyridylalanine, 2-pyridylalanine or 4-pyridylalanine; Xaa8 is Ala, Gly, Val, Leu, Ile, Lys, Aib, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid, whereby Gly is particularly preferred; Xaa16 is Val or Leu; Xaa18 is Ser, Lys or Arg; Xaa 19 is Tyr or Gln; Xaa20 is Leu or Met; Xaa22 is Gly, Glu or Aib; Xaa23 is Gln, Glu, Lys or Arg; Xaa25 is Ala or Val; Xaa26 is Lys, Glu or Arg; Xaa27 is Glu or Leu; Xaa30 is Ala, Glu or Arg; Xaa33 is Val or Lys; Xaa34 is Lys, Glu, Asn or Arg; Xaa35 is Gly or Aib; Xaa36 is Arg, Gly or Lys or amide or absent; Xaa37 is Gly, Ala, Glu, Pro, Lys, amide or is absent, wherein these amino acids are preferably selected if the GLP-1 (fusion) peptide as defined herein is provided directly to a patient in need thereof, when treating a vascular disease or a diseases related thereto, as defined herein.

In still another specific embodiment of the invention component (I) and/or (III) of the GLP-1 (fusion) peptide as defined herein, i.e. a GLP-1 peptide or a GLP-1 fusion peptide as defined above, as encoded and secreted by cells embedded in the (spherical) core of the (spherical) microcapsules herein contains a modified GLP-1 peptide comprising the amino acid sequence of the following formula III:

Xaa7-Xaa8-Glu-Gly-Thr-Phe-Thr-Ser-Asp-Val-Ser-Xaa18-Tyr-Leu-Glu-Xaa22-Xaa23-Ala-
Ala-Xaa26-Glu-Phe-Ile-Xaa30-Trp-Leu-Val-Xaa34-Xaa35-Xaa36-Xaa37,

wherein Xaa7 is L-histidine; Xaa8 is Ala, Gly, Val, Leu, Ile, Lys; Xaa18 is Ser, Lys or Arg; Xaa22 is Gly or Glu; Xaa23 is Gln, Glu, Lys or Arg; Xaa26 is Lys, Glu or Arg; Xaa30 is Ala, Glu or Arg; Xaa34 is Lys, Glu or Arg; Xaa35 is Gly; Xaa36 is Arg or Lys, amide or is absent; Xaa37 is Gly, Ala, Glu or Lys, amide or is absent, wherein these amino acids are preferably selected if the GLP-1 (fusion) peptide as defined herein is encoded and secreted by cells embedded in the (spherical) core of the (spherical) microcapsules as used herein, to be administered to a patient in need thereof, when treating a vascular disease or diseases related thereto as defined herein, or wherein Xaa7 is L-histidine, D-histidine, desamino-histidine, 2-amino-histidine, -hydroxy-histidine, homohistidine, N-acetyl-histidine, α-fluoromethyl-histidine, α-methyl-histidine, 3-pyridylalanine, 2-pyridylalanine or 4-pyridylalanine; Xaa8 is Ala, Gly, Val, Leu, Ile, Lys, Aib, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid; Xaa 18 is Ser, Lys or Arg; Xaa22 is Gly, Glu or Aib; Xaa23 is Gln, Glu, Lys or Arg; Xaa26 is Lys, Glu or Arg; Xaa30 is Ala, Glu or Arg; Xaa34 is Lys, Glu or Arg; Xaa35 is Gly or Aib; Xaa36 is Arg or Lys, amide or is absent; Xaa37 is Gly, Ala, Glu or Lys, amide or is absent, wherein these amino acids are preferably selected if the GLP-1 (fusion) peptide as defined herein is provided directly to a patient in need thereof, when treating a vascular disease or a diseases related thereto, as defined herein.

In a particular preferred embodiment a GLP-1 (fusion) peptide, i.e. a GLP-1 peptide or a GLP-1 fusion peptide as defined above, is used, which may be encoded and secreted by cells embedded in the (spherical) core of the (spherical) microcapsule as used herein, wherein component (I) and/or (III) contain a (modified) GLP-1 peptide, which is selected from GLP-1 (7-35), GLP-1 (7-36), GLP-1 (7-36)-amide, GLP-1 (7-37) or a variant, analogue or derivative thereof. Also preferred are GLP-1 (fusion) peptides comprising in their components (I) and/or (III) a modified GLP-1 peptide having a Aib residue in position 8 or an amino acid residue in position 7 of said GLP-1 peptide, which is selected from the group consisting of D-histidine, desamino-histidine, 2-amino-histidine, hydroxy-histidine, homohistidine, N-acetyl-histidine, α-fluoromethyl-histidine, α-methyl-histidine, 3-pyridylalanine, 2-pyridylalanine and 4-pyridylalanine, preferably if the GLP-1 (fusion) peptide as defined herein is provided directly to a patient in need thereof, when treating a vascular disease or a diseases related thereto, as defined herein.

In another particular preferred embodiment a GLP-1 (fusion) peptide, i.e. a GLP-1 peptide or a GLP-1 fusion peptide as defined above, is used, which may be encoded and secreted by cells embedded in the (spherical) core of the (spherical) microcapsule as used herein, wherein both embodiments of components (I) and/or (III) of the GLP-1 (fusion) peptide as defined herein by formulae II and III may be combined with the disclosure given herein for GLP-1 (fusion) peptide. In other words, general formulae II and III may be combined e.g. with the disclosure given herein for component (II), linkers, process of manufacturing, etc.

A GLP-1 peptide or a GLP-1 fusion peptide as defined herein, preferably component (I) of the GLP-1 fusion peptide as defined herein, as well as their fragments and variants are preferably protected against proteolytic cleavage as outlined herein, more preferably against DPP-IV. Accordingly, such a GLP-1 peptide or a GLP-1 fusion peptide as defined herein as well as their fragments and variants, particularly GLP-1 fusion peptides, may contain a sequence of GLP-1, e.g. GLP-1(7-35, 36 or 37) (in case of GLP-1 fusion peptides as part of component (I) and/or (III)), resistant to the DPP-IV. In this context, resistance of a peptide to degradation by dipeptidyl aminopeptidase IV may be determined e.g. by the following degradation assay: Aliquots of the peptides are incubated at 37° C. with an aliquot of purified dipeptidyl aminopeptidase IV for 4-22 hours in an appropriate buffer at pH 7-8 (buffer not being albumin). Enzymatic reactions are terminated by the addition of trifluoroacetic acid, and the peptide degradation products are separated and quantified using HPLC or LC-MS analysis. One method for performing this analysis is: The mixtures are applied onto a Zorbax300SB-C18 (30 nm pores, 5 μm particles) 150×2.1 mm column and eluted at a flow rate of 0.5 ml/min with a linear gradient of acetonitrile in 0.1% trifluoroacetic acid (0%-100% acetonitrile over 30 min). Peptides and their degradation products may be monitored by their absorbance at 214 nm (peptide bonds) or 280 nm (aromatic amino acids), and are quantified by integration of their peak areas. The degradation pattern can be determined by using LC-MS where MS spectra of the separated peak can be determined. Percentage intact/degraded compound at a given time is used for estimation of the peptides DPP-IV stability.

In the herein context, a GLP-1 peptide or a GLP-1 fusion peptide as defined herein, preferably component (I) of a GLP-1 fusion peptide as defined herein, as well as a fragment and/or variant thereof, is defined as DPP-IV stabilized when it is 10 times more stable than the non-modified peptide sequence of GLP-1 (7-37) based on percentage intact compound at a given time. Thus, a DPP-IV stabilized GLP-1 peptide or GLP-1 fusion peptide, preferably component (I) of the GLP-1 fusion peptide as defined herein, is preferably at least 10, more preferably at least 20 times more stable than e.g. GLP-1 (7-37). Stability may be assessed by any method known to the skilled person, e.g. by adding DPP-IV to a solution of the peptide to be tested and by determining the degradation of the peptide (see herein), e.g. over a period of time, by e.g. a spectroscopic method, Western-Blot analysis, antibody screening etc.

In parallel, a GLP-1 peptide or GLP-1 fusion peptide, preferably component (I) of a GLP-1 fusion peptide as defined herein, as well as a fragment and/or variant thereof is defined as a compound, which exerts the effect of GLP-1(7-37) by e.g. binding to its native receptor (GLP-1 receptor). Preferably, a GLP-1 peptide or a GLP-1 fusion peptide, as well as a fragment and/or variant thereof as defined herein has a binding affinity to the GLP-1 receptor, which corresponds to at least 10%, preferably at least 50% of the binding affinity of the naturally occurring GLP-1 peptide. The binding affinity may be determined by any suitable method, e.g. surface plasmon resonance, etc. Moreover, it is preferred, if the GLP-1 peptide or GLP-1 fusion peptide, as well as a fragment and/or variant thereof as defined herein, evokes formation of intracellular cAMP by its binding to its extracellular receptor, which transmits the signal into the cell.

According to another preferred embodiment, the GLP-1 peptide or GLP-1 fusion peptide, preferably as defined herein, as well as the single components of the GLP-1 fusion peptide, particularly components (I), (II) and (III), and/or the entire GLP-1 fusion peptide as described herein, may be selected from modified forms of these peptides or proteins sequences. The various modified forms, particularly a modified form of the entire GLP-1 fusion peptide as described herein, may be either encoded and secreted by cells embedded in the (spherical) core of the (spherical) microcapsule as used herein or may be used directly in the treatment of a vascular disease or diseases related thereto. These modified forms are disclosed in the following and described in more detail and comprise e.g. fragments, variants, etc., of the GLP-1 peptide, preferably as defined herein or of single components of the GLP-1 fusion peptide, particularly components (I), (II) and (III), and/or the entire GLP-1 fusion peptide as described herein. In this context, fragments and/or variants of these peptides or proteins may have a sequence identity to their native peptides or proteins of at least 40%, 50%, 60%, 70%, 80%, preferably at least 90%, more preferably at least 95% and most preferably at least 99% over the whole length of the native, non-modified amino acid sequence. This likewise may be applied to the respective (coding) nucleic acid sequence.

The term “sequence identity” as defined herein typically means that the sequences are compared as follows. To determine the percent identity of two amino acid sequences, the sequences can be aligned for optimal comparison purposes (e.g., gaps can be introduced in the sequence of a first amino acid sequence). The amino acids at corresponding amino acid positions can then be compared. When a position in the first sequence is occupied by the same amino acid as the corresponding position in the second sequence, then the molecules are identical at that position. The percent identity between the two sequences is a function of the number of identical positions shared by the sequences, e.g. where a particular peptide is said to have a specific percent identity to a reference polypeptide of a defined length, the percent identity is relative to the reference peptide. Thus, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide, which is 50% identical to the reference polypeptide over its entire length. Of course, other polypeptides will meet the same criteria. Such a determination of percent identity of two sequences can be accomplished using a mathematical algorithm. A preferred, non-limiting example of a mathematical algorithm utilized for the comparison of two sequences is the algorithm of Karlin et al. (1993), PNAS USA, 90:5873-5877. Such an algorithm is incorporated into the NBLAST program, which can be used to identify sequences having the desired identity to the amino acid sequence of the invention. To obtain gapped alignments for comparison purposes, Gapped BLAST can be utilized as described in Altschul et al. (1997), Nucleic Acids Res, 25:3389-3402. When utilizing BLAST and Gapped BLAST programs, the default parameters of the respective programs (e.g., NBLAST) can be used. The sequences further may be aligned using Version 9 of the Genetic Computing Group's GAP (global alignment program), using the default (BLOSUM62) matrix (values-4 to +11) with a gap open penalty of −12 (for the first null of a gap) and a gap extension penalty of −4 (per each additional consecutive null in the gap). After alignment, percentage identity is calculated by expressing the number of matches as a percentage of the number of amino acids in the claimed sequence. The described methods of determination of the percent identity of two amino acid sequences can be applied correspondingly to nucleic acid sequences. In the context of the present invention, the term “identity” is used, however, the term “homology” may also be applied instead of the term “identity”, whereever necessary or desired.

In the context of the present invention, a “fragment” of a GLP-1 peptide, preferably as defined herein or of single components of the GLP-1 fusion peptide, particularly components (I), (II) and (III), and/or the entire GLP-1 fusion peptide as described herein, typically refers to any fragment of these peptides or proteins. Typically, such a fragment comprises a shorter peptide which retains the desired biological activity particularly of the native peptide or protein, which is, with regard to its amino acid sequence (or its encoded nucleic acid sequence), N-terminally, C-terminally and/or intrasequentially truncated compared to the amino acid sequence of the native peptide or protein (or its encoded nucleic acid sequence). Such truncation may thus occur either on the amino acid level or correspondingly on the nucleic acid level. Biologically functional fragments may be readily identified by removing amino acids (either on peptide or on amino acid level) from either end of the peptide molecule and testing the resultant peptide or protein for its biological properties as defined herein for GLP-1. Proteases for removing one or more amino acids at a time from either the N-terminal end and/or the C-terminal end of a native peptide or protein may be used to determine fragments which retain the desired biological activity. Conclusively, fragments may be due to deletions of amino acids at the peptide termini and/or of amino acids positioned within the peptide sequence.

Furthermore, a “variant” of a GLP-1 peptide, preferably as defined herein or of single components of the GLP-1 fusion peptide, particularly components (I), (II) and (III), and/or the entire GLP-1 fusion peptide as described herein, preferably comprises a protein sequence or its encoding nucleic acid sequence (or a fragment thereof), wherein amino acids of the native protein or peptide sequences are exchanged. Thereby, (a variant of) a GLP-1 peptide, preferably as defined herein, a GLP-1 fusion peptide, or of single components of the GLP-1 fusion peptide, particularly components (I), (II) and (III), and/or the entire GLP-1 fusion peptide as described herein may be generated, having an amino acid sequence which differs from the native protein or peptide sequences in one or more mutation(s), such as one or more substituted, inserted and/or deleted amino acid(s). Preferably, these variants have about the same or an improved biological activity as defined herein for GLP-1, be it a variant of GLP-1, a GLP-1 fusion peptide itself or a functional variant and/or fragment thereof, i.e. the beneficial effects known for GLP-1, e.g. its activity to powerfully reduce the damages caused by ischemia or oxygen shortage and potential death of heart tissue compared to the full-length GLP-1 peptide, GLP-1 fusion peptide or full-length single components of the GLP-1 fusion peptide, particularly components (I), (II) and (III).

Such a variant as defined herein can be prepared by mutations in the DNA sequence which encodes the synthesized variants. Any combination of deletion, insertion, and substitution may also be contained in GLP-1 peptides encoded and secreted by a cell as embedded in the (spherical) microcapsule as defined herein, provided that the finally obtained variant possesses the desired biological activity. Obviously, the mutations that will be made in the DNA encoding the variant peptide must not alter the reading frame and preferably will not create complementary regions that could produce secondary mRNA structure.

Accordingly, a variant of a GLP-1 peptide, preferably as defined herein, a GLP-1 fusion peptide, or of single components of the GLP-1 fusion peptide, particularly components (I), (II) and (III), and/or the entire GLP-1 fusion peptide as described herein, may also contain additional amino acid residues flanking the N-/or the C-terminus or even both termini of the amino acid sequence compared to the native GLP-1 peptide or native GLP-1 fusion peptide as described herein. As an example, such a variant may comprise a GLP-1 peptide or a GLP-1 fusion peptide as defined herein containing additional amino acid residues flanking the N-/or the C-terminus or even both termini of the amino acid sequence of the GLP-1 peptide or GLP-1 fusion peptide. As long as the resultant GLP-1 peptide or GLP-1 fusion peptide retains its resistance or stability towards proteases and its ability to act as defined herein, one can determine whether any such flanking residues affect the basic characteristics of the “core” peptide, e.g. by its beneficial effects known for GLP-1, by routine experimentation. The term “consisting essentially of”, when referring to a specified GLP-1 peptide as defined herein, means that additional flanking residues can be present which do not affect the basic characteristic of the specified GLP-1 peptide. This term typically does not comprehend substitutions, deletions or additions within the specified sequence.

A “variant” of a GLP-1 peptide, preferably as defined herein, a GLP-1 fusion peptide, or of single components of the GLP-1 fusion peptide, particularly components (I), (II) and (III), and/or the entire GLP-1 fusion peptide as described herein, may further refer to a molecule which comprises conservative amino acid substitutions compared to its native sequence. Substitutions in which amino acids which originate from the same class are exchanged for one another are called conservative substitutions. In particular, these are amino acids having aliphatic side chains, positively or negatively charged side chains, aromatic groups in the side chains or amino acids, the side chains of which can enter into hydrogen bridges, e.g. side chains which have a hydroxyl function. This means that e.g. an amino acid having a polar side chain is replaced by another amino acid having a likewise polar side chain, or, for example, an amino acid characterized by a hydrophobic side chain is substituted by another amino acid having a likewise hydrophobic side chain. Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra) (Urry, 1985, Absorption, Circular Dichroism and ORD of Polypeptides, in: Modern Physical Methods in Biochemistry, Neuberger et al. (ed.), Elsevier, Amsterdam).

A variant of a GLP-1 peptide, preferably as defined herein, a GLP-1 fusion peptide, or of single components of the GLP-1 fusion peptide, particularly components (I), (II) and (III), and/or the entire GLP-1 fusion peptide as described herein may thus also refer to a molecule which is substantially similar to either the entire GLP-1 peptide, preferably as defined herein, the entire GLP-1 fusion peptide, or to single components of the GLP-1 fusion peptide, particularly components (I), (II) and (III), or a fragment thereof. Such variant peptides may be conveniently prepared using methods well known in the art. Of course, such a variant would have similar beneficial effects known for the native GLP-1 peptide, preferably as defined herein, a GLP-1 fusion peptide, or of single components of the GLP-1 fusion peptide, particularly components (I), (II) and (III), and/or the entire GLP-1 fusion peptide as described herein. Such beneficial effect is, e.g. for GLP-1, its activity to powerfully reduce the damages caused by ischemia or oxygen shortage and potential death of heart tissue as the corresponding naturally-occurring GLP-1 peptide.

The types of conservative amino acid substitutions which may be contained in a variant of the GLP-1 peptide, preferably as defined herein, a GLP-1 fusion peptide, or of single components of the GLP-1 fusion peptide, particularly components (I), (II) and (III), and/or the entire GLP-1 fusion peptide as described herein, may be based on analysis of the frequencies of amino acid changes between a homologous protein/peptide of different species. Based upon such analysis, conservative substitutions may be defined herein as exchanges within one of the following five groups:
I. Small, aliphatic, non-polar or slightly polar residues: Ala, Ser, Thr, Pro, Gly;
II. Polar, negatively-charged residues and their amides: Asp, Asn, Glu, Gln;
III. Polar, positively-charged residues: H is, Arg, Lys;
IV. Large, aliphatic non-polar residues: Met, Leu, Ile, Val, Cys;
V. Large aromatic residues: Phe, Try, Trp.

Within the foregoing groups, the following substitutions are considered to be “highly conservative”: Asp/Glu; His/Arg/Lys; Phe/Tyr/Trp; Met/Leu/Ile/Val. Semi-conservative substitutions are defined to be exchanges between two of groups (I)-(IV) herein which are limited to supergroup (A), comprising (I), (II), and (III) herein, or to supergroup (B), comprising (IV) and (V) herein. Substitutions are not limited to the genetically encoded or even the naturally-occurring amino acids.

Preferred conservative amino acid substitutions of preferred groups of synonymous amino acid residues within the herein meaning particularly include, without being limited thereto:

| Amino Acid | Synonymous Residue |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, (Thr), Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, (Thr), Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, (Thr), Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

Furthermore, variants of a GLP-1 peptide, preferably as defined herein, a GLP-1 fusion peptide, or of single components of the GLP-1 fusion peptide, particularly components (I), (II) and (III), and/or the entire GLP-1 fusion peptide as described herein, may also contain amino acid substitutions, made e.g. with the intention of improving solubility (replacement of hydrophobic amino acids with hydrophilic amino acids).

In one particularly preferred embodiment a GLP-1 peptide or a GLP-1 fusion peptide as defined herein, which may be encoded and secreted by a cell embedded in the (spherical) core of the (spherical) microcapsule as defined herein, includes a GLP-1 peptide (occurring in component (I) and/or (III) of the GLP-1 fusion peptide) characterized by one or more substitution(s) at positions 7, 8, 11, 12, 16, 22, 23, 24, 25, 27, 30, 33, 34, 35, 36, or 37 of the GLP-1 peptide. As an example for the following nomenclature [Arg34-GLP-1 (7-37)] designates a GLP-1 analogue wherein its naturally occurring lysine at position 34 has been substituted with arginine.

Specifically, a GLP-1 peptide or component (I) and/or (III) of a GLP-1 fusion peptide as defined herein may correspond to variants of GLP-1(7-35, 36, 37 or 38) including, for example, Gln9-GLP-1 (7-37), Thr16-Lys18-GLP-1 (7-37), and Lys18-GLP-1 (7-37), Arg34-GLP-1 (7-37), Lys38-Arg26-GLP-1 (7-38)-OH, Lys36-Arg26-GLP-1 (7-36), Arg26,34-Lys38-GLP-1 (7-38), Arg26,34-Lys38-GLP-1(7-38), Arg26,34-Lys38-GLP-1 (7-38), Arg26,34-Lys38-GLP-1 (7-38), Arg26,34-Lys38-GLP-1 (7-38), Arg26-Lys38-GLP-1(7-38), Arg26-Lys38-GLP-1 (7-38), Arg34-Lys38-GLP-1 (7-38), Ala37-Lys38-GLP-1 (7-38), and Lys37-GLP-1 (7-37). More generally speaking, any GLP-1 variant mentioned herein (in particular according to formulae II or III) may be modified by the addition of a Lys residue at position 38.

If the GLP-1 peptide or GLP-1 fusion peptide as described herein is administered directly in the treatment of a vascular disease or diseases related thereto, the GLP-1 peptide or component (I) and/or (III) of a GLP-1 fusion peptide as defined herein may additionally correspond to variants of GLP-1(7-35, 36, 37 or 38) including Gln9-GLP-1 (7-37), D-Gln9-GLP-1(7-37), acetyl-Lys9-GLP-1 (7-37).

In a particular preferred embodiment of the invention the GLP-1 peptide or the GLP-1 fusion peptide as defined herein (with respect to component (I) or (III)) is/contains a (modified) GLP-1 peptide, which is selected from GLP-1 (7-35), GLP-1 (7-36), GLP-1 (7-36)-amide, GLP-1 (7-37) or a fragment or variant thereof.

For in vitro control purposes the GLP-1 peptide or GLP-1 fusion peptide as defined herein may be isolated from the cells (and thus from the miocrocapsules) from which it is expressed, for instance using conventional separation techniques. Thus cells may be grown under appropriate conditions, for instance including support and nutrients, in vitro, and secreted protein, i.e. the GLP-1 peptide or GLP-1 fusion peptide as defined herein, if encoded and secreted by a cell embedded in the (spherical) core of the (spherical) microcapsule or a fragment or variant thereof, is recovered from the extracellular medium. The (vector) sequences engineered for transfection into cells thus preferably include signal (peptide) sequences (see below) allowing secretion of the GLP-1 peptide or GLP-1 fusion peptide as defined herein. In this context, the GLP-1 peptide or GLP-1 fusion peptide as defined herein, if encoded and secreted by a cell embedded in the (spherical) core of the (spherical) microcapsule, or a fragment or variant thereof, may be fused to a signal sequence, either naturally endogenously or after transfection of encoding nucleic acid sequences introduced into the cell by genetic engineering methods. In an alternative, the engineered gene sequences encoding a GLP-1 peptide as defined herein do not include such signal peptide sequences, whereby the intracellularly expressed GLP-1 peptides or GLP-1 fusion peptides will typically not be secreted, and may be recovered from cells by processes involving cell lysis. In such methods the coding sequences may include purification tags allowing efficient extraction of the product peptide from the medium; tags may be cleaved off to release isolated GLP-1 peptide. However, this alternative is typically irrelevant to cells of a (spherical) microcapsule, as used according to the present invention, which are implanted into the patient and require delivery of an in vivo expressed and secreted GLP-1 peptide or GLP-1 fusion peptide as defined herein into the surrounding tissue.

Any of the herein described embodiments or features may be combined with each other, if not indicated otherwise.

The cells embedded in the (spherical) core of the (spherical) microcapsule used according to the present invention preferably encode and secrete, additionally to the GLP-1 peptide or GLP-1 fusion peptide as defined herein or its fragments or variants, the vascular endothelial growth factor (VEGF), preferably human vascular endothelial growth factor (VEGF). VEGF is a chemical signal produced by cells that stimulates the growth of new blood vessels. It is part of the system that restores the oxygen supply to tissues when blood circulation is inadequate. VEGF's normal function is to create new blood vessels during embryonic development, new blood vessels after injury, muscle following exercise, and new vessels (collateral circulation) to bypass blocked vessels. VEGF is a sub-family of growth factors, specifically the platelet-derived growth factor family of cystine-knot growth factors. They are important signaling proteins involved in both vasculogenesis (the de novo formation of the embryonic circulatory system) and angiogenesis (the growth of blood vessels from pre-existing vasculature). When VEGF is overexpressed, it can positively contribute to treatment of vascular diseases as described herein. The cells embedded in the (spherical) core of the (spherical) microcapsule used according to the present invention preferably already encode and secrete VEGF.

Accordingly, the cells embedded in the (spherical) core of the (spherical) microcapsule used according to the present invention, preferably encode and secrete the GLP-1 peptide or GLP-1 fusion peptide as defined herein, and preferably secrete VEGF, and optionally an additional factor, such as an anti-apoptotic agent, etc., as defined herein. For these purposes, the GLP-1 peptide or GLP-1 fusion peptide as defined herein or its fragments or variants as well as further additional factors, are encoded by at least one nucleic acid sequence, which is typically already contained in or transfected into the cells prior to preparation of the (spherical) core of the (spherical) microcapsule. These nucleic acid sequences thus may occur naturally in the cells or may be introduced into the cells by cell transfection techniques prior to the preparation of the (spherical) microcapsule. According to the present invention any suitable nucleic acid sequence coding for the GLP-1 peptide or GLP-1 fusion peptide as defined herein or its fragments or variants as well as further additional factors as defined herein may be used.

According to one embodiment, a nucleic acid sequence encoding the GLP-1 peptide or GLP-1 fusion peptide as defined herein, or a fragment or variant thereof, and optionally an additional factor, such as an anti-apoptotic agent, etc. as defined herein may be selected from any nucleic acid, more preferably selected from any nucleic acid suitable to encode a(t least one) peptide or protein, i.e. a coding nucleic acid, e.g. a coding DNA, selected e.g. from genomic DNA, cDNA, DNA oligonucleotides, or a coding RNA, selected e.g. from (short) RNA oligonucleotides, messenger RNA (mRNA), etc. In the context of the present invention, an mRNA is typically an RNA, which is composed of several structural elements, e.g. an optional 5′-UTR region, an upstream positioned ribosomal binding site followed by a coding region, an optional 3′-UTR region, which may be followed by a poly-A tail (and/or a poly-C-tail). An mRNA may occur as a mono-, di-, or even multicistronic RNA, i.e. an RNA which carries the coding sequences of one, two or more proteins or peptides as described herein. Such coding sequences in di-, or even multicistronic mRNA may be separated by at least one IRES sequence. The least one nucleic acid sequence may also be a ribosomal RNA (rRNA), a transfer RNA (tRNA), or a viral RNA (vRNA). Furthermore, the least one nucleic acid sequence may be a circular or linear nucleic acid, preferably a linear nucleic acid. Additionally, the at least one nucleic acid sequence may be a single- or a double-stranded nucleic acid sequence (which may also be regarded as a nucleic acid within the herein defined meaning due to non-covalent association of two single-stranded nucleic acids) or a partially double-stranded or partially single stranded nucleic acid, which are at least partially self complementary (both of these partially double-stranded or partially single stranded nucleic acids are typically formed by a longer and a shorter single-stranded nucleic acid or by two single stranded nucleic acids, which are about equal in length, wherein one single-stranded nucleic acid is in part complementary to the other single-stranded nucleic acid and both thus form a double-stranded nucleic acid in this region, i.e. a partially double-stranded or partially single stranded nucleic acid).

Due to degeneracy of the genetic code a plurality of nucleic acid sequences may code for such a a GLP-1 peptide or GLP-1 fusion peptide as defined herein, and/or for optionally an additional factor, such as an anti-apoptotic agent, etc. as defined herein. According to a preferred embodiment of the present invention a nucleic acid sequence used for transfection of cells as defined herein may comprise any nucleic acid sequence coding for the GLP-1 peptide or GLP-1 fusion peptide as defined herein and additional (functional) nucleotide sequences. In the context of the present invention, such a nucleic acid sequence is preferably suitable for transfection of a cell as defined herein. It may code for (a) the GLP-1 peptide or GLP-1 fusion peptide as defined herein, particularly for the entire GLP-1 aa sequence (GLP-1(1-37) or functional GLP-1(7-35, 36 or 37) (variant) sequences or any other GLP-1 peptide, including GLP-1 fusion peptides as defined herein, (b) optionally for a protease cleavage sequence at the N-terminus of the GLP-1 sequence according to (a) and, optionally, for a signal peptide sequence upstream from (b), (c) optionally for VEGF, if not yet secreted by the cell, and (d) optionally for a further factor as described herein. Preferably, the signal (peptide) sequence is selected from a sequence as defined below. Accordingly, the resulting amino acid sequence may be composed of a signal peptide sequence, an optional protease cleavage sequence and the GLP-1 peptide or GLP-1 fusion peptide as defined herein, or a fragment or variant thereof, and optionally an additional factor, such as an anti-apoptotic agent, etc. as defined herein, (preferably in the direction from the N- to the C-terminus). Thereby, the signal peptide sequence and the protease cleavage sequence are preferably heterologous to (the natively occurring sequences in the) host cell, and are, in case of GLP-1(5-37, 6-37, or 7-37) and variants thereof as defined herein preferably different from the amino acids 1 to 6 of native GLP-1 within the definitions of the herein proviso.

The nucleic acid sequence as defined herein may be contained in a vector. Accordingly, the cell embedded in the (spherical) core of the (spherical) microcapsule used according to the present invention may contain a vector comprising a nucleic acid as defined herein before. This vector may be used to transfect the cell as defined herein to prepare the (spherical) microcapsule as used according to the present invention. Typically, such a vector, in particular an expression vector, contains at least one nucleic acid sequence as defined herein, encoding elements (a) and optionally (b) and/or (c) and/or (d) as described herein, and, if necessary, additional elements as described herein, e.g. elements suitable for directing expression of the encoded elements (a) and optionally (b) and/or (c) and/or (d) as described herein, and optionally sequences encoding further factors, such as anti-apoptotic factors, etc. One class of vectors as used herein utilizes DNA elements that provide autonomously replicating extrachromosomal plasmids derived from animal viruses (e.g. bovine papilloma virus, polyomavirus, adenovirus, or SV40, etc.). A second class of vectors as used herein relies upon the integration of the desired gene sequences into the host cell chromosome.

Such vectors, suitable to transfect the cell prior to embedding it into the (spherical) core of the (spherical) microcapsule used according to the present invention, are typically prepared by inserting at least one nucleic acid sequence encoding elements (a) and optionally (b) and/or (c) and/or (d) as described herein, e.g. the GLP-1 peptide or GLP-1 fusion peptide as defined herein, or a fragment or variant thereof, optionally an additional factor as defined herein into suitable (empty) vectors. Such suitable (empty) vectors are known to a skilled person and may be reviewed e.g. in “Cloning Vectors” (Eds. Pouwels P. H. et al. Elsevier, Amsterdam-New York-Oxford, 1985, ISBN 0 444 904018). Suitable (empty) vectors are also intended to include any vector known to a skilled person, such as plasmids, phages, viruses such as SV40, CMV, Baculo virus, Adeno virus, Sindbis virus, transposons, IS-elements, phasmids, phagemides, cosmides, linear or circular DNA. For integration in mammalian cells linear DNA is typically used. Preferably, the vector type used for the present invention corresponds to the specific host cell requirements. Suitable commercially available expression vectors, into which the inventive nucleic acid sequences and/or vectors may be inserted, include pSPORT, pBluescriptllSK, the baculovirus expression vector pBlueBac, and the prokaryotic expression vector pcDNAII, all of which may be obtained from Invitrogen Corp., San Diego, Calif.

A vector as defined herein suitable for transfecting a cell prior to embedding it into the (spherical) core of the (spherical) microcapsule used according to the present invention, typically combines the nucleic acid sequence as defined herein with other regulatory elements, which, e.g., control expression of the encoded amino acid sequences. Such regulatory elements are e.g. 1) specific to a tissue or region of the body; 2) constitutive; 3) glucose responsive; and/or 4) inducible/regulatable. Regulatory elements herein are preferably selected from regulation sequences and origins of replication (if the vectors are replicated autonomously). Regulation sequences in the scope of the present invention are any elements known to a skilled person having an impact on expression on transcription and/or translation of the encoding nucleic acid sequences. Regulation sequences include, apart from promoter sequences so-called enhancer sequences, which may lead to an increased expression due to enhanced interaction between RNA polymerase and DNA. Further regulation sequences of inventive vectors are transcriptional regulatory and translational initiation signals, so-called “terminator sequences”, etc. or partial sequences thereof.

Generally, any naturally occurring promoter may be contained in an expression vector suitable for transfecting a cell which may be used for preparing the (spherical) microcapsule as used herein. Such promoters may be selected from any eukaryotic, prokaryotic, viral, bacterial, plant, human or animal, e.g. mammalian promoters. Suitable promoters include, for example, the cytomegalovirus promoter, the lacZ promoter, the gal 10 promoter and the AcMNPV polyhedral promoter, promoters such as cos-, tac-, trp-, tet-, trp-tet-, lpp-, lac-, lpp-lac-, laclq-, T7-, T5-, T3-, gal-, trc-, ara-, SV40-, SP6, I-PR- or the I-PL-promoter, advantageously being found in gram-negative bacteria. Additionally, promoters may be obtained from gram-positive promoters such as amy and SPO₂, yeast promoters, such as ADC1, MFa, AC, P-60, CYC1, GAPDH or mammalian promoters such as the cytomegalovirus (CMV) promoter, muscle-specific promoters including mammalian muscle creatine kinase (MCK) promoter, mammalian desmin promoter, mammalian troponin I (TNNI2) promoter, or mammalian skeletal alpha-actin (ASKA) promoter, or liver type pyruvate kinase promoters, particularly those fragments which run (−183 to +12) or (−96 to +12) (Thompson, et al. J Biol Chem, (1991). 266:8679-82.; Cuif, et al., Mol Cell Biol, (1992). 12:4852-61); the spot 14 promoter (S14, −290 to +18) (Jump, et al., J. Biol Chem, (1990). 265:3474-8); acetyl-CoA carboxylase (O'Callaghan, et al., J. Biol Chem, (2001). 276:16033-9); fatty acid synthase (−600 to +65) (Rufo, et al., J Biol Chem, (2001). 28:28); and glucose-6-phosphatase (rat and human) (Schmoll, et al., FEBS Left, (1996). 383:63-6; Argaud, et al., Diabetes, (1996). 45:1563-71), or promoters from CaM-Kinasell, Nestin, L7, BDNF, NF, MBP, NSE, beta-globin, GFAP, GAP43, tyrosine hydroxylase, Kainat-receptor-subunit 1, glutamate-receptor-subunit B, or human ubiquitin promoter B (ubiB human), human ferritin H promoter (FerH), etc. Particularly preferred promoters are of human or mammalian origin. Finally, synthetic promoters may be used advantageously. Promoter sequences, as contained in an inventive vector, may also be inducible for in vitro control purposes, to allow modulation of expression (e.g. by the presence or absence of nutrients or other inducers in the growth medium). One example is the lac operon obtained from bacteriophage lambda plac5, which can be induced by IPTG. Finally, a promoter as defined herein may be linked with a GLP-1 encoding nucleic acid sequence as defined herein, and optionally with an additional factor, such as an anti-apoptotic agent, etc. as defined herein, such that the promoter is positioned 5′ “upstream” of the GLP-1 encoding nucleic acid sequence. Preferably, human promoters are used, e.g. the human ubiquitin promoter B (ubiB human) or the human ferritin H promoter (FerH).

Enhancer sequences for upregulating expression of GLP-1 encoding nucleic acid sequences as defined herein are preferably another constituent of a vector or an expression as defined herein. Such enhancer sequences are typically located in the non-coding 3′ region of the vector. Enhancer sequences as employed in a vector as defined herein may be obtained from any eukaryotic, prokaryotic, viral, bacterial, plant, human or animal, e.g. mammalian hosts, preferably in association with the corresponding promoters as defined herein. Enhancer elements which will be most useful in the present invention are those which are glucose responsive, insulin responsive and/or liver specific. Enhancer elements may include the CMV enhancer (e.g., linked to the ubiquitin promoter (Cubi)); one or more glucose responsive elements, including the glucose responsive element (G1RE) of the liver pyruvate kinase (L-PK) promoter (−172 to −142); and modified versions with enhanced responsiveness (Cuif et al., supra; Lou, et al., J. Biol Chem, (1999). 274:28385-94); G1RE of L-PK with auxiliary L3 box (−172 to −126) (Diaz Guerra, et al., Mol Cell Biol, (1993). 13:7725-33; modified versions of G1RE with enhanced responsiveness with the auxiliary L3 box; carbohydrate responsive element (ChoRE) of S 14 (−1448 to −1422), and modifications activated at lower glucose concentrations (Shih and Towle, J Biol Chem, (1994). 269:9380-7; Shih, et al., J Biol Chem, (1995). 270:21991-7; and Kaytor, et al., J Biol Chem, (1997). 272:7525-31; ChoRE with adjacent accessory factor site of S 14 (−1467 to −1422); aldolase (+1916 to +2329) (Gregori et al., J Biol Chem, (1998). 273:25237-43; Sabourin, et al., J. Biol Chem, (1996). 271:3469-73; and fatty acid synthase (−7382 to −6970) (Rufo, et al., supra.), more preferably insulin responsive elements such as glucose-6-phosphatase insulin responsive element (−780 to −722) (Ayala et al., Diabetes, (1999). 48:1885-9; and liver specific enhancer elements, such as prothrombin (940 to −860) (Chow et al., J Biol Chem, (1991) 266: 18927-33; and alpha-1-microglobulin (−2945 to −2539) (Rouet et al., Biochem J, (1998). 334:577-84), Muscle-specific enhancers such as mammalian MCK enhancer, mammalian DES enhancer, and vertebrate troponin I IRE (TNI IRE, herein after referred to as FIRE) enhancer. Finally, a SV40 enhancer sequence may also be included.

Enhancer elements may further be used along with promoters as defined herein for upregulating expression of GLP-1 encoding nucleic acid sequences as defined herein, e.g. such promoter/enhancer combinations include e.g. the cytomegalovirus (CMV) promoter and the CMV enhancer, the CMV enhancer linked to the ubiquitin promoter (Cubi), the group of liver-specific enhancer elements comprising human serum albumin [HSA] enhancers, human prothrombin [HPrT] enhancers, alpha-1 microglobulin [A1MB] enhancers, and intronic aldolase enhancers used in combination with their corresponding promoters, or HSA enhancers used in combination with a promoter selected from the group of a CMV promoter or an HSA promoter, enhancer elements selected from the group consisting of human prothrombin [HPrT] and alpha-1 microglobulin [A1MB] used in combination with the CMV promoter enhancer elements selected from the group consisting of human prothrombin [HPrT] and alpha-1 microglobulin [A1MB] used in combination with the alpha-1-anti trypsin promoter, etc.

Furthermore, a vector as defined herein suitable for transfecting a cell which may be used as constituent of the (spherical) microcapsule as used according to the present invention, may contain transcriptional and/or translational signals, preferably transcriptional and/or translational signals recognized by an appropriate host, such as transcriptional regulatory and translational initiation signals. Transcriptional and/or translational signals may be obtained from any eukaryotic, prokaryotic, viral, bacterial, plant, preferably human or animal, e.g. mammalian hosts, preferably in association with the corresponding promoters as defined herein. A wide variety of transcriptional and translational regulatory sequences may be employed therefore, depending upon the nature of the host to the extent that the host cells recognizes the transcriptional regulatory and translational initiation signals associated with a GLP-1 encoding nucleic acid sequence, and optionally an additional factor as defined herein. The 5′ region adjacent to the naturally occurring GLP-1 encoding nucleic acid sequence may be retained and employed for transcriptional and translational regulation in an inventive vector. This region typically will include those sequences involved with initiation of transcription and translation, such as the TATA box, capping sequence, CAAT sequence, and the like. Typically, this region will be at least about 150 base pairs long, more typically about 200 bp, and rarely exceeding about 1 to 2 kb.

Transcriptional initiation regulatory signals suitable for a vector as defined herein may be selected that allow to control repression or activation such that expression of the GLP-1 encoding or nucleic acid sequences as defined herein, and optionally of an additional factor as defined herein, can be modulated. One such controllable modulation technique is the use of regulatory signals that are temperature-sensitive in order to repress or initiate expression by changing the temperature. Another controllable modulation technique is the use of regulatory signals that are sensitive to certain chemicals. These methods are preferably to be used in in vitro procedures, e.g. when preparing the necessary constructs. Furthermore, transcriptional initiation regulatory signals may be use herein, which allow control repression or activation of expression in vivo without any further means from outside the cell, e.g. to obtain a transient expression in the encapsulated cells. Such transcription and/or translational signals include e.g. transcriptional termination regulatory sequences, such as a stop signal and a polyadenylated region. Furthermore, transcriptional termination regulatory sequences may be located in the non-coding 3′ region of a vector as defined herein containing the GLP-1 encoding nucleic acid sequence. Suitable termination sequences can include, for example, the bovine growth hormone, SV40, lacZ, EF1 alpha and AcMNPV polyhedral polyadenylation signals.

The expression vectors suitable for transfecting a cell which may be used for preparing the (spherical) microcapsule as used according to the present invention, may also include other sequences for optimal expression of the GLP-1 encoding or nucleic acid sequences as defined herein, and optionally of an additional factor as defined herein. Such sequences include those encoding signal (peptide) sequences, i.e. which encode N-terminally located peptide sequences that provide for passage of the secreted protein into or through a membrane; which provide for stability of the expression product; and restriction enzyme recognition sequences, which provide sites for cleavage by restriction endonucleases. All of these materials are known in the art and are commercially available (see, for example, Okayama (1983), Mol. Cell. Biol., 3: 280).

As defined herein “a signal sequence” is a signal (peptide) sequence which typically comprises about 8 to 30 amino acids, or 15 to 30 mino acids, located—within the definitions of the herein proviso regarding amino acids 1 to 6 of GLP-1—at the N-terminus of the expressed GLP-1 (fusion) peptide and enables the GLP-1 peptide to be secreted, i.e. to pass through a cell membrane. Such a signal (peptide) sequence may include the signal sequence normally associated with the wild type GLP-1 precursor protein (i.e., the signal sequence(s) of the full length proglucagon precursor molecule), as well as signal (peptide) sequences which are not normally associated thereto, i.e. which are heterologous to the wild type GLP-1 precursor protein (i.e., the signal (peptide) sequence(s) of the full length proglucagon precursor molecule). A “signal (peptide) sequence” as defined herein can be, for example, a signal peptide sequence or a leader sequence (e.g. a secretory signal (and leader) sequence). Furthermore, signal (peptide) sequences as defined herein preferably provide for cleavage of the (GLP-1) precursor peptide by a protease, e.g. a signal (peptide) sequence protease. Upon cleavage of the signal (peptide) sequence from the (GLP-1) precursor peptide by the protease a biologically active GLP-1 peptide as defined herein is produced. Such a signal (peptide) sequence generally comprises a region which encodes a cleavage site recognized by a protease for cleavage. Alternatively, a region which encodes a cleavage site recognized by a protease for cleavage can be introduced into the signal (peptide) sequence. Furthermore, additional (one or more) sequences which encodes a cleavage site recognized by a protease for cleavage can be added to the signal (peptide) sequence.

Examples of signal (peptide) sequences which can be encoded by a vector as defined herein include a signal (peptide) sequence derived from a secreted protein such as GLP-1 or other than GLP-1, VEGF, or from a cytokine, a clotting factor, an immunoglobulin, a secretory enzyme or a hormone (including the pituitary adenylate cyclase activating polypeptide (PACAP)/glucagon superfamily) and a serum protein. For example, a signal (peptide) sequence as defined herein can be derived from secreted matrix metalloproteinases (MMP), e.g. a stromelysin leader sequence, from secreted human alkaline phosphatase (SEAP), pro-exendin, e.g. a proexendin-4 leader sequence, pro-helodermin, pro-glucose-dependent insulinotropic polypeptide (GIP), pro-insulin-like growth factor (IGF1), preproglucagon, alpha-1 antitrypsin, insulin-like growth factor 1, human factor IX, human lymphotoxin A (Genbank Accession no. BAA00064), or human clusterin (Genbank Accession No. AAP88927). Particular examples of signal (peptide) sequences as defined herein are sequences which include a coding region for a signal for precursor cleavage by signal peptidase, furin or other prohormone convertases (e.g., PC3). For example, a signal (peptide) sequence which is cleaved by furin (also known as PACE, see U.S. Pat. No. 5,460,950), other subtilisins (including PC2, PC1/PC3, PACE4, PC4, PC5/PC6, LPC/PC7IPC8/SPC7 and SKI-1; Nakayama, Biochem. J., 327:625-635 (1997)); enterokinase (see U.S. Pat. No. 5,270,181) or chymotrypsin can be introduced into the signal (peptide) sequence as defined herein. The disclosure of each of these documents is hereby incorporated herein by reference. Furin is a ubiquitously expressed protease that resides in the trans-golgi and processes protein precursors before their secretion. Furin cleaves at the COOH-terminus of its consensus recognition sequence, Arg-X-Lys-Arg or Arg-X-Arg-Arg, (Lys/Arg)-Arg-X-(Lys/Arg)-Arg and Arg-X-X-Arg, such as an Arg-Gln-Lys-Arg. These amino acid sequences are a signal for precursor cleavage by the protease furin. Thus, a heterologous signal (peptide) sequence can also be synthetically derived from a consensus sequence compiled from signal (peptide) sequences (e.g., a consensus sequence compiled from secreted proteins that are cleaved by signal peptidase).

Additionally to regulation sequences as defined herein, an autonomously replicating vector as defined herein typically comprises an origin of replication. Suitable origins of replication include, without being limited thereto, e.g. ColE1, pSC101, SV40, pMPI (ori pMPI) and M13 origins of replication, etc.

Preferably, a vector as defined herein, suitable for expression of the GLP-1 encoding nucleic acid sequences of the cells of the (spherical) microcapsules as defined herein, and optionally of an additional factor as defined herein, may additionally contain a suicide gene. In the context of the present invention “a suicide gene” is preferably capable to stop the therapy with (spherical) microcapsules, as used herein, by killing the suicide gene harbouring cell contained in the (spherical) core of the (spherical) microcapsule upon administering a specific substance. In other words, a suicide gene suitable for the present invention may be activated by administering an exogenous activator that typically does not occur in the human or animal body. In this case, typically the suicide gene initiates a cascade causing the cell to undergo an apoptotic event. Alternatively, a suicide gene suitable for the present invention may metabolize an administered exogenous non-toxic prodrug that typically does not occur in the human or animal body. Metabolism of the exogenous non-toxic prodrug preferably renders the prodrug to a cell toxin. The suicide gene may be contained on the same vector encoding the GLP-1 peptide of GLP-1 fusion peptide as defined herein or alternatively on a second vector. Furthermore, the suicide gene may be regulated by control and regulatory elements of any kind, e.g. control and regulatory elements such as promoters, enhancers, etc. as mentioned herein as constituents of expression vectors, or by their naturally occurring control and regulatory elements. Preferably, suicide genes are selected according to the present invention, which allow any of the herein control mechanisms, e.g. suicide genes selected from cytosin deaminase (CD), uracil phosphoribosyl transferase (UPRTase), HSV thymidine kinase (HSV-Tk), suicide genes which may be induced by addition of tetracycline such as the bacterial Tet repressor protein (TetR), etc. As a particular example the cytosine desaminase (CD) may be used. The cytosine desaminase (CD) typically occurs in a variety of organisms and is capable of transforming 5-fluorocytosin (5-FC) into 5-fluorouracil (5-FU), which represents a common chemotherapeutic agent. 5-Fluorouracil (5-FU) is highly toxic for the organism whereas its prodrug 5-fluorocytosin (5-FC) is not toxic to cells. 5-Fluorouracil (5-FU) is subsequently phosphorylated by cellular kinases and is capable of abrogating the cells RNA synthesis. Thus, the prodrug 5-fluorocytosin (5-FC) represents an excellent tool for inducing suicide of a specific cell. Furthermore, 5-Fluoro-dUMP acts as antifolate agent and inhibits the enzyme thymidylat synthase, which catalyses methylation of dUMP to dTMP in the de novo synthesis path of desoxyribonucleotides. Thereby, inhibition of DNA synthesis in the cell may be achieved. Also preferably, the HSV-1 thymidin kinase (ATP: Thymidin-5-phosphotransferase) and its corresponding prodrug ganciclovir (GCV) may be used. The guanosin analog GCV is specifically phosphorylated and inhibits elongation of DNA synthesis and thus leads to suicide of the cell.

Transfection of the vectors or nucleic acids as defined herein, encoding a GLP-1 peptide or GLP-1 fusion peptide and optionally an additional factor, into suitable cells used for preparation of (spherical) microcapsules as defined herein, may be accomplished by any method known to a skilled person (see e.g. Maniatis et al. (2001) Molecular Cloning: A laboratory manual, Cold Spring Harbor Laboratory Press, Cold Spring Harbor, N.Y.). If vectors are transfected into suitable cells as defined herein, the vector is preferably present in the form of a plasmid DNA, which carries a GLP-1 or GLP-1 fusion peptide encoding nucleic acid. The plasmid DNA is preferably a circular plasmid DNA. Suitable transfection methods include, without being limited thereto, e.g. electroporation techniques including modified electroporation techniques (e.g. nucleofection), calcium phosphate techniques, e.g. the calcium phosphate co-precipitation method, the DEAE-Dextran method, the lipofection method, e.g. the transferring-mediated lipofection method, etc. Preferably, transfection is carried out with plasmid DNA carrying a vector as defined herein using a modified electroporation technique (e.g. nucleofection).

The vector as defined herein or, alternatively, the nucleic acid, encoding a GLP-1 peptide or GLP-1 fusion peptide, or a fragment or variant thereof as defined herein, and optionally an additional factor as defined herein, may furthermore be complexed, e.g. for transfection with at least one synthetic polymer or a natural polymer, e.g. polyamino acids, or may be conjugated thereto. At least one polymer constituent may be covalently coupled to the vector as defined herein or, alternatively, the nucleic acid encoding a GLP-1 peptide or GLP-1 fusion peptide, or a fragment or variant thereof as defined herein, and optionally an additional factor as defined herein. “Conjugated” in the meaning of the present invention is intended to mean “chemically coupled”. “Chemically coupled” is intended to mean coupled via covalent or non-covalent bonding. While covalent bonding may also be utilized, non-covalent bonding is preferred for transfection purposes. Thereby, the polymer constituent may be linked to the fusion peptide via complexation without covalent linkage, e.g. via hydrogen bonding or electrostatic, hydrophobic, etc., interaction.

The polymer used herein for coupling the vector as defined herein or, alternatively, the nucleic acid, encoding a GLP-1 peptide or GLP-1 fusion peptide, or a fragment or variant thereof as defined herein, and optionally an additional factor as defined herein, may be a physiologically acceptable polymer which includes polymers which are soluble in an aqueous solution or suspension and have no negative impact, such as side effects, to mammals upon administration of the fusion peptide in a pharmaceutically effective amount. There is no particular limitation to the physiologically acceptable polymer used according to the present invention. The polymer may be of synthetic nature or may be a naturally occurring polymer (e.g. a protein).

More generally, the synthetic polymer used with a vector as defined herein or, alternatively, the nucleic acid encoding a GLP-1 peptide or GLP-1 fusion peptide, or a fragment or variant thereof as defined herein, and optionally an additional factor as defined herein, is preferably selected from alkylene glycols, such as polyethylene glycol (PEG), polypropylene glycol (PPG), copolymers of ethylene glycol and propylene glycol, polyoxyethylated polyol, polyolefinic alcohol, polyvinylpyrrolidone, polyhydroxyalkyl methacrylamide, polyhydroxyalkyl methacrylate, such as polyhydroxyethylene methycrylate, polyacrylate, polysaccharides, poly([alpha]-hydroxy acid), polyvinyl alcohol, polyphosphazene, polyoxazoline, poly(N-acryloylmorpholine), polyvinylethyl ether, polyvinlyacetal, polylactic glycolic acid, polylactic acid, lipid polymer, chitin, hyaluronuic acid, polyurethyne, polysialic acid, cellulose triacetate, cellulose nitrate and combinations of any of the foregoing.

The present invention also provides a method for preparing the (spherical) microcapsules as used according to the present invention. These (spherical) microcapsules are preferably prepared according to two or more method steps. According to a method step 1) a core is prepared as disclosed above. According to a method step 2) the core as prepared according to method step 1) is coated by one or more surface coating layer(s). Further optional steps may comprise repetition of method step 2) for the preparation of additional surface coating layers. Preferably, a step identical to method step 2) is carried out for each of such additional surface coating layers. Further optional steps may include washing steps subsequent to preparation of the spherical microcapsule.

Typically, a core as disclosed herein is prepared according to method step 1) for preparing (spherical) microcapsules, as used according to the present invention. Such a core is composed of cross-linked polymer and GLP-1 encoding and secreting cells as defined above, which have been transfected according to a method as disclosed herein. According to method step 1), a mixture (suspension) of the soluble form of the polymer, e.g. the soluble form of an alginate (e.g. potassium or sodium alginate in physiological saline solution), and of GLP-1 encoding and secreting cells is typically prepared, preferably in a concentration as defined herein for the (spherical) core, e.g. of 1×10⁵ up to 6×10⁷ cells, per ml polymer solution.

As a typical technique the homogenic cell/polymer suspension (e.g. cell/alginate suspension) may be pressed via an air injected spray nozzle, consisting of three channels, which are arranged concentrically as three concentric rings around a common centre: an inner channel, an intermediate channel and an outer channel (air ring). Preferably hollow needles are used for the inner channel having an inner diameter of 50 μm up to 2,000 μm. The intermediate channel typically has an inner diameter of 60 μm to 4,000 μm, and the outer channel (air ring) preferably has an inner diameter of 100 μm to 5,000 μm. Exclusively the inner channel and the outer channel (air ring) are used in method step 1) for preparing the core of the (spherical) microcapsule, as used according to the present invention. Thus, a spray nozzle merely consisting of two channels (an inner and an outer channel) may be used in method step 1) as well. Typically, no material flows through the intermediate channel, if an air injected spray nozzle with three channels is used. The suspension of the cell/polymer solution is typically pressed with a speed of 10 μl/min to 5 ml/min through the inner channel leading to droplets at the outlet of the channel, which tear off due to the air flow provided by the outer channel (air ring), having a speed of typically 0.5 l/min to 10 l/min. Droplets containing cells and non-cross-linked polymer solution fall down into a cross-linker containing solution (precipitation bath), which is typically positioned in a distance of about 4 cm to about 60 cm under the outlet of the air injected spray nozzle. The droplet preferably rounds during dropping down, thereby receiving a substantially spherical geometrical form. The cross-linker effects ionical cross-linking of the polymers and the core of the spherical (water insoluble) microcapsule is initially formed having a diameter as defined herein for the (spherical) core. The diameter of the core of the (spherical) microcapsule is dependent on size and geometry of the chosen channels used in method step 1). The cross-linker containing solution (precipitation bath) is preferably composed of bivalent cations, e.g. calcium or barium ions (5-100 mM) or other bivalent or multivalent cations, if alginates are used as polymers. Furthermore, the precipitation bath preferably contains a buffer substance (e.g. 1 mM-10 mM histidine) and sodium chloride (e.g. 290 mOsmol±50 mOsmol). Other suitable cross-linkers and buffers known in the art may be used herein, if other polymers than alginates are used.

Method step 1) provides the core of the (spherical) microcapsule composed of cross-linked polymers and cells as defined herein. Subsequent to method step 1) optional method step(s) may include a washing step. The core of the (spherical) microcapsule, as used according to the present invention, is e.g. washed with a physiological saline solution or any other suitable washing solution and, if applicable, the core is incubated in a sodium sulfate solution, preferably in a sodium sulfate solution according to U.S. Pat. No. 6,592,886, the disclosure of which is incorporated herein by reference. Separation of the cores of the (spherical) microcapsules, as used according to the present invention, from the precipitation bath and/or the washing bath is typically is carried out using a centrifuge or any other suitable method.

According to method step 2) the core of the (spherical) microcapsule, as used according to the present invention, prepared by method step 1) is coated with a surface coating layer substantially of cross-linked polymer. Accordingly, the core of the (spherical) microcapsule, prepared by step 1), is added to a polymer solution containing non-crosslinked polymers as disclosed herein comprising no cells. Preferably, the polymers are provided in their non-cross-linked form in a concentration as defined herein. Typically, this mixture containing the polymer solution and the core of the (spherical) microcapsule is pressed through the inner channel of the herein-described air injected spray nozzle, e.g. with a speed of 15 μl/min to 2 ml/min, preferably 10 μl/min to 5 ml/min. Simultaneously, a pure non-cross-linked polymer solution without cells, preferably a solution comprising about 0.1% to about 4% (w/v) polymer, e.g. an alginate solution without any cells, is pressed through the intermediate channel with a speed of typically 15 μl/min to 2 ml/min, preferably 10 μl/min to 5 ml/min. Thereby, droplets are formed at the end of the intermediate channel, containing the core and a surface of non-polymerized polymer. These droplets tear off due to the air flow provided via the outer channel (air ring) having a speed of typically 0.5 l/min to 10 l/min. The polymer concentration of the core of the (spherical) microcapsule, the polymer solution, into which the core of the (spherical) microcapsules is added, and the polymer concentration of the surface coating may differ (see herein). The droplets containing the core of the (spherical) microcapsules (prepared according to method step 2) fall into a solution containing the cross-linker (precipitation bath) as defined herein. During dropping down, the droplet preferably rounds to an approximately spherical geometrical form. The cross-linker affects an ionic cross-linkage of the polymers analogous to method step 1). Thereby, water insoluble (spherical) microcapsules are formed having a diameter as defined herein, preferably of total diameter (particle size) of the (spherical) microcapsule of about 100 μm to about 200 μm, more preferably a total diameter of about 115 μm to about 185 μm, even more preferably a total diameter of about 130 μm to about 170 μm, and most preferably a total diameter of about 145 μm to about 155 μm, e.g. about 150 μm. The total diameter of (spherical) microcapsules obtainable by method step 2) is dependent from size and geometry of the chosen channels, as used herein. In order to prepare (spherical) microcapsules as defined herein, with more than one surface coating layer, i.e. the (spherical) microcapsules containing the core as defined herein and 2, 3, 4, 5, 5-10 or more surface coating layers, method step 2) may be repeated as often as necessary. Those further surface coating layers are defined within the herein diameter ranges.

Subsequent to method step 2) one or more optional washing steps may follow as defined herein.

According to a further aspect the present invention also provides a method of treatment of a vascular disease in an animal, preferably a mammal. Such a method of treatment may therefore be used in the field of either human medicine or veterinary medicine. In the context of the present invention the term mammal typically comprises any animal and human, preferably selected from the group comprising, without being restricted thereto, humans and (mammalian) (non-human) animals, including e.g. pig, goat, cattle, swine, dog, cat, donkey, monkey, ape or rodents, including mouse, hamster and rabbit, cow, rabbit, sheep, lion, jaguar, leopard, rat, pig, buffalo, dog, loris, hamster, guinea pig, fallow deer, horse, cat, mouse, ocelot, serval, etc. Such a treatment typically occurs by administration of (spherical) microcapsules as defined herein to a patient in need thereof, particularly by the administration of cells as defined herein, e.g. mesenchymal stem cells or mesenchymal stromal cells, or any other cell (type), that may be used in the context of the present invention, encoding and secreting a GLP-1 peptide as defined herein, a GLP-1 fusion peptide as defined herein, or a fragment or variant thereof, wherein these cells are encapsulated in a (spherical) microcapsule as defined herein to prevent a response of the immune system of the patient to be treated. Preferably, the (spherical) microcapsule as well as all its components as used in the inventive method, e.g., polymers of the polymer matrix of the core or the surface coating, etc., is as defined above.

Treatment of vascular diseases in the context of the present invention preferably comprises prevention, treatment, and/or amelioration of vascular diseases or of conditions associated therewith. Non-limiting examples of such vascular diseases or conditions include vascular diseases, preferably peripheral vascular diseases (PVD), also known as peripheral artery diseases (PAD) or peripheral artery occlusive diseases (PAOD), includes all diseases caused by the obstruction of large arteries in the arms and legs; as well as a subset of diseases classified as microvascular diseases resulting from episodal narrowing of the arteries (raynauds), or widening thereof (erythromelalgia), such as vascular spasms. Non-limiting examples of such vascular diseases or conditions, preferably peripheral vascular diseases (PVD), also include vein graft diseases, which also can be defined as a peripheral vascular disease. Such vein graft diseases typically include diseases involving the progressive degradation and build up atheroma (thickening of the arteries from the depositing of plaque on the artery walls.) and clots within the ever thickening wall of veins which are used as arteries during surgical bypass operations. Often, over days to less than a decade, the sections of veins which are used as bypass graphs (sewn into the side of arteries as another path for blood to flow through) deform, narrow and occlude. Non-limiting examples of such vascular diseases or conditions, preferably peripheral vascular diseases (PVD), furthermore include venous or vein disorders or diseases, preferably vein disorders involving veins of the circulatory system, such as varicose and spider veins, which typically occur when returning to the heart pools inside a vein causing congestion and enlargement of the vein, deep vein thrombosis, thrombophlebitis, vein thromboembolism (DVT), which typically occurs due to blood clots in veins and may lead to loose and travel to the lungs, also called pulmonary embolisms (PE), sometimes with a fatal outcome, chronic venous insufficiency or venous stasis ulcers, preferably caused by a venous reflux and/or a damage of the vein valves, e.g. due to vein blockages caused by clots, and further venous diseases, e.g. veneous diseases due to advanced clotting problems, severe chronic venous insufficiency, venous stasis ulcers, venous thoracic outlet syndrome, congenital venous malformation, veneous diseases caused by insufficient vascularization, etc.

Preferably, vascular diseases, preferably peripheral vascular diseases (PVD), as defined above do not include cardiovascular diseases or diseases caused by stroke, (acute) myocardial infarct, heart failure, cardiomyopathy and/or coronary diseases, which are preferably excluded from the scope of the present invention by way of disclaimer.

According to a further aspect the present invention therefore provides the use of cells according to the invention as described herein wherein vascular diseases, preferably peripheral vascular diseases (PVD), do not include cardiovascular diseases or diseases caused by stroke, (acute) myocardial infarct, heart failure, cardiomyopathy and/or coronary diseases.

Nevertheless, this does not affect the use of the cells according to the invention for treatment of vascular diseases as a medication for preventing such cardiovascular diseases or diseases caused by stroke, (acute) myocardial infarct, heart failure, cardiomyopathy and/or coronary diseases occurring as after-effects of other vascular diseases.

According to a further aspect the present invention thus provides the use of cells, encoding and secreting at least GLP-1, a fragment or variant thereof, and additionally secreting VEGF for the preparation of a medicament for the treatment of vascular diseases according to the invention, wherein the vascular disease is peripheral vascular disease, aneurysm, renal artery disease, Raynaud's phenomenon, Buerger's disease, peripheral venous disease, varicose veins, venous blood clots, deep vein thrombosis, pulmonary embolism, chronic venous insufficiency, vein graft disease or lymphedema, preferably peripheral vascular disease or vein graft disease.

Thereby it is particularly advantageous not only to provide a medicament for healing said vascular diseases but also to prevent other diseases occurring as after-effects, such as cardiovascular diseases or diseases caused by stroke, (acute) myocardial infarct, heart failure, cardiomyopathy and/or coronary diseases or leading to stroke, (acute) myocardial infarct, heart failure, cardiomayopathy and/or coronary diseases, or stroke, myocardial infarct, heart failure, cardiomyopathy and/or coronary diseases as such.

A method for prevention, treatment, and/or amelioration of a vascular disease or of conditions associated therewith as defined herein typically comprises administering the cells, encapsulated in a (spherical) microcapsule as defined herein or the (spherical) microcapsule as defined herein, or administering the pharmaceutical composition containing such (spherical) microcapsule, to a patient in need thereof. A patient in need thereof is typically, e.g., an animal, preferably a mammal, such as a human being. Administration in the context of the herein method of treatment typically occurs in a “safe and effective” amount of the active agent, i.e. the cells, encapsulated in a (spherical) microcapsule as defined herein, or the (spherical) microcapsule as defined herein. As used herein, “safe and effective amount” means an amount of these cells, encapsulated in a (spherical) microcapsule as defined herein, or the (spherical) microcapsule as defined herein, that is sufficient to significantly induce a positive modification of a disease or disorder as mentioned herein. At the same time, however, a “safe and effective amount” is small enough to avoid serious side-effects, that is to say to permit a sensible relationship between advantage and risk. The determination of these limits typically lies within the scope of sensible medical judgment. In the context of the present invention the expression “safe and effective amount” preferably means an amount of the cells, encapsulated in a (spherical) microcapsule as defined herein, or the (spherical) microcapsule as defined herein that is suitable to exert beneficial effects known for GLP-1, e.g. its activity to powerfully reduce the damages caused by ischemia or oxygen shortage and potential death of heart tissue without the need of repeated administration of GLP-1 peptide(s) and/or the risk of an undesired immune response against e.g. implanted GLP-1 expressing allogenic cells. A “safe and effective amount” of the cells, encapsulated in a (spherical) microcapsule as defined herein, or the (spherical) microcapsule as defined herein, will furthermore vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the administering doctor.

Typically, (spherical) microcapsules as contained in the inventive pharmaceutical composition secrete about 0.2 μg GLP-1 peptide as defined herein per day per ml of (spherical) microcapsules. Thus, a dosage range may be e.g. in the range from about 0.01 μg to 20 mg of secreted biologically active GLP-1 peptide per day (even though higher amounts in the range of 1-100 mg are also contemplated), such as in the range from about 0.01 μg to 10 mg per day, preferably in the range from 0.01 μg to 5 mg per day and even more preferably in the range from about 0.01 μg to 1 mg per day and most preferably in the range from about 0.01 μg to 500 μg per day.

Administration in the context of the herein described method of treatment typically occurs by providing the cells, encapsulated in a (spherical) microcapsule as defined herein, or the (spherical) microcapsule as defined herein, or the pharmaceutical composition containing such (spherical) microcapsule, to or into a specific administration site of the patient to be treated. Such a specific administration site is typically a vascular vessel, e.g. a blood vessel, an artery, a vein, preferably selected from blood vessels throughout the body, blood vessels of or around the heart, e.g. arterioles feeding the heart muscle or tissue, biocompatible stents implanted into the heart, vein grafts, arterioles feeding the myocardium or myocardial tissue, the LAD=left anterior descending (LAD) coronary artery), or other coronary arteries, or a connective tissue related to such a vascular vessel, blood vessel, artery, or vein, etc. e.g. the adventitia, peri-adventitia, tunica adventitia or tunica externa (the outermost connective tissue of the vascular vessel) of such a vascular vessel, blood vessel, artery, or vein, etc.

If administration is carried out, e.g. by administering the (spherical) microcapsule onto or into a blood vessel, or in the vicinity of vessels intra-muscularly or subcutaneously, the inventive (spherical) microcapsule are typically administered in an amount and a time, which prevents or ameliorates occlusion of the vascular vessel and any embolic effect, such as a microinfarcts, etc. This may be achieved by e.g. administering the total amount of (spherical) microcapsules to be administered, e.g. about 5,000 to about 1,000,000 beads, about 10,000 to about 750,000 beads, about 10,000 to about 500,000 beads, about 10,000 to about 250,000 beads, or about 10,000 to about 100,000 beads, e.g. about 40,000 to about 100,000 beads, e.g. about 40,000, about 50,000, about 60,000, about 70,000, about 80,000, about 90,000 or about 100,000 beads, about 60,000 beads e.g. corresponding to e.g. about 3 to 4 million cells, or about 100,000 to about 300,000 beads, e.g. about 100,000, about 150,000, about 200,000, about 250,000 or about 300,000 beads, or any range formed by any two of these values. Administration preferably occurs in a slow speed or a time staggered mode. As an example, up to 10,000,000 beads may be slowly administered into the left anterior descending (LAD) coronary artery without causing an infarct.

Accordingly, administration of the (spherical) microcapsule as defined herein or the pharmaceutical composition containing such a (spherical) microcapsule into a specific administration site as defined herein may be carried out using different modes of administration. The (spherical) microcapsules as defined herein or the inventive pharmaceutical composition containing such a (spherical) microcapsule can be administered, for example, systemically or locally. Routes for systemic administration in general include, for example, transdermal or parenteral routes, including intravenous, subcutaneous, and/or intraarterial injections. Routes for local administration in general include, e.g., topical administration routes but also transdermal, intravascular, adventitial, periadventitial, intramuscular and/or subcutaneous injections. More preferably, the cells, encapsulated in a (spherical) microcapsule as defined herein, or the (spherical) microcapsule as defined herein, or the pharmaceutical composition containing such (spherical) microcapsule, may be administered by an intravascular, an adventitial, a peri-adventitial, an intravenous, and/or an intraarterial injection.

Other modes of administration, which may be suitable for treatment of any of the herein mentioned diseases or disorders, include transplantation of the (spherical) microcapsules as defined herein or of an inventive pharmaceutical composition preferably into an administration site as defined above. In this context, the (spherical) microcapsules or the inventive pharmaceutical composition as defined herein, may be directly delivered to the affected site of the heart (an administration site as defined herein) by interventional means, e.g. using a catheter to navigate to the affected area and implant the (spherical) microcapsules as defined herein or the inventive pharmaceutical composition by injection into the administration site. Implantation could be performed during routine methods, preferably via micro-invasive or non-invasive methods. Implantation may also occur by intravascular delivery through veins or arterioles feeding the affected area.

Without being limited thereto, the (spherical) microcapsules as defined herein or the inventive pharmaceutical composition may be administered e.g. via injection by applying an appropriate injection needle such as injection needles having a size of from 12 to 26 G, more preferably of from 18 to 22 G or e.g. by transplanting the cells or the (spherical) microcapsules as defined herein, preferably formulated in a suitable form, using surgical devices, such as scalpels, injection needles as defined herein, etc. According to a particular example, which shall not be regarded as limiting to the present embodiment, a patient in need thereof, suffering from a vascular disease or any disease associated thereto or as disclosed herein may receive an injection or implantation of the cells or the (spherical) microcapsules as defined herein into a site of administration as defined herein, etc.

Treating or preventing a vascular disease and disorders related thereto as defined herein using (spherical) microcapsules or an inventive pharmaceutical composition as defined herein preferably results from the beneficial effects of GLP-1 and preferably VEGF, e.g. the angiogenic activity of GLP-1 or the vascular growth effects of VEGF.

According to the knowledge of the present inventors, without being bound thereto, the in situ beneficial effects of (spherical) microcapsules encoding and secreting GLP-1 is at least in part based on the fact that GLP-1 stimulates proliferation of endothelial cells through PKA-PI3K/Akt-eNOS activation pathways via a GLP-1 receptor-dependent mechanism, working synergistically with other angiogenic factors such as VEGF to enhance new blood vessel formation in a locoregional manner around the vicinity of the beads, as demonstrated in the examples evidenced herein.

The invention furthermore encompasses use of cells as defined herein or of (spherical) microcapsules as defined herein for the manufacture of a product, e.g. a pharmaceutical composition or a kit, for the treatment of a vascular disease in an animal, preferably a mammal, such as a human being. The cells as used in such a treatment may be cells as defined herein, e.g. mesenchymal stem cells or mesenchymal stromal cells, or any further cell, that may be used in the context of the present invention, encoding and secreting at least GLP-1, a fragment or variant thereof, and preferably secreting additionally VEGF, wherein these cells, are encapsulated in a (spherical) microcapsule to prevent a response of the immune system of the patient to be treated.

Another aspect of the present invention is a pharmaceutical composition containing cells as defined herein, encoding and secreting at least GLP-1, a fragment or variant thereof, and preferably secreting additionally VEGF, wherein these cells are encapsulated in a (spherical) microcapsule as defined herein, or a pharmaceutical composition containing (spherical) microcapsules as defined herein. Such a pharmaceutical composition may be applied to a patient suffering from the herein defined disorders preferably to the administration sites as defined herein according to an administration mode as defined herein.

Preparation of a pharmaceutical composition which contains cells as defined herein, encoding and secreting at least GLP-1, a fragment or variant thereof, and preferably secreting additionally VEGF, wherein these cells, are encapsulated in a (spherical) microcapsule as defined herein, or a pharmaceutical composition containing (spherical) microcapsules as defined herein as an “active ingredient”, is generally well understood in the art, as e.g. exemplified by U.S. Pat. Nos. 4,608,251; 4,601,903; 4,599,231; 4,599,230; 4,596,792; and 4,578,770, all incorporated herein by reference.

Typically, pharmaceutical compositions are prepared as injectables either as liquid solutions or suspensions, preferably containing water (aqueous formulation) or may be emulsified. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

For intramuscular, intravenous, cutaneous or subcutaneous injection, or any further injection at the site of affliction as defined herein, the cells or (spherical) microcapsules as defined herein will be in the form of a parenterally acceptable aqueous solution which is pyrogen-free and has suitable pH, isotonicity and stability. Liquid pharmaceutical compositions generally include a liquid vehicle such as water. Preferably, the liquid vehicle will include a physiological saline solution, dextrose ethanol or other saccharide solution or glycols such as ethylene glycol, propylene glycol or polyethylene glycol or combinations thereof may be included. Further examples include other isotonic vehicles such as physiological salt solutions, e.g. Ringers solution or Lactated Ringer's solution.

If the inventive pharmaceutical composition comprises an aqueous solution of cells or (spherical) microcapsules as defined herein, and e.g. a buffer, said (spherical) microcapsule is typically present in the pharmaceutical composition in a concentration as defined above, e.g. of about 1×10⁵ to about 5×10⁸ cells/100 μl, about 1×10⁶ to about 5×10⁸ cells/100 μl or about 1×10⁷ cells/μl to about 5×10⁸ cells/100 μl, more preferably in a concentration of about 1×10⁵ to about 5×10⁶ cells/100 μl, about 1×10⁶ to about 5×10⁷ cells/100 μl, or about 1×10⁷ cells/μl to about 5×10⁸ cells/100 μl, and most preferably in a concentration of about 1×10⁵ to about 5×10⁶ cells/100 μl. Preferably, said pharmaceutical composition has a pH from about 2.0 to about 10.0, preferably from about 7.0 to about 8.5.

It is possible that other ingredients may be present in the inventive pharmaceutical composition. Such additional ingredients may include wetting agents, emulsifiers, antioxidants, bulking agents, pH buffering agents (e.g. phosphate or citrate or maleate buffers), preservatives, surfactants, stabilizers, tonicity modifiers, cheating agents, metal ions, oleaginous vehicles, proteins (e.g. human serum albumin, gelatin or proteins) and/or a zwitterion (e.g. an amino acid such as betaine, taurine, arginine, glycine, lysine and histidine). Such ingredients are selected by a skilled person according to the specific requirements of the cells embedded in the core of the (spherical) microcapsule, as used according to the present invention, i.e. the ingredients are not cytotoxic and ensure viability of the cells. Furthermore, such ingredients may stabilize GLP-1 peptides already encoded and secreted by the cells embedded in the core of the (spherical) microcapsule, as used according to the present invention.

With regard to buffers these are preferably selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethane, hepes, bicine, tricine, malic acid, succinate, maleic acid, fumaric acid, tartaric acid, aspartic acid or mixtures thereof. Each one of these specific buffers constitutes an alternative embodiment of the invention.

The use of all of the afore-mentioned additives in pharmaceutical compositions containing cells as defined herein and/or the (spherical) microcapsule as used according to the present invention, is well-known to the skilled person, in particular with regard to concentration ranges of the same. For convenience reference is made to Remington: The Science and Practice of Pharmacy, 19th edition, 1995.

Inventive pharmaceutical compositions containing cells, encoding and secreting GLP-1 as defined herein, and/or (spherical) microcapsules as defined herein, are preferably administered in a manner as defined herein for treatments in general. Such administrations are preferably compatible with the dosage formulation, and comprise preferably a safe and effective amount of the active ingredients as defined herein, i.e. such amount which is regarded as safe but therapeutically effective. The quantity of cells, encoding and secreting at least GLP-1 as defined herein, and preferably secreting additionally VEGF, preferably encapsulated in (spherical) microcapsules as defined herein, to be administered with an inventive pharmaceutical composition (or, if required, alone), depends on the subject and the disease to be treated, including, e.g., the severity of the patient's disease. Suitable dosage ranges depend on the amount of biologically active GLP-1 peptide secreted by the (spherical) microcapsules (as contained in the inventive pharmaceutical composition) during a predetermined time period and typically range in the order of one to several hundred micrograms (GLP-1) per day as defined herein.

In the present invention, if not otherwise indicated, different alternatives and embodiments may be combined with each other. Furthermore, the term “comprising” shall not be construed as meaning “consisting of”, if not specifically mentioned. However, in the context of the present invention, term “comprising” may be substituted with the term “consisting of”, where applicable.

### DESCRIPTION OF FIGURES

The invention is illustrated further in the accompanying Figures. However, it is not intended to limit the scope of the invention to the content of the Figures as shown in the following.

FIG. 1: shows a non-limiting overview over exemplary constructs a-m (see also Example 1), which may be contained in cells used for preparation of the (spherical) microcapsules, as used according to the present invention.

FIG. 2: depicts the results of transient expression of different GLP-1 constructs in hTERT-MSC and HEK293 cells and of active GLP-1 after transient transfection (see also Example 2). Only marginal active GLP-1 levels can be found in the monomeric GLP-1 constructs #103 and #317 (having just one copy of GLP-1(7-37)). An enormous gain in expression was observed in the dimeric GLP-1 construct #217 (having GLP-1(7-37) as component (I) and as component (III)) both in hTERT-MSC and in HEK293 cells.

FIG. 3: shows a Western Blot Analysis of a cell culture supernatant from GLP-1 secreting cells (see also Example 3). Lane 1: 100 ng synthetic GLP-1(7-37) dissolved in supernatant of mock transfected hTERT-MSC cells; Lane 2: supernatant of hTERT-MSC cells (clone 79TM217/13) secreting dimeric GLP-1 from construct #217; Lane 3: supernatant of AtT20 cells (clone 81-A-217/3) secreting dimeric GLP-1 from construct #217; Lane M: prestained protein marker [kDa]). The results show that peptides as defined herein containing GLP-1(7-37) and a C-terminal appendix (2 and 3 in FIG. 3) are secreted from the transfected cell lines and can be detected using an anti-GLP-1 antibody, which binds to the mid-molecular epitopes of GLP-1(7-37).

FIG. 4: describes plasma stability tests (in vitro) carried out with GLP-1 peptides as used according to the present invention. Therefore, HEK293 cells were transiently transfected with constructs (1) #103 GLP-1(7-37), (2) #317 GLP-1(7-37)-IP2-extended with 11 AA and (3) #217 GLP-1(7-37)-IP2-GLP-1(7-37). HEK293 cells are effective hosts for the gene construct (see also Example 4).

FIG. 5: describes a plasma stability kinetic (in vitro) carried out with supernatant of stably transfected hTERT-MSC cell clone 79TM217/18K5 secreting GLP-1 peptide CM1 produced by construct #217 GLP-1(7-37)-IP2-GLP-1(7-37) and synthetic GLP-1(7-37) as control. The results are obtained from three independent experiments. Active GLP-1 was measured using the GLP-1 (active) ELISA (Linco).

FIG. 6: shows a Western Blot for the peptides indicated below. The following values are given: SEQ ID NO: 1 (ID syn) corresponds to GLP-1(7-37), 31 aa, 3,3 kD; SEQ ID NO:8 (ID8 syn, CM3) corresponds to GLP-1(7-37)-IP2, 46 aa, 5.1 kD; SEQ ID NO: 7 (ID7rec, CM2) corresponds to GLP-1(7-37)-IP2-RR-GLP2, 83 aa, 9.4 kD; SEQ ID NO: 6 (ID6syn, CM1) corresponds to GLP-1(7-37)-IP2-RR-GLP1(7-37), 79 aa, 8.7 kD (see also Example 5).

FIG. 7: illustrates dose response curves for GLP-1 receptor mediated cAMP increase in the bioassay cell line 111 CHO349/18. Stimulation was done with serially diluted conditioned medium of 79TM217/18K5 cells secreting CM1 produced by construct #217 GLP-1(7-37)-IP2-GLP-1(7-37). No detectable cAMP response was found in the parental hMSC-TERT cell line. The graph was prepared from five independent experiments. The peptide dose that produces a half maximal effect (ED50) in the cAMP bioassay has been determined to be 353 pM (see also Example 6).

FIG. 8: illustrates characterization of cells used for (spherical) microcapsules as defined herein, after immortalising the cells in advance. As may be seen from FIG. 9 A, immortalised cells are still able to differentiate into adipocytes, osteocytes and chondrocytes as their non-immortalised counterparts (left, right). Immortalised cells have fibroblastic morphology and are more homogeneous regarding size and granularity as the mortal MSCs as shown by flow cytometry e.g. using CD 44 and CD166 epitope markers which are characteristic for the primary cells used here. Immortalised cells express the same CD markers as their non immortalised counterparts (see FIG. 9B).

FIG. 9: shows the anti-apoptotic efficacy of the C-terminal elongated GLP-1 analogue CM1. Apoptosis is induced in RIN-5F cells by addition of the protein biosynthesis inhibitor cycloheximid (CHX) in a final of 10 μg/ml and 100 μg/ml respectively. The presence of different concentrations of the recombinantly in E. coli produced dimeric GLP-1 fusion protein CM1 result in a significant (p<0.01) increase of cell viability, which is quantified after an incubation period of 24 hours.

FIG. 10: is a schematic diagram of the inventive concept using (spherical) microcapsules encoding and secreting at least GLP-1 as utilized in the treatment of vascular diseases. Cells, e.g. mesenchymal stem cells, mesenchymal stromal cells or allogeneic cells are encapsulated in a thin selectively permeable alginate matrix forming (spherical) microcapsules encoding and secreting GLP-1. The alginate matrix is permeable for oxygen and nutrients supplying the encapsulated cells, as well as for GLP-1 or the GLP-1 fusion protein encoded and secreted by the cells. On the other hand, cells and components of the immune system cannot pass this barrier as depicted herein. Left: schematic diagram of the inventive concept using (spherical) microcapsules encoding and secreting at least GLP-1 and preferably secreting additionally VEGF. The cell containing corebead (cream-coloured) is surrounded by a layer of pure alginate (grey) Right: (spherical) microcapsules encoding and secreting GLP-1 in vitro.

FIG. 11 depicts exemplary microphotographs of the bright field image (A) and vitality staining (B) of 160 μm inventive (spherical) microcapsules (CellBeads) encoding and secreting GLP-1 showing exemplary inner diameters and total diameters of these inventive (spherical) microcapsules.

FIG. 12: shows the effect of peri-adventitial application of Cellbeads® versus no treatment controls or non-stem cell containing alginate only beads on A. vein graft remodelling, B. neoadventitial angiogenesis, and, C. vessel fibrosis, at 4 weeks post grafting. Bars represent mean (S.E.M).

FIG. 13: depicts representative photomicrographs demonstrating inhibition of neointima formation and wall thickening and induction of adventitial neoangiogenesis (arrows) by administration of Cellbeads® (*) compared to no treatment controls at 4 weeks post grafting. Periadventitial application of non-stem cell containing alginate only beads (†) was associated with high rates of graft thrombosis. N, neointima, M, media.

FIG. 14: depicts representative photomicrographs demonstrating medial fibrosis and intramural MAC387 positive staining cells (arrows) in Cellbeads®, non-stem cell containing alginate only beads and no treatment controls at 4 weeks post grafting.

FIG. 15: illustrates schematically the ventral view of the abdomen and the hind legs of a mouse prepared for induction of peripheral limb ischemia. The left leg is used for ischemia induction while the right leg serves as a control. The femoral artery is ligated between the points indicated by crosses and cauterized.

FIG. 16: depicts representative photomicrographs of Hematoxylin and Eosin staining on histological sections of mice limbs after administration of Micro-CellBeads into the perivascular space around femoral artery and vein demonstrating the persistence of Micro-CellBeads. Therein FIG. 16a shows H/E staining one day after surgery, 16b shows a detailed magnification of the area of FIG. 16a that is marked by a frame. FIG. 16c shows a H/E stained limb section seven days after surgery, 16d shows a detailed magnification of the area of FIG. 16c that is marked by a frame. In FIG. 16b as well as in FIG. 16d the femoral artery, the femoral nerve, the femoral vein and the injected cell beads are indicated by arrows.

FIG. 17: depicts representative photomicrographs of histological sections of mice limbs with fluorescent immunhistochemical detection of Isolectin (green), a-smooth muscle actin (red) and DAPI-stained cell nuclei (blue) A) 7 days after administration of Micro-CellBeads upon induction of peripheral limb ischemia (shown by FIG. 17a and in a higher magnification corresponding to the area marke by a frame in FIG. 17a by FIG. 17b), B) 7 days after administration of vehicle upon induction of peripheral limb ischemia (shown by FIG. 17c and in a higher magnification corresponding to the area marke by a frame in FIG. 17c by FIG. 17d), C) within a sham control (FIG. 17e).

### EXAMPLES

The invention is illustrated further in the accompanying examples. However, it is not intended to limit the scope of the invention to the content of the Examples as shown in the following.

### Example 1

### Creation of Genetic Constructs

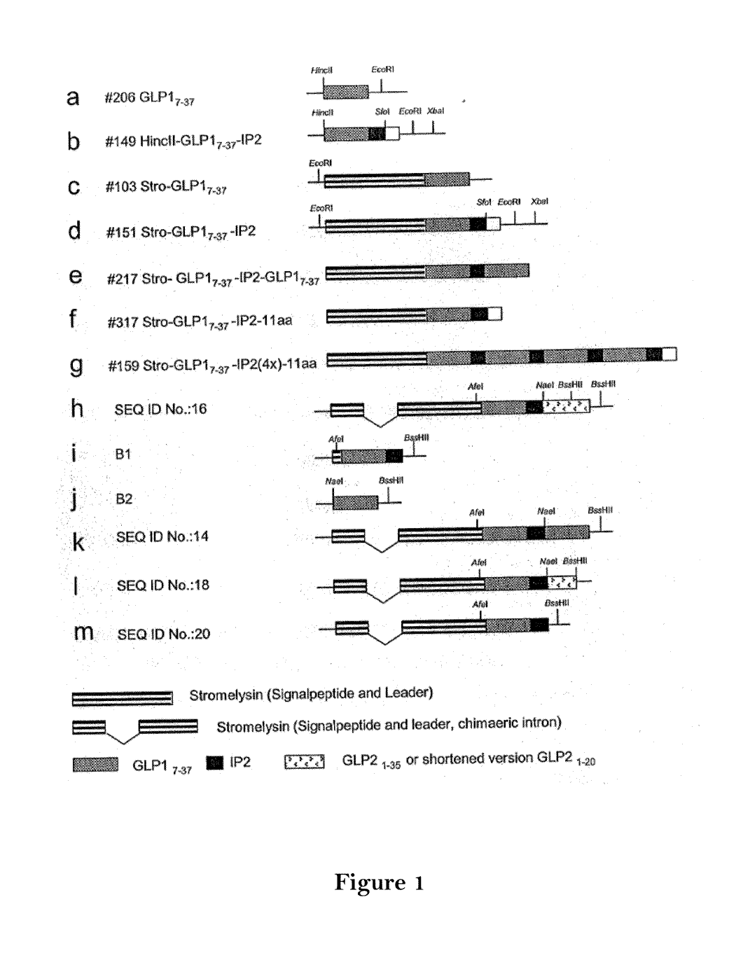

The coding sequence for GLP-1(7-37) cDNA was synthesized synthetically, in a sequence including HincII and EcoRI sites as indicated in FIG. 1a. Separately the cDNA illustrated in FIG. 1b was synthesized, including the coding sequences for GLP-1(7-37), IP2 and restriction sites for SfoI, EcoRI and XbaI, as illustrated in FIG. 1b. To direct GLP-1 to the secretory pathway, the heterologous signal sequence of stromelysin 3 (Acc. No. NM_{—}005940) was used. Therefore the cDNA, encoding stromelysin signal and leader sequence was reverse transcriptase PCR amplified from human RNA, and used with the construct of FIG. 1a or FIG. 1b to form the construct shown in FIG. 1c and FIG. 1d, respectively.

The HindI/EcoRI fragment of the FIG. 1a construct is cloned into the SfoI site of the sequence of FIG. 1d to form the construct FIG. 1e. Similarly, the EcoRI fragment of FIG. 1d is cloned into the EcoRI site of an eukaryotic expression plasmid, to produce the construct shown in FIG. 1f. To form the construct shown in FIG. 1g, the HindII/XbaI fragment of the construct shown in FIG. 1b is repetitively cloned into the SfoI/XbaI site of the construct shown in FIG. 1d. FIG. 1h shows a synthesized, codon optimized sequence encoding the stromelysin leader and signal sequences interrupted by a shortened endogenous intron sequence, fused to sequences encoding human GLP-1(7-37), IP2 and GLP-2(1-35). The DNA sequence of the construct FIG. 1h is SEQ ID NO: 16, while SEQ ID NO: 15 also shows the sequence of the translated peptide.

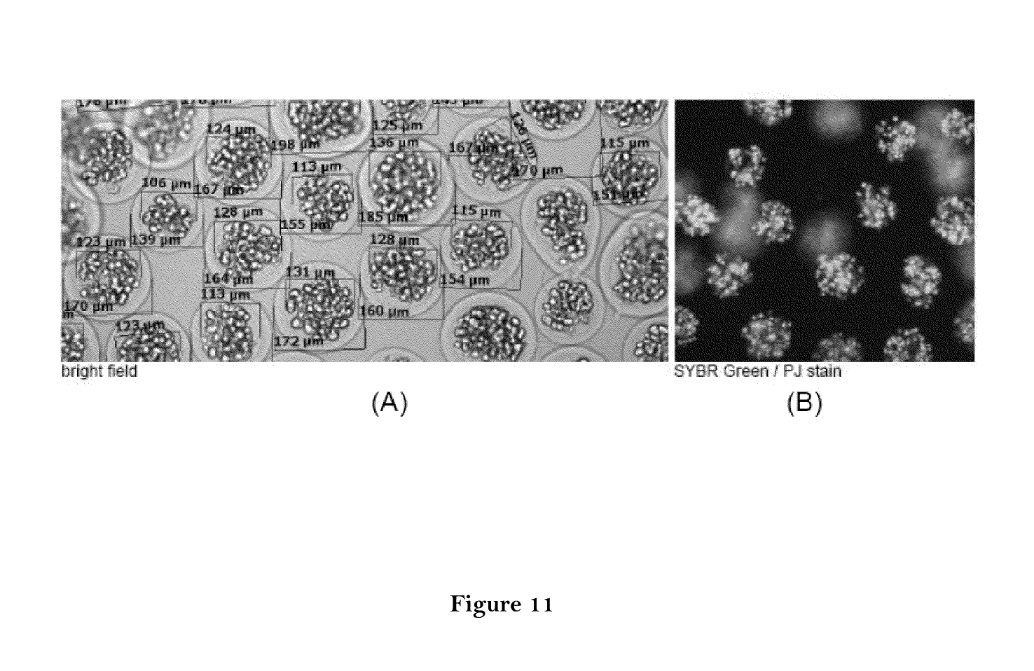

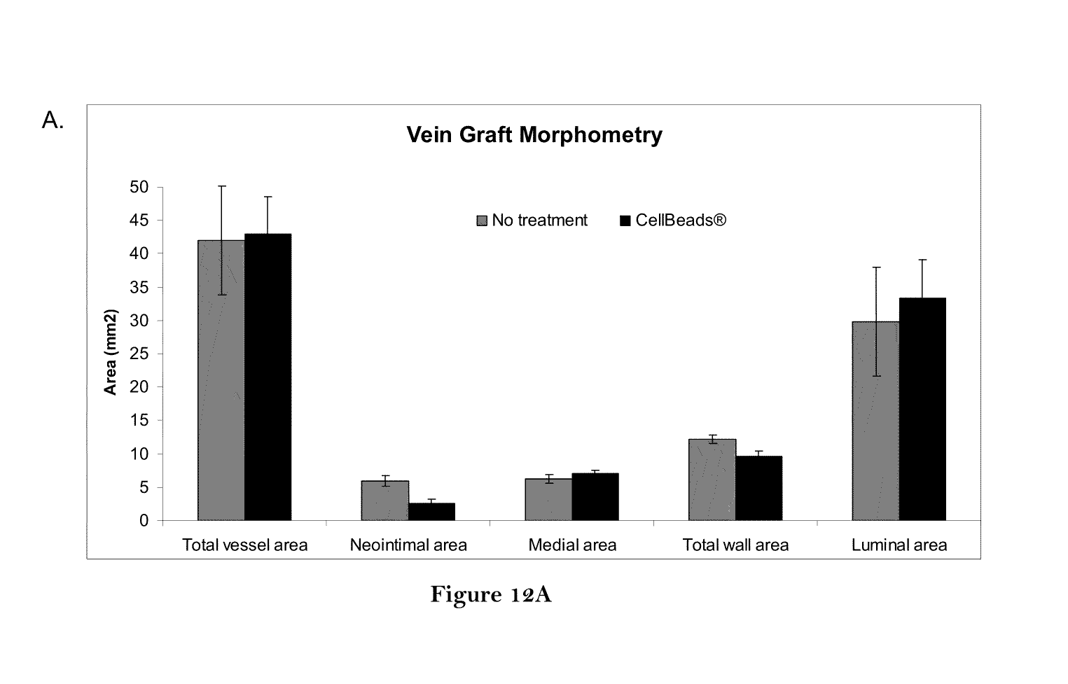

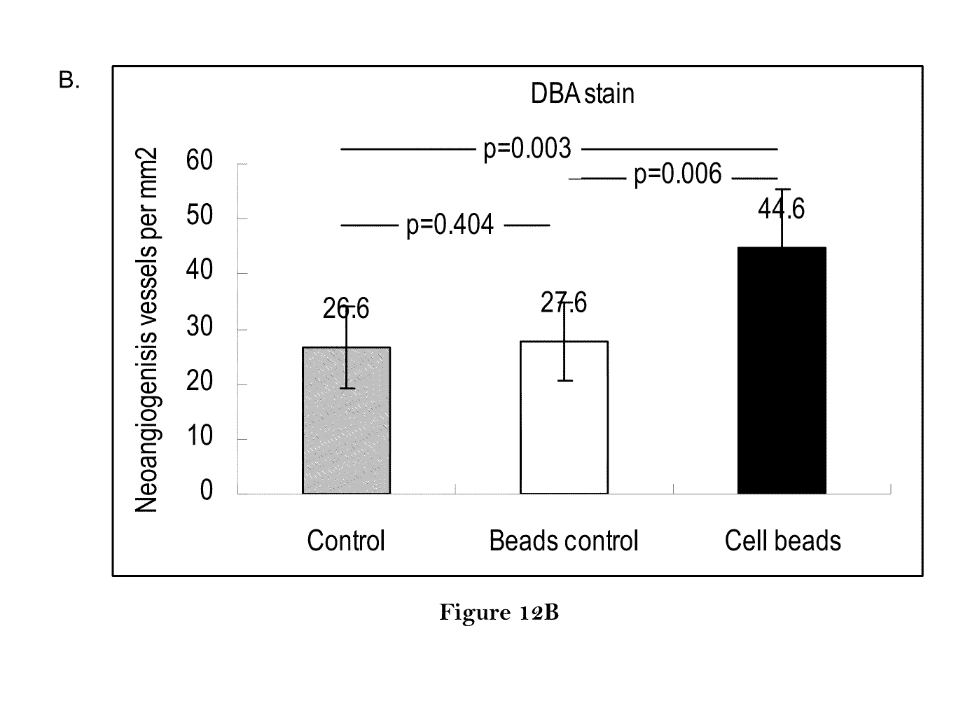

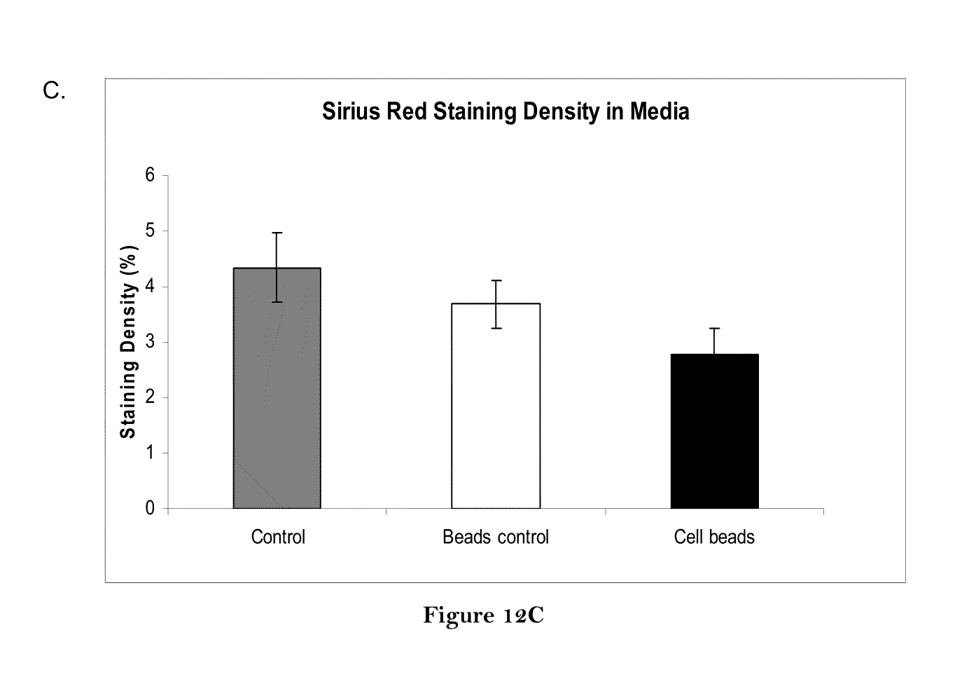

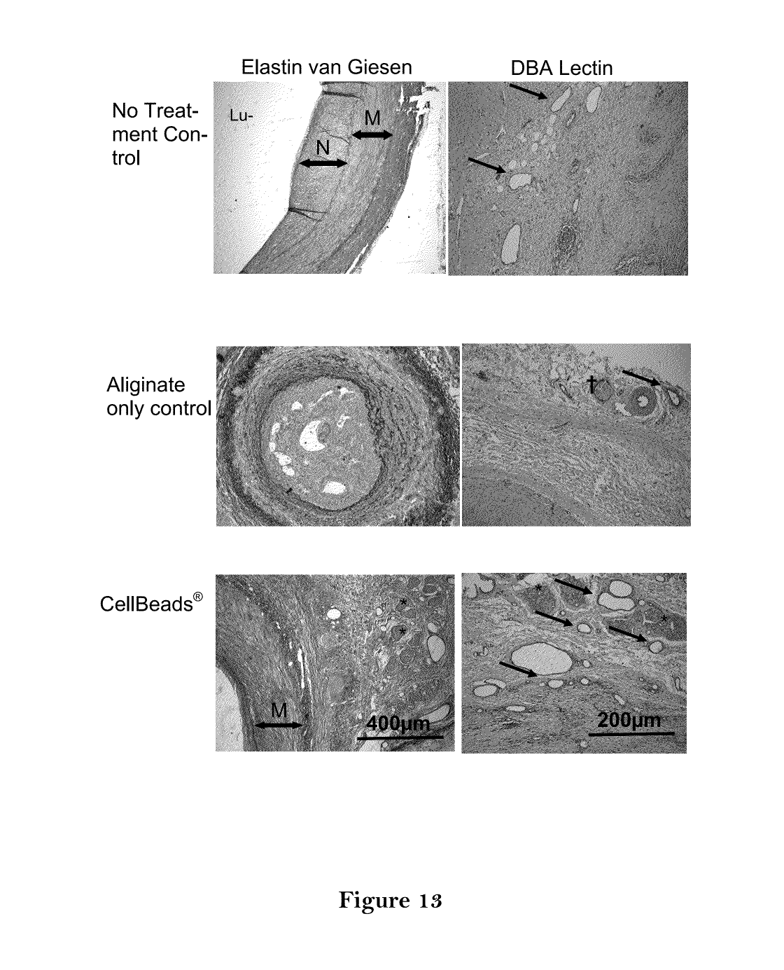

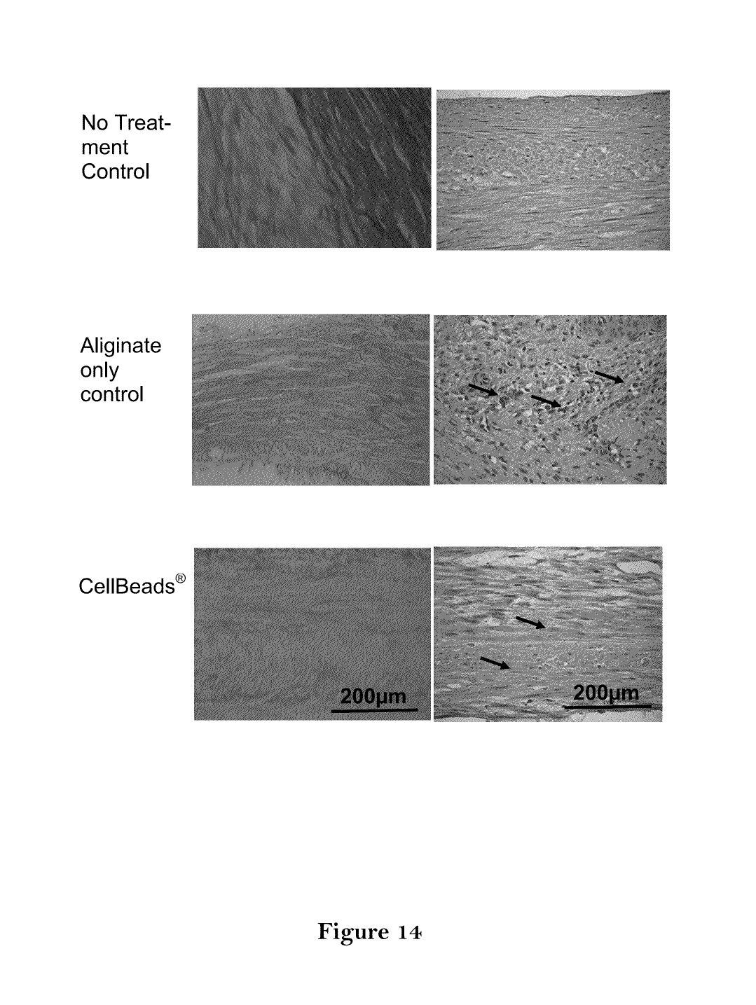

Also synthesized are the sequences in FIGS. 1i and 1j. These are then used to form the construct in FIG. 1k, by cloning the NaeI/BssHII fragment of FIG. 1j into the NaeI/BssHII linearised sequence of FIG. 1h. The DNA sequence of the construct FIG. 1k is SEQ ID NO: 14, while SEQ ID NO: 13 also shows the sequence of the translated peptide. The construct of FIG. 11 is formed by BssHII digest and religation of the sequence of FIG. 1h. The DNA sequence of the construct FIG. 1l is SEQ ID NO: 18, while SEQ ID NO: 17 also shows the sequence of the translated peptide. The construct of FIG. 1m is formed by cloning the AfeI/BssHII fragment of the sequence of FIG. 1i into the AfeI/BssHII linearised sequence of FIG. 1 h. The DNA sequence of the construct FIG. 1m is SEQ ID NO: 20, while SEQ ID NO:19 also shows the sequence of the translated peptide.

The herein described constructs may be made by a person skilled in the art using routine techniques.

### Example 2

### Transfection, Clonal Selection and GLP-1 Expression of Mammalian Cells

Source of the cells: HEK293 (human embryonic kidney cell line, # ACC 305, DSMZ Cell Culture Collection, Germany), AtT20 (Mouse LAF1 pituitary gland tumour cell line, #87021902, European Cell Culture Collection, UK), hTERT-MSC cells are generated and provided by Prof. Kassem, University Hospital of Odense, Denmark.

For transfection of 10⁶ cells 0.5-2 μg plasmid DNA with different GLP-1 constructs was used. The constructs were generated as described in Example 1. HEK293 cells were transfected by standard calcium phosphate co-precipitation method as described in Current Protocols in Molecular Biology (Ausubel et al. 1994ff Harvard Medical School Vol2., Unit 9.1). AtT20 cells were transfected using FuGene (Roche) as described in Current Protocols in Molecular Biology (Ausubel et. al. 1994ff, Harvard Medical School Vol 2., Unit 9.4). Transfection of hTERT-MSC cells was performed using the Nucleofector technology (Amaxa), a non-viral method which is based on the combination of electrical parameters and cell-type specific solutions. Using the Nucleofector device (program C17) and the Nucleofector solution VPE-1001 transfection efficiencies >60% have been achieved. 48 hours after transfection selection of cell clones with stable integration of DNA into the chromosome was performed by adding the selective agent blasticidin (2 μg/ml) into the culture medium. 12-15 days later, stable transfected cell clones could be isolated and expanded for characterization.

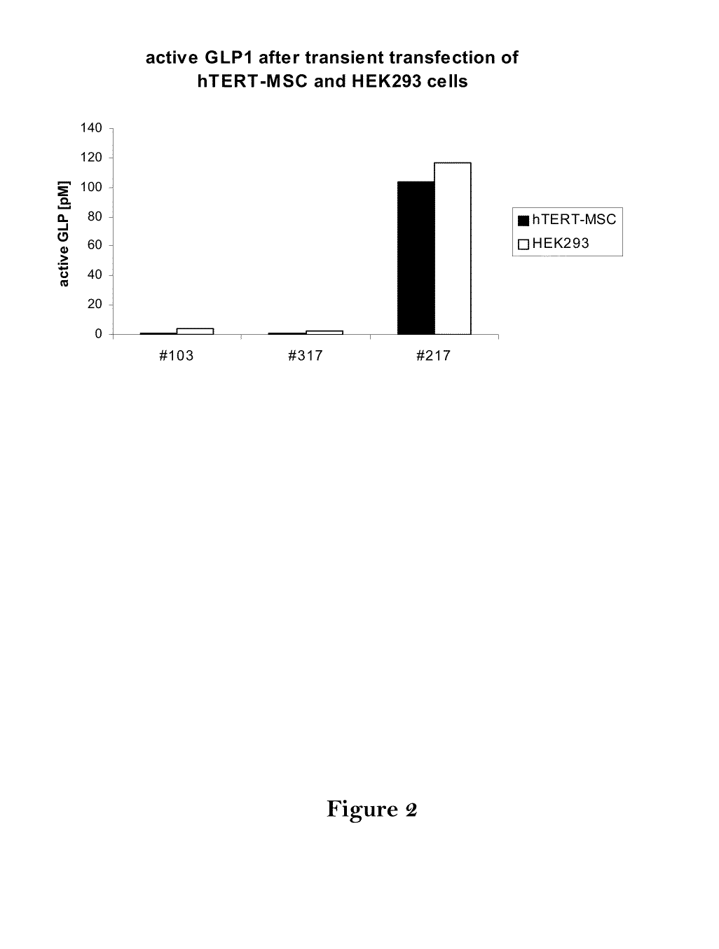

Transient expression of different GLP-1 constructs was measured in hTERT-MSC and HEK293 cells. Whereas only marginal active GLP-1 level can be found in the monomeric GLP-1 constructs #103 and #317 (having just one copy of GLP-1(7-37) an enormous gain in expression can be found in the dimeric GLP-1 construct #217 (having GLP-1(7-37) as component (I) and as component (III)) both in hTERT-MSC and in HEK293 cells. Results are summarized in FIG. 2. An elongation of the construct to the GLP-1 construct #159 (having four IP2 copies as component (II)) results in no further significant increase (not shown). After transfection of hTERT-MSC cells with different constructs clones were selected, which stably express GLP-1. The expression levels are shown in Table 1.

TABLE 1

| | | active GLP per 10⁶ |
|---|---|---|
| | | cells and hour |
| construct | cell clone | [pmol] |
| #103 GLP1₍₇₋₃₇₎ | 49TM113/13 | 0.4 |
| #317 GLP1₍₇₋₃₇₎-IP2-11aa | 71TM169/1 | 0.6 |
| #217 GLP1₍₇₋₃₇₎-IP2-GLP1₍₇₋₃₇₎ | 79TM217/13 | 2.7 |

### Example 3

### Western Blot Analysis of GLP-1 Peptides, Secreted from Mammalian Cells

Cell culture supernatant from GLP-1 secreting cells was separated in a 10%-20% gradient SDS PAGE (120V, 90 minutes) and transferred to a PVDF membrane (Immobilon-P Membrane 0.45 Millipore IPVH 00010) by semi-dry blotting (2.0 mA/cm2, 60 minutes). After methanol fixation and blocking (3% (w:v) BSA, 0.1% (v:v) Tween-20 in TBS) the membrane was immunoblotted with 1 μg/ml anti-GLP-1 antibody (HYB 147-12, Antibodyshop) at 4° C. o/n. After washing and incubation with 0.02 μg/ml detection antibody (Anti Mouse IgG, HRP conjugated, Perkin Elmer PC 2855-1197) at RT for 4 hours, chemiluminescence detection reveals the location of the protein.

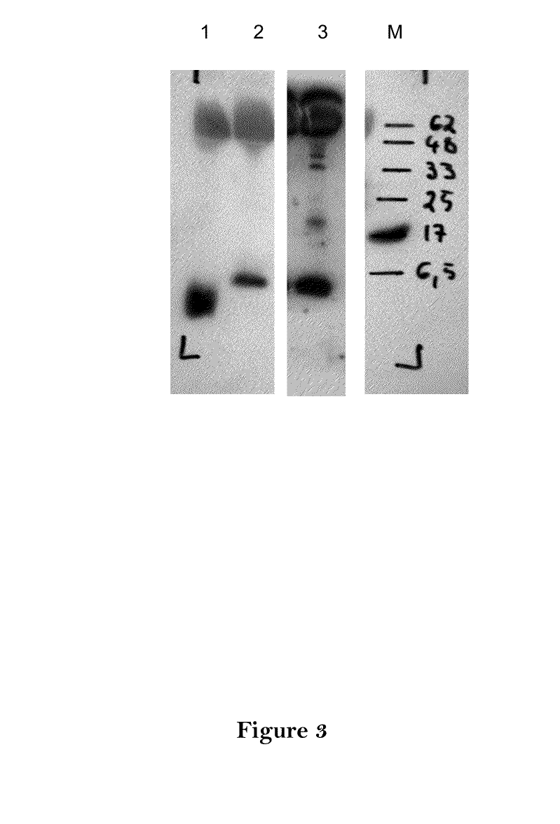

Western Blot Analysis is shown in FIG. 3 (1: 100 ng synthetic GLP-1(7-37) dissolved in supernatant of mock transfected hTERT-MSC cells, 2: supernatant of hTERT-MSC cells (clone 79TM217/13) secreting dimeric GLP-1 from construct #217, 3: supernatant of AtT20 cells (clone 81-A-217/3) secreting dimeric GLP-1 from construct #217; M: prestained protein marker [kDa]). The results show that peptides containing GLP-1(7-37) and a C-terminal appendix (2 and 3 in FIG. 3) are secreted from the transfected cell lines and can be detected using an anti-GLP-1 antibody, which binds to the mid-molecular epitopes of GLP-1(7-37).

### Example 4

### In Vitro Plasma Stability of GLP-1 Peptides Secreted from Human Cells

HEK293 and hTERT-MSC cells were transfected with constructs, encoding the heterologous stromelysin signal sequence, which is linked to GLP-1 variants encoding the following peptides:
1: GLP-1(7-37) (construct #103)
2: GLP-1(7-37)-IP2-extended with 11 AA (construct #317)
3: GLP1(7-37)-IP2-GLP1(7-37) (construct #217)

Cell culture supernatant, containing GLP-1 peptides secreted from cells or synthetic GLP-1(7-37) (Bachem) was incubated with human lymphocyte enriched plasma containing dipeptidylpeptidase activity at 37° C. and 5% CO₂, for 3 or additionally 6 and 9 hours. Synthetic GLP-1(7-37) in supernatant from mock transfected cells was used as a positive control for DPP-IV activity, which was shown to be inhibited by addition of a DPP-IV inhibitor (#DPP4, Biotrend). Active GLP was measured using the GLP-1 (Active) ELISA (#EGLP-35K, Biotrend), using an antibody which binds to the N-terminal epitope of GLP-1(7-37) discriminating the DPP-IV degraded, inactive GLP-1(9-37) peptide.

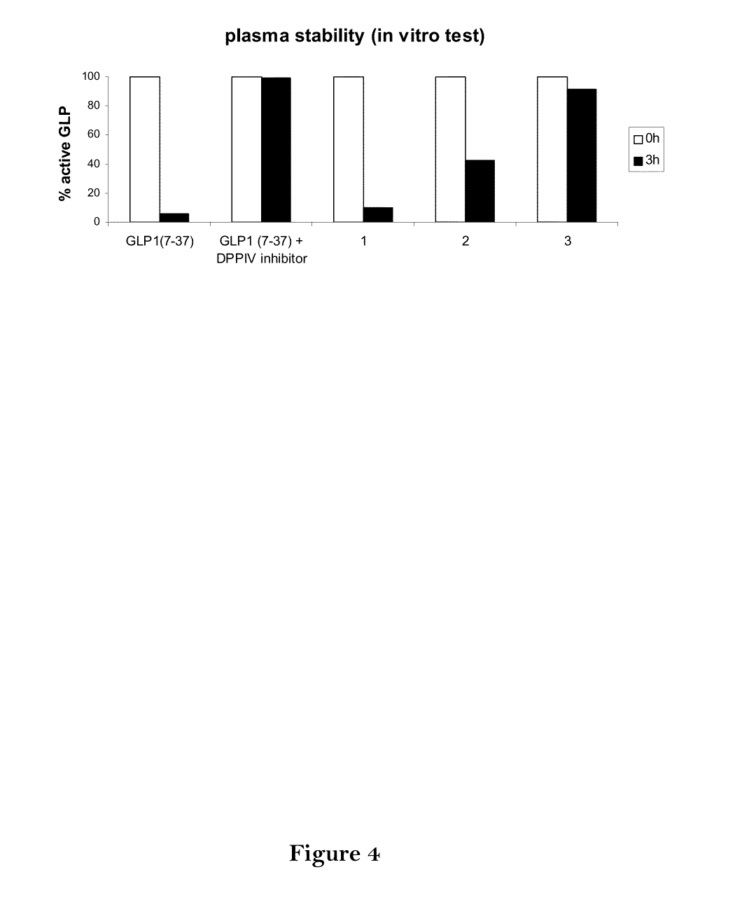

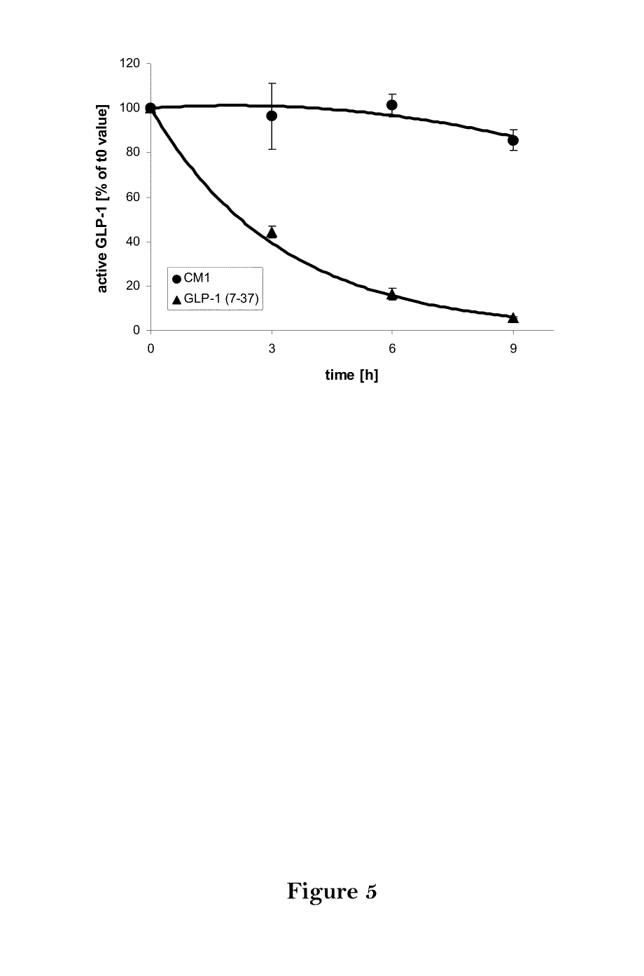

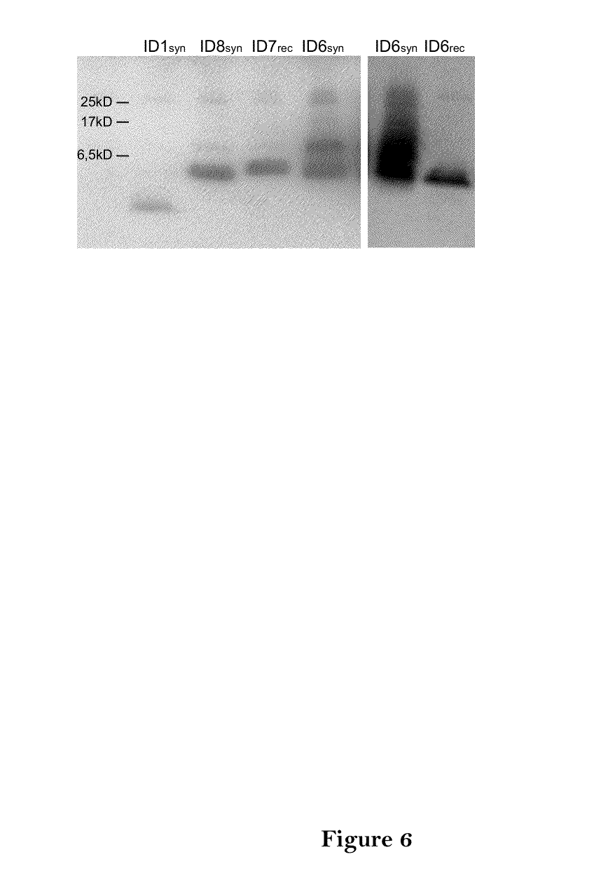

The results are shown in FIG. 4 (HEK293 cells) and 5 (hTERT-MSC cells). HEK293 and hTERT-MSC cells are both effective hosts for the gene construct. The numbering of the results for the transfected cells is 1: supernatant of cells secreting GLP-1(7-37) from construct #103, 2: supernatant of cells secreting GLP-1 extended by IP2 and 11 aminoacids from construct #317, 3: supernatant of cells secreting dimeric GLP-1 from construct #217. While construct 1 produces wild type GLP-1 which is inactivated by DPP-IV in a similar way to synthetic GLP-1, the C-terminally elongated GLP-1 forms (2 and 3 in FIGS. 4, 3 in FIG. 5) are more resistant to degradation. The C-terminal extended GLP-1 peptides are significantly stabilized in human plasma in vitro. The peptide with the dimeric GLP-1 sequence (3) is nearly fully stabilized to DPP-IV degradation in vitro.

### Example 5

### In Vitro Bioactivity of GLP-1 Peptides Measured by cAMP Release

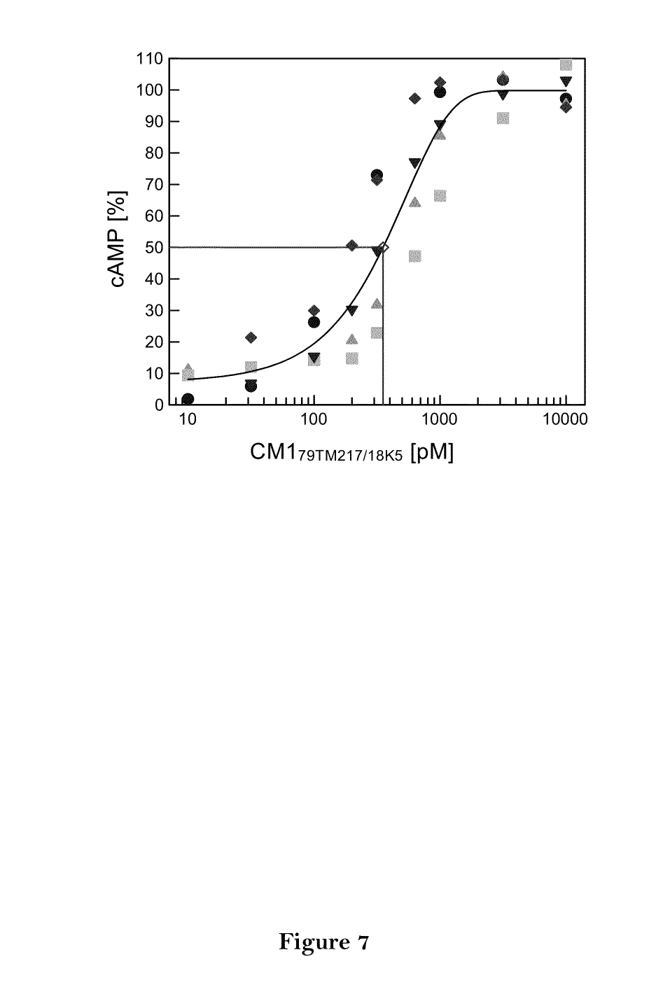

GLP-1(7-37) exerts its biological actions through the seven-transmembrane-spanning, G protein coupled GLP-1 receptor, which leads to activation of protein kinase A signalling through the second messenger cyclic AMP. To ensure that the C terminal elongation of CM1 does not interfere with GLP-1's mode of action, CM1 bioactivity was quantified in an in vitro bioassay, which determines cAMP increase in a GLP-1 receptor expressing cell line after incubation with different concentrations of the peptide. The GLP-1 receptor expressing cell line used for the study (clone 111CHO0349/18) is a CHO (chinese hamster ovary) cell line stably transfected with the human GLP-1 receptor. The dose response curves for CM1 produced in the 79TM217/18K5 cells outline the bioactivity of the peptide is shown in FIG. 7. The peptide dose that produces a half maximal effect (ED50) in the cAMP bioassay has been determined to be 353 μM.

### Example 6

### In Vitro Human Plasma Stability of GLP-1^{CM} Peptides

Synthetic GLP-1 peptides (SEQ ID NO: 1_{syn}, SEQ ID NO: 6_{syn}, SEQ ID NO: 7_{rec}, SEQ ID NO: 8_{syn}) were incubated at concentrations of 20 ng/ml with human plasma at 37° C. and 5% CO, for 3 hours. Dipeptidylpeptidase activity of the plasma was inhibited by a DPP-IV inhibitor (#DPP4, Biotrend). Active GLP was measured using the GLP-1 (Active) ELISA (#EGLP-35K, Biotrend).

In contrast to the native GLP-1₍₇₋₃₇₎ (SEQ ID NO: 1) the C-terminally elongated GLP-1 peptides SEQ ID NO:6, SEQ ID NO:7, and SEQ ID NO:8 are significantly stabilized in human plasma in vitro (FIG. 7). As control (on the right hand side) the results obtained for experiments with addition of DPP-IV inhibitor are shown. GLP-1 activity is completely maintained in these control experiments.

### Example 7

### Plasmid Creation

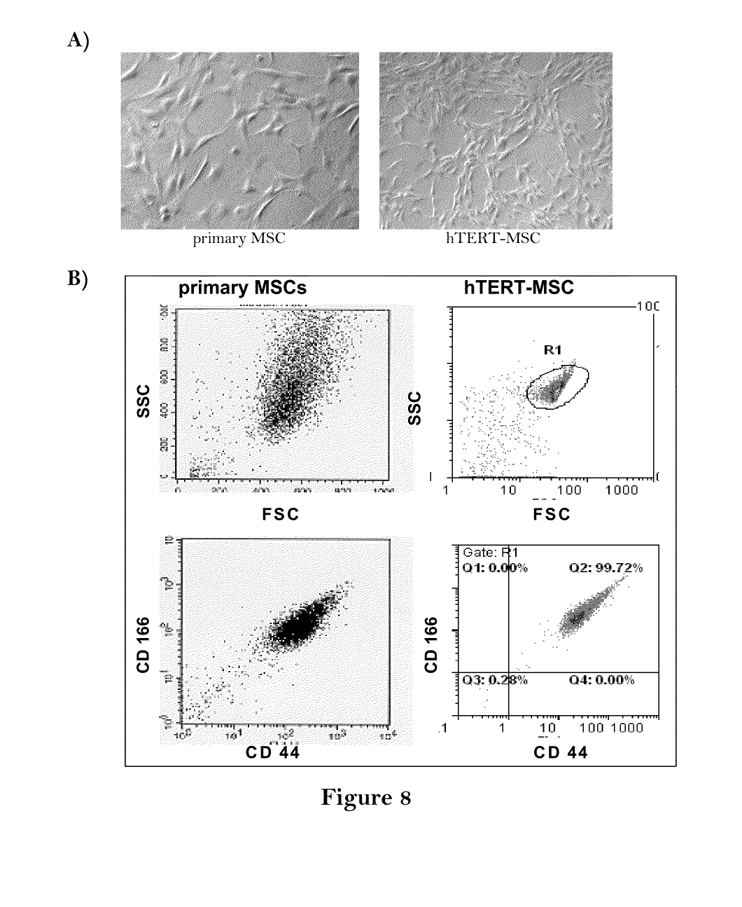

The vector for transient and stable gene expression consists of two separate transcription units, one for the gene of interest (GOI) and one for the fusion of the suicide gene HSV thymidine kinase and the resistance gene blasticidin. For the first transcription unit, the human ubiquitin B promoter was used, and for the second transcription unit the human ferritin promoter was used. The plasmid is based on plasmid pCM4, having 7,919 base pairs, shown schematically in FIG. 8.

As shown in FIG. 8, transcription unit 1, comprises the following components:
CMVenh: immediate early enhancer human cytomegalovirus
ubiB human: ubiquitin promoter B
Stro-GLP: fusion gene, coding for signal peptide and leader sequence of stromelysin and GLPI constructs
ori pMBI: E coli minimal origin of replication.
Hygro: hygromycin B resistance gene.

### Transcription Unit 2,

SV 40 enh: SV40 enhancer.
FerH: Human ferritin H promoter combined with 5′UTR of the murine EFI gene.
Tk-bla: fusion gene coding for herpes simplex virus type 1 thymidine kinase and blasticidine resistance gene.

For transient expression the circular plasmid was used. For the selection of stable expressing cell clones, the plasmid was linearised and bacterial sequences (pMB1 origin and hygromycin gene) eliminated.

### Example 8

### Production of Mesenchymal Stem Cell Lines or Mesenchymal Stromal Cell Lines (MSC).

The mesenchymal stem cell line was generated by Prof. Kassem, University Hospital of Odense, Denmark (published in Simonsen et al., 2002, Nature Biotechnology 20m, 592-596) according to following criteria:

### Origin

The production cell line consists of mesenchymal stem cells (MSC), isolated from bone marrow aspirates of a healthy male donor (age 33).

### Immortalisation

Cells were immortalised by introduction of the coding sequence of the telomerase reverse transcriptase. Retroviral transduction was performed by packaging the GCsam retroviral vector in which the expression of the transgene is driven by the Moloney murine leukaemia virus long terminal repeat in PG13. Transduction was performed on day 9 (PDL 12) of culture. The cell line has so far been cultivated until population doubling level (PDL) of 260.

The insertion locus was tested by fluorescence in situ hybridization and southern blot. There is only one insertion locus of ecotopic hTERT on chromosome 5 (5q23-31). Analysis was performed at PDL 186. Giemsa banding and comparative genomic hybridization revealed that hMSC-TERT did not develop any numerical or structural chromosomal abnormalities at PDL 96 and maintained a normal diploid male karyotype. Tumourigeneity was tested in immunodeficient mice after subcutaneous implantation for six months and was found negative for PDL 80.

### Flow Cytometry (FACS) Analysis

Cells were cultured in standard growth medium to 80% confluence. Cells were trypsinised and assayed for size and granularity by FACScan flow cytometer (Becton-Dickinson). For surface marker studies typsinised cells were stained with antibodies directly conjugated to a fluorescent dye (FITC-conjugated mouse anti human CD44 monoclonal antibody, #CBL154F, Cymbus Biotechnology; phycoerythrin-conjugated mouse anti human CD166 monoclonal antibody, #559263, BD Pharmingen) for 30 min on ice. Samples were washed and fixed with 1% of paraformaldehyde until analysis with FACScan (Becton-Dickinson).

### Characterization

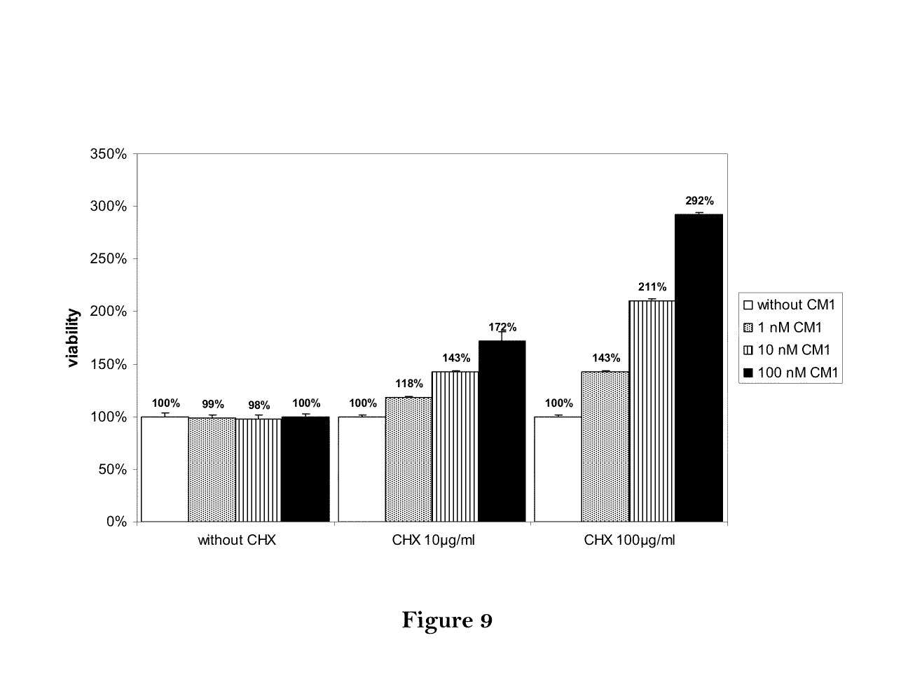

Immortalised cells are still able to differentiate into adipocytes, osteocytes and chondrocytes as their non-immortalised counterparts (see FIG. 9A). Immortalised cells have fibroblastic morphology and are more homogeneous regarding size and granularity as the mortal MSCs as shown by flow cytometry e.g. using CD 44 and CD166 epitope markers which are characteristic of the primary cells used here. Immortalised cells express the same CD markers as their non immortalised counterparts (see FIG. 9B).

### Cultivation

Serum containing medium: 7% Earles MEM
   10% FCS
   2 mM L-Glutamine
   1 mM Sodiumpyruvate
   100 U/ml Penicillin
   0.1 mg/ml Streptomycin

The population doubling is between 26 and 30 hours.

### Transfection and Clonal Selection

For transfection of 10⁶ cells 0.5-2 μg plasmid DNA with different GLP1 constructs was used. HEK293 cells were transfected by standard calcium phosphate co-precipitation method. AtT20 cells were transfected using FuGene (Roche).

Transfection of hTERT-MSC cells was performed using the Nucleofector technology (amaxa), a non-viral method which is based on the combination of electrical parameters and cell-type specific solutions. Using the Nucleofector device (programme C17) and the Nucleofetor solutionVPE-1001 transfection efficiencies >60% have been achieved.

48 hours after transfection selection of cell clones with stable integration of DNA into the chromosome was performed by adding the selective agent blasticidin (2 μg/ml) into the culture medium. 12-15 days later, stable transfected cell clones could be isolated and expanded for characterization.

### Expression

Transient expression of different GLP constructs was measured in hTERT-MSC and HEK293 cells. An active GLP1 level can be found in the monomeric GLP1 constructs #103 (Stro-GLP1₍₇₋₃₇₎) and #317 (Stro-GLP1₍₇₋₃₇₎-IP2-extended with 11aa) and an enormous gain in expression can be found in the dimeric GLP1 construct #217 (Stro-GLP1₍₇₋₃₇₎-IP2-GLP1₍₇₋₃₇₎) both in hTERT-MSC and in HEK293 cells. An elongation of construct #317 to the tetrameric GLP1 construct #159 (Stro-GLP1₍₇₋₃₇₎-IP2 (4x)-11aa) results in an similar activity (see also herein FIG. 2). After transfection of hTERT-MSC cells with different constructs clones were selected, which stably express GLP1 (see herein FIGS. 4 and 5, Example 4).

### Example 9

### Encapsulation

The cultivated cells to be encapsulated were washed with PBS (PAA, Austria) and separated using trypsin/EDTA (PAA, Austria). The reaction was quickly stopped using medium (dependent on cell type, for example RPMI, PAA, Austria) and the cell suspension centrifuged off (8 min at 1,200 rpm) The pellet was resuspended in PBS and the cell count determined. The desired quantity of 4×10⁷ cells was centrifuged off again (8 min at 1,200 rpm). The PBS was then completely removed by suction and 50 μl pellet was resuspended without air bubbles in 50 μl 0.9% saline buffered by 5 mM 1-histidine to a pH of 7.4. This cell suspension was taken up in 900 μl of 1.5-1.7% (w/v) sodium alginate solution (an alginate with a viscosity of approximately 5 mPa·s of 0.2% (w/v) aqueous solution at room temperature was used).

To mix the resuspended cells with the alginate solution, the solution was drawn up in a 1 ml syringe with cannulas and homogeneously mixed with the cells by way of repeated slow drawing up and drawing off. A cell concentration of 4×10⁷ cells/ml resulted.

For producing the microcapsules with a diameter of about 200 μm, a cannula with an internal diameter of 120 μm was used in an air-charged spray nozzle. An air ring with an opening of 2.0 mm was screwed over the inner cannula. The device is an adapted version of the device described in WO 00/09566. The homogeneous cell/alginate solution mixture was dripped through the described spray nozzle. For this purpose, the 1 ml syringe containing the mixture was placed on the cannula by means of a luer connector. The cell/alginate solution mixture was pressed through the cannula at a speed of 50 μl/min. The airflow was conveyed though the outer air ring at a speed of 2.5 l/min. The resulting microcapsules precipitated into a barium-containing precipitation bath (20 mM BaC1, 5 mM L-histidine, 124 mM NaCl, pH 7.0±0.1, 290 mOsmol±3) which was constructed approximately 10 cm below the spray nozzle. After a dwell time of 5 min in the barium-containing precipitation bath the microcapsules were washed five times with 20 ml PBS in each case.

500 μl of the single-layer microcapsules were then taken up in 500 μl of a 1.5-1.7% (w/v) alginate solution the same as used for the core, herein and homogeneously mixed. This suspension was taken up in a 1 ml syringe and connected by means of a luer connector to the inner channel (internal diameter: 200 μm) of the spray nozzle and pressed at a speed of 50 μl/min therethrough. A 5 ml syringe with a 1.5-1.7% alginate solution was connected by means of a luer connector to the second inner channel (internal diameter: 700 μm) and pressed there through at a speed of 250 μl/min. The airflow was conveyed through the outer air ring at a speed of 2.9 l/min. The resultant microcapsules precipitated into a barium-containing precipitation bath (20 mM BaCl, 5 mM L-histidine, 124 mM NaCl, pH 7.0 I 0.1, 290 mOsmol±3) which is constructed approximately 10 cm below the spray nozzle. After a dwell time of 5 min in the barium-containing precipitation bath, the microcapsules were washed four times with 20 ml PBS in each case and once with medium. Two-layer microcapsules with a total diameter of approximately 180-200 μm (including the alginate layer) were produced by this process, wherein the diameter of the inner, cell containing core is 120-150 μm.

The concentration of cell in the core is about 4×10⁷ cell/ml alginate. This results in (spherical) microcapsules (CellBeads) with a bead volume of 0.002-0.004 μl containing approximately 100 cells per bead. A (spherical) microcapsule encoding and secreting GLP-1 produces on average 0.2 fmol active GLP-1 per hour.

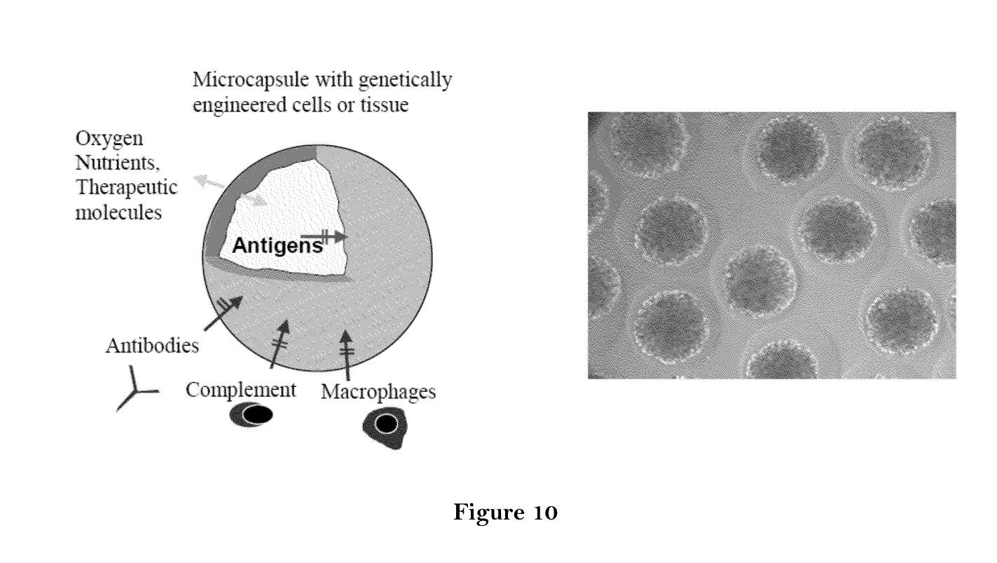

A micrograph of (spherical) microcapsules (CellBeads) containing encapsulated GLP-1 secreting hTERT-MSC cells in the core are shown in FIG. 10.

### Example 10

### Anti-Apoptotic Efficacy of C-Terminally Elongated GLP-1

The cytoprotective efficacy of the C-terminally elongated GLP-1 analogue CM1 was tested in vitro using the rat insulinoma cell line Rin-5F. 40.000 Rin-5F cells were seeded per 96 well and cultivated for 2 days in RPMI supplemented with 1% L-Glutamin and 10% fetal calf serum. Apoptosis is induced after change to serum free conditions (RPMI supplemented with 1% L-Glutamin) by addition of the protein biosynthesis inhibitor cycloheximid (CHX) in the presence of different concentrations of the recombinantly in E. coli produced dimeric GLP-1 fusion protein CM1. After 24 hours cell viability is quantified using AlamarBlue. A significant anti-apoptotic effect (p<0.01) was observed already in the presence of 1 nM GLP-1 analouge CM1. The results are given in FIG. 10.

### Example 11

### Cytokine Profile of the GLP-1 Producing hTERT-MSC Cell Line

To investigate GLP-1 independent, cytoprotective effects, the GLP-1 secreting cell line 79TM217/18K5 cell line was examined for the secretion of cytokines, chemokines and growth factors.

The cell line originates from a human stromal cell and therefore secretes a characteristic cytokine profile. A multiplex assay kit (Biosource Cytokine 30-plex) was used for measuring the 30 most abundant human cytokines, chemokines and growth factors simultaneously. No expression was found regarding the cytokines IL-1RA, IL-1β, IL-2, IL-2R, IL-4, IL-5, IL 7, IL-10, IL-12(p40/p70), IL-13, IL-15, IL-17, IP-10, EGF, Eotaxin, FGF-basic, IFN-a, IFNγ, GM CSF, G-CSF, HGF, MIG, MIP-b, MIP-1α, RANTES and TNFα (detection limit of each analyte 20 pg per 10⁵ cells and 24 h). The cytokines, which are expressed at detectable levels are summarized in table 1.

Table 1: Expression level of growth factors Vascular endothelial growth factor (VEGF), neurotrophin-3 (NT-3), glial cell line-derived neurotrophic factor (GDNF) and the cytokines Interleukin 6 (IL-6), Interleukin 8 (IL-8) and Monocyte chemotactic protein 1 (MCP-1). The factors have been quantified in cell culture supernatant of the CM1 secreting cell line 79TM217/18K5 using the VEGF ELISA (#ELH-VEGF-001; RayBio), NT-3 ELISA (#TB243, Promega), GDNF ELISA (#TB221, Promega) and the human IL-6, IL-8 and MCP-1 ELISA Kits (RayBio).

| Growth factor/Cytokine | |
|---|---|
| [pg/10⁶ cells and hour] | 79TM217/18K5 |
| VEGF | 973.0 ± 78.3 |
| NT-3 | 20.9 ± 3.5 |
| GDNF | 10.7 ± 1.5 |
| IL-6 | 378.4 ± 4.0 |
| IL-8 | 3608.7 ± 53.8 |
| MCP-1 | 16.8 ± 0.1 |

### Example 12

### Treatment of Vein Graft Disease Models

In this experiment, peri-adventitial application of inventive microcapsules (CellBeads®) at a dose of 20,000 cell.cm⁻² to porcine saphenous vein to carotid artery interposition grafts resulted in 57% reduction in neointimal area (mean difference 3.54 mm², 95% CI 1.14-5.94, p=0.008) and a 21% reduction in total wall area (mean difference 2.71 mm², 95% CI 0.40-5.00, p=0.025) relative to untreated controls. There was a 68% increase in vein graft adventitial neoangiogenesis in the Cellbead® treated group compared to untreated grafts (mean difference 18.0 vessels/mm² 95% CI 5.49 to 30.53, p=0.004). Non-stem cell containing alginate only beads appeared to cause significantly reduced graft patency. One graft from eight (12.5%) in the Cellbead® group was occluded at harvest compared to 7 out of 8 (87.5%) in the alginate only group and none in the no treatment group (p<0.0001).

### Methods:

### Animals

A total of 12 large white/landrace cross pigs weighing 28.2±1.0 Kg were used. All procedures had local ethical approval, were performed under UK government licence (Animals (Scientific Procedures) Act 1986), and conform to the Guide for the Care and Use of Laboratory Animals published by the US National Institutes of Health (NIH Publication No. 85-23, revised 1996).

### Intervention

Inventive microcapsules (CellBeads®) were prepared and stored at −80° C. Immediately prior to grafting inventive microcapsules (CellBeads®) containing approximately 500,000 cells were washed in PBS buffer at room temperature. This gel was applied to the external surface of the grafts using a syringe after completion of the distal anastomosis. Grafts with application of non-stem cell containing alginate microbeads and grafts receiving no treatment (each n=8) served as controls.

Porcine Autologous Saphenous Vein into Carotid Artery Interposition Grafts

The method of the saphenous vein-carotid interposition-grafting model has been described previously (see Rajathurai T, Rizvi S I, Lin H, Angelini G D, Newby A C, Murphy G J. Peri-adventitial rapamycin eluting microbeads promote vein graft disease in long-term pig vein-into-artery interposition grafts. Circulation: Cardiovascular Interventions 2010; 3:157-65). Animals were anaesthetised with ketamine (Ketaset 100 mg/ml) and halothane, intubated, and allowed to spontaneously ventilate. The long saphenous vein was harvested from the hind leg, the animal was heparinised by intravenous administration of 100 IU/kg of heparin (CP Pharmaceuticals Ltd, Wrexham, UK) and a 3 cm length of vein grafted as an interposition graft to the internal carotid artery using continuous 7/0 Surgipro (Auto Suture, Dagford, UK) sutures bilaterally. Inventive microcapsules (CellBeads®) or control interventions were allocated to either the right or left vein graft immediately prior to implantation. Animals were recovered, returned to their pen and fed a normal chow diet for the duration of the experiment. Grafts were harvested at 4 weeks. Only patent grafts were used for morphometry analyses.

### Histological Methods

Vein-grafts were pressure fixed at 100 mm Hg with 4% Formalin in PBS, wax embedded and sectioned into 4 μm transverse sections. Four transverse sections at equally spaced intervals along the graft length were stained with Miller's elastic van Gieson stain (EVG). For each section, the luminal margin, internal and external elastic laminae were identified and traced from digital images, and total vessel area (area within external elastic lamina), neointimal, medial, total wall areas (intima+media) and luminal area were calculated using image-analysis software (Image-Pro Plus version 4, Media Cybernetic, L.P.) as described previously (see Angelini G D, Lloyd C, Bush R, Johnson J, Newby A C. An external, oversized, porous polyester stent reduces vein graft neointima formation, cholesterol concentration, and vascular cell adhesion molecule 1 expression in cholesterol-fed pigs. J Thorac Cardiovasc Surg. 2002; 124:950-956; George S J, Izzat M B, Gadsdon P, Johnson J L, Yim A P, Wan S, Newby A C, Angelini G D, Jeremy J Y. Macro-porosity is necessary for the reduction of neointimal and medial thickening by external stenting of porcine saphenous vein bypass grafts. Atherosclerosis. 2001; 155:329-6; and Rajathurai T et al., supra).

Evaluation of neoangiogenesis within grafts was achieved using an ICC stain for biotinylated dolichos biflorus agglutinin (DBA) lectin (Vector Laboratories, Peterborough, U.K.) a component of the vascular endothelial glycocalyx as previously described (Rajathurai T et al., supra). Neoangiogenesis within the graft wall was determined by calculating the mean number of DBA lectin stained microvessels as counted in 4 fields at ×10 magnifications in 4 sections per graft. Four sections per graft were assessed. The number of vessles was expressed per mm². Inflammatory cell infiltration was determined by ICC for MAC 387 (Dako Laboratories, High Wycombe, Bucks, UK) with staining and quantification as per the DBA lectin protocol. Picrosirius red staining and quantitation of collagen density was performed as described previously.

### Power Calculations and Statistical Analysis

Neointimal area at 4 weeks was the primary endpoint. From previous work the inventors have found that that 6 pig vein-grafts in each group are required to demonstrate a biologically significant 50% reduction in neointimal area with power of 0.05 and an a value of 0.9. For the purposes of the present experiment to take into account unforeseen deaths in this recovery model the inventors used 12 animals; n=8 grafts per group. Categorical data was compared using the Chi-squared test where expected values were less than five. Continuous data was compared using ANOVA with unpaired Student's t-tests adjusted for multiple comparisons (Bonferroni) for intergroup comparisons. Effect sizes are expressed throughout as the percentage difference as well as the mean difference (95% confidence intervals). Values were considered significant if p was less than 0.05.

### Results

### Adverse Events and Toxicity

The initial experiments evaluated inventive microcapsules (CellBeads®) at a dose of 500,000 cells per graft or an estimated 20,000 cells.cm⁻² of adventitia. Grafts receiving non-stem cell containing alginate only beads or no treatment served as controls. There was no difference between baseline weights and there was no difference in the rate of weight gain between Cellbead® only and other groups. Three animals died of graft rupture during the study as follows; Cellbead® only (n=1), Control bead only (n=1) and contralateral CellBeads® and alginate only beads in one animal suspected of malignant hyperpyrexia (n=1). Graft rupture typically occurred at 5-7 days post grafting. At post-mortem, oedema and haemorrhage meant that it was difficult to determine with certainty the site of rupture in all cases. In two cases the rupture site was identified as being immediately adjacent to the venous side of the vascular anastomoses in CellBead® (n=1) and alginate only (n=1) treated grafts. One graft from eight (12.5%) in the Cellbead® group was occluded at harvest compared to 7 out of 8 (87.5%) in the alginate only group and none in the no treatment group (p<0.0001). On this basis therefore inventive microcapsules (CellBeads®) were compared to the no treatment controls for histomorphometric outcome measures.

### Vein Graft Morphology

In 4 week vein grafts, inventive microcapsules (CellBeads®) significantly decreased neointimal area by 57% (mean difference 3.54 mm², 95% CI 1.14-5.94, p=0.008) and total wall area by 21% (mean difference 2.71 mm², 95% CI 0.40-5.00, p=0.025) relative to untreated controls (FIGS. 2A, B). There was no difference in medial area, luminal area or total vessel area between groups.

### Angiogenesis, Inflammation, Thrombosis

There was a 68% increase in vein graft adventitial neoangiogenesis in the Cellbead® treated group (inventive microcapsules (CellBeads®)) compared to untreated grafts (mean difference 18.0 vessels/mm² 95% CI 5.49 to 30.53, p=0.004). There was also a 61% in the vessel count in CellBeads® treated grafts relative to alginate only (mean difference 17.1 vessels/mm² 95% CI 4.53 to 29.57, p=0.006). There was no difference in vein graft medial collagen density as determined by picrosirius red staining between the groups. Macrophages were detected in the walls of alginate only and Cellbead® treated vein grafts, but not in untreated grafts.

### Discussion

### Main Findings

1. In the pig model, peri-adventitial application of inventive microcapsules (CellBeads®) at a dose of 20000 cells.cm⁻² inhibited vein graft neointima formation compared to untreated controls.
2. Inhibition of neointima formation was associated with an increase in neoadventitial angiogenesis.
3. Graft fibrosis and inflammation were not significantly altered by treatment with inventive microcapsules (CellBeads) or alginate treatment.

### Study Strengths and Limitations

This is the first experiment in the prior art that has demonstrated that neointima formation in porcine vein grafts may be inhibited by the periadventitial application of pro-angiogenic immortalised human stem cells at the time of graft implantation. This represents a novel therapeutic strategy for the prevention of vascular diseases in general and in particular of vein graft diseases. It also represents a novel therapeutic role for human stem cells. Combined with demonstrable efficacy in the porcine model, where grafts have comparable diameter and wall thickness, the porcine grafts are exposed to similar haemodynamic stresses as human vein grafts, and develop neointimal thickening over a comparable time frame, 3-6 months, there is significant translational potential for this technique (Angelini G D, Bryan A J, Williams H M J, Soyombo A A, Williams A, Tovey J, Newby A C. Timecourse of medial and intimal thickening in pig arteriovenous bypass grafts: relationship to endothelial injury and cholesterol accumulation. J Thorac Cardiovasc Surg. 1992; 103:1093-1103; Higuchi Y, Hirayama A, Shimizu M, Sakakibara T, Kodama K. Postoperative changes in angiographically normal saphenous vein coronary bypass grafts using intravascular ultrasound. Heart Vessels. 2002; 17:57-60; Murphy G J, Angelini G D. Insights into the pathogenesis of vein graft disease: lessons from intravascular ultrasound. Cardiovascular Ultrasound 2004; 2:8).

### Summary

This is the first experiment in the prior art to investigate the effects of local peri-adventitial application of immortalised human stem cells that release pro-angiogenic peptides over a sustained period, on vein graft disease, wherein the results demonstrate significant efficacy. As a conclusion, induction of accelerated neoangiogenesis by periadventitial human stem cells inhibits vein graft wall thickening in porcine saphenous vein to carotid artery interposition grafts after 4 weeks and provides a reasonable basis for the application in vein greft diseases and in the prophylaxis, treatment, and amelioration of vascular diseases in general.

### Example 13

### Treatment in a Peripheral Vascular Disease Model

The objective of this study is to evaluate the recovery of mice receiving vehicle vs CellBeads secreting GLP-1 and VEGF vs CellBeads containing MSC secreting VEGF without GLP-1 in a murine hind limb ischemia model. Mice will be monitored for 21 days and then sacrificed for immunohistochemical characterization of neovascularization. Recovery will be assessed by Doppler (to measure limb perfusion) and histology (neovascularisation). The total number of animals for this study is 40 BALB/c mice.

### Example 14

### Perivascular Application of Micro-CellBeads after Peripheral Limb Ischemia

The objective of this study is to evaluate wether the perivascular application of Micro-CellBeads is feasible in a mice model of peripheral limb ischemia and whether perivascular applied Micro-CellBeads can induce an angiogenic response.

### Methods

### Animals

Male CD1 mice aged between 8 and 10 weeks were used for this study. All procedures had local ethical approval, were performed under UK government licence (Animals (Scientific Procedures) Act 1986), and conform to the Guide for the Care and Use of Laboratory Animals published by the US National Institutes of Health (NIH Publication No. 85-23, revised 1996). In two groups of animals peripheral limb ischemia was induced. The first group of these animals was treated with Micro-CellBeads. The second group was treated with vehicle only as a control. A third group of animals without induced peripheral limb ischemia (sham) was used as control as well. Each group consisted of 5 animals. One animal of each group was sacrificed one day after surgery and application of Micro-CellBeads. The other 4 animals were sacrificed one week after surgery.

### Preparation of Micro-CellBeads

Micro-CellBeads having an outer diameter of about 200 μm were used. About 40 cells were encapsulated in one Micro-CellBead and about 20,000 sterile and endotoxine free Micro-CellBeads were contained in a volume of 100 Micro-CellBeads were stored in the vapour phase of liquid nitrogen or at −80° C.

Directly before use, the beads were thawed and washed once with an appropriate medium (wash-solution) such as Ringer-solution or phosphate buffered saline (PBS) to get rid of the cryoprotective agent DMSO. The wash-solution contained about 2 mM calcium. The cryopreserverd vial containing Micro-CellBeads was thawed by incubation in a 37° C. waterbath or directly in the warm hand under visual inspection. The vial was inverted every 10 seconds to ensure the medium is liquid. As soon as the last ice was thawed the content of the vial was transferred into a sieving net, which was placed into a petri dish of 10 cm diameter containing 25 ml wash solution. For washing the Micro-CellBeads the petri dish was rotated for 1 min. Thereby the cryoprotectant was washed out. Thereafter, the sieving net was transferred into a new petri dish of 10 cm diameter containing 25 ml wash-solution. Again, the petri dish with the sieving net was rotated for about 1 min.

After washing the Micro-CellBeads the content of the sieving net was aspirated into a syringe intended to be used for application. Within the example the syringe was a 1 ml syringe. To pic up all of the material, the beads have to be concentrated into one section by carefully lifting the sieving net. For getting rid of surplus washing solution within the aspirated Micro-CellBeads the syringe was stored on the plunger for 5 minutes. This allows sedimentation of the Micro-CellBeads on the stopper of the plunger while surplus wash-solution forms the supernatant. Consequently, surplus wash-solution can easily be ejected before injection of the Micro-CellBeads.

The Micro-CellBeads were then ready for injection. They could be stored in the syringe for up to 3 hours. To eject the full Micro-CellBead volume of the syringe it is advantageous to have an air bubble on the stopper.

Peripheral Limb Ischemia and Treatment with Micro-CellBeads, Vehicle-Control

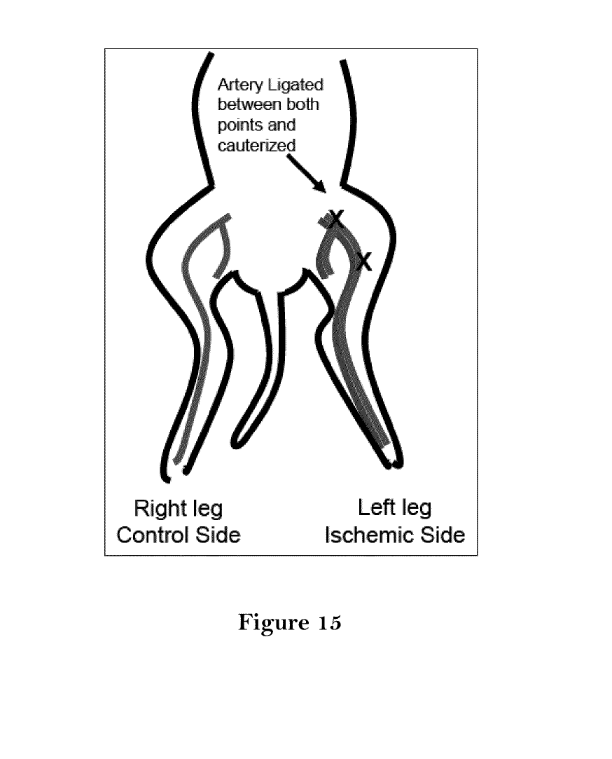

Peripheral Limb Ischemia was induced in the mice by femoral artery ligation. Therefore, male CD1-mice underwent operative ligation and electrocoagulation of the left common femoral artery proximal to the bifurcation of the superficial and deep artery, as is shown schematically by FIG. 15. Surgery was performed under tribomethanol anesthesia (880 mml/kg i.p., Sigma-Aldrich).

Immediately after the occlusion, 40 μl of Micro-CellBeads were administered to the first group of animals. Administration occurred into the perivascular space around the femoral artery and vein. To the second group of animals 40 μl of vehicle were administered into the perivascular space around the femoral artery and vein.

The limbs of terminally anesthetized mice (as described above: 5 mice per group, first mous was anestetized one day after surgergy the other ones seven days after surgery) were perfusion-fixed and the adductor muscles were harvested and paraffin embedded as described below to perform histological analyses of capillary and arteriolar densities.

### Sample Collection and Histology

Mice were anesthetized (Tribromoethanol), the abdominal cavity was opened and the aorta was cannulated in the direction of the limbs with a PE-50-catheter connected to a perfusion apparatus. The vasculature of adductor muscles was perfused with a heparinized PBS-solution at a pressure similar to the mean arterial pressure, followed by 10 min perfusion with 10% formalin. Ischemic and contralateral muscles were then removed, kept in 4% buffered formalin for 24 h and processed for paraffin embedding. Histological analysis was performed in adductor transverse sections (5 m in thickness) in a blinded fashion.

Hematoxyloin and Eosin staining was performerd for determining the persistence of Micro-CellBeads according to standard protocols.

Fluorescent immune-histochemical detection of Isolectin (by use of an antibody recognizing isolectin B4; Sigma) and α-smooth muscle actin (by use of an antibody recognizing α-SMA, Sigma) was performed to identify the number and density of arterioles and capillaries within the vicinity of the femoral artery. Immunhistological detection of Isolectin shows endothelial cells and thus allows to identify capillaries within histological sections of the collected samples while α-smooth muscle actin is a marker for smooth muscle cells and can be used to identify arterioles. DAPI-staining was performed as a control to show the nuclei of the cells within the histological sections. Slides were observed under a fluorescence microscope. Arterioles were recognized as vessels with one or more continuous layer of α-SMA-positive vascular smooth muscle cells and isolectin B4 positive lumen. The number of arterioles per mm² was counted. The number of capillaries per mm² was evaluated by counting the number of isolectin B4-positive and α-SMA-negative microvessels.

### Results

### Persistence of the Micro-CellBeads

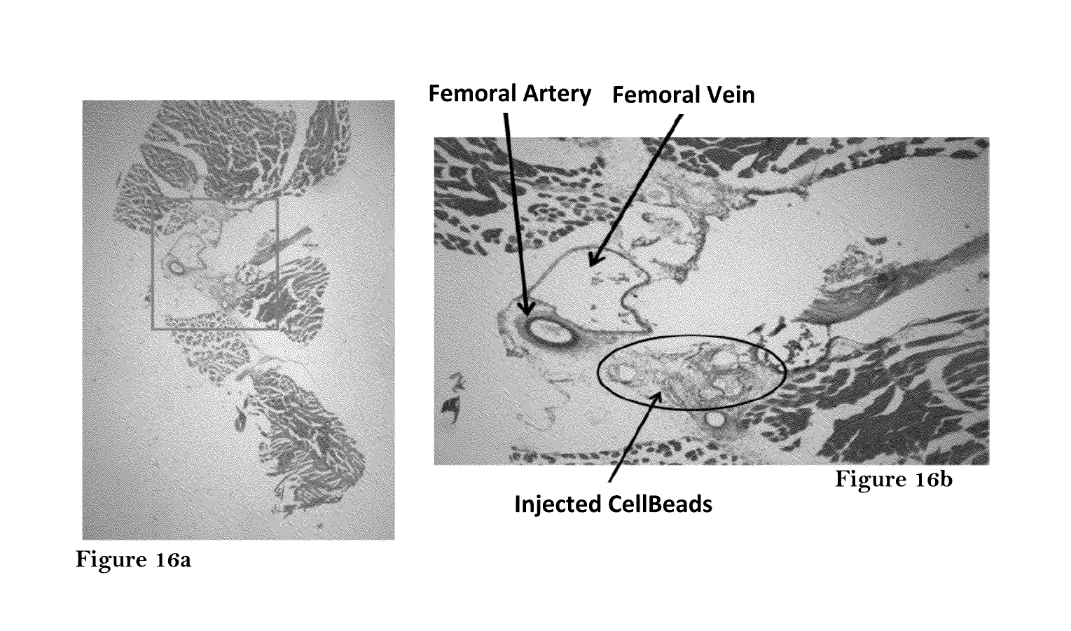

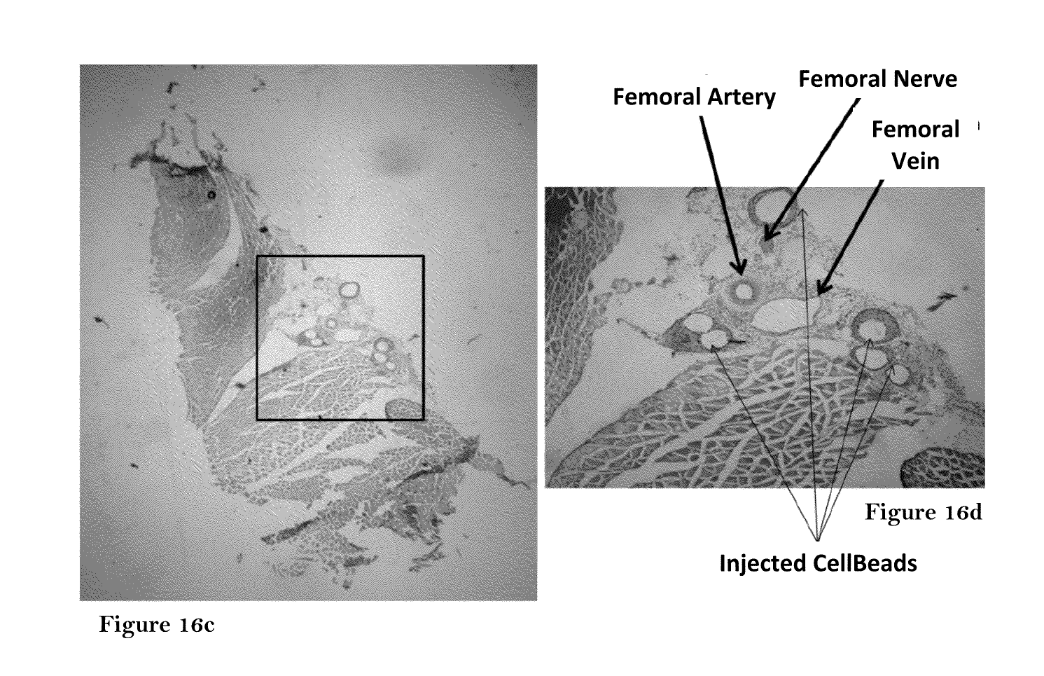

For the samples collected one day after surgery the presence of the Micro-CellBeads was observable by Hematoxylin and Eosin staining. The Micro-CellBeads were of irregular shape and not yet stabilized within the tissue, as can be seen in FIG. 16 A and FIG. 16 B. This is mainly due to the fact that they had not been in place for a sufficient period of time.

Hematoxylin and eosin staining of the samples collected after one week of surgery and Micro-CellBead administration showed the persistence of Micro-CellBeads surrounding the femoral artery and vein, as can be seen in FIG. 16 C and FIG. 16 D. The Micro-CellBeads still contained the MSCs.

### Angiogenic Response Induced by Micro-CellBeads

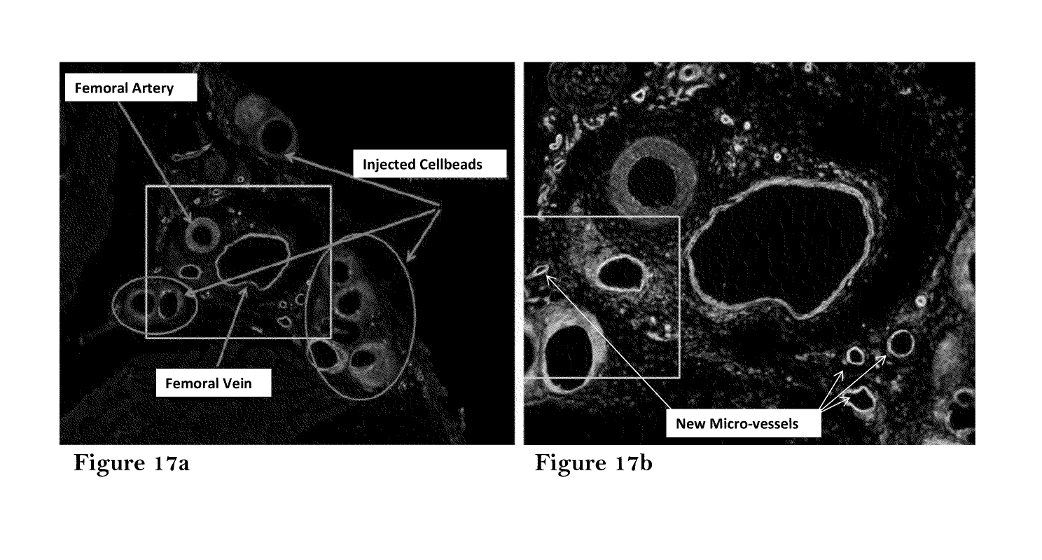

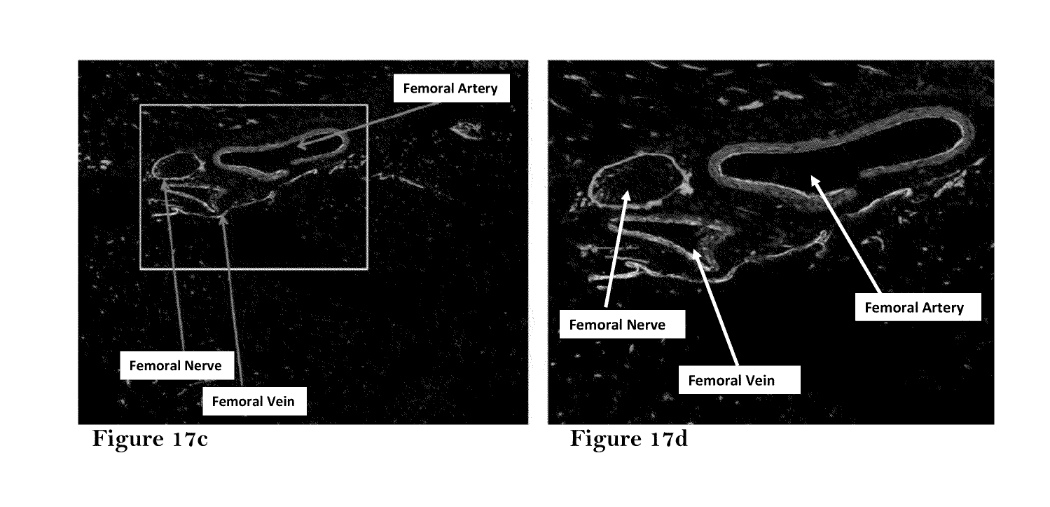

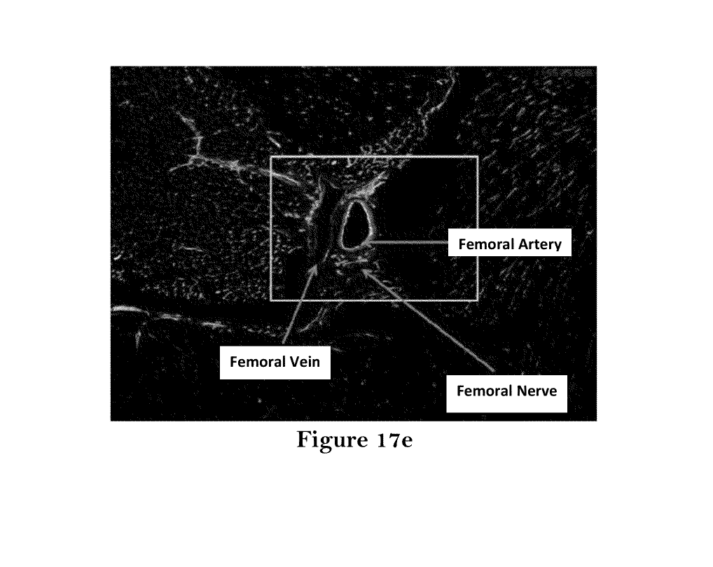

For the group of Micro-CellBead treated animals, immunhistochemical detection of Isolectin and a-smooth muscle actin showed a numer of small new micro-vessels between the femoral artery and the Micro-CellBeads, as can bee seen in FIG. 17 A and FIG. 17 B. This was neither seen in the sham-group nor in the animal-group treated with vehicle only, as can be seen in FIG. 17 C or FIG. 17 D (vehicle treatment) resp. FIG. 17 E (sham).

These results confirm the angiogenic potential of Micro-CellBeads after perivascular application. Further, feasibility of the technique of perivascular application of inventive Micro-CellBeads is confirmed.

1. A method for preparing a medicament for a treatment of vascular diseases using cells encoding and secreting at least GLP-1, a fragment or variant thereof, and additionally secreting VEGF, wherein the cells are encapsulated in a spherical microcapsule to prevent a response of an immune system of a patient to be treated.

2. The method of claim 1, wherein the spherical microcapsule comprises a spherical core and at least one surface coating layer, wherein the spherical core comprises a mixture of cross-linked polymers and cells, encoding and secreting at least GLP-1, a fragment or variant thereof, and additionally secreting VEGF; and wherein the at least one surface coating layer comprises cross-linked polymers.

3. The method of claim 1, wherein the spherical microcapsule has a total diameter of about 100 μm to about 800 μm.

4. The method of claim 1, wherein the cells are mesenchymal stem cells, mesenchymal stromal cells, human mesenchymal stem cells, differentiated cells derived from human mesenchymal stem cells, allogenic cells or autologous cells encoding and secreting at least GLP-1, a fragment or variant thereof, and additionally secreting VEGF.

5. The method of claim 2, wherein the cross-linked polymers comprise biopolymers and alginates.

6. The method of claim 2, wherein the cross-linked polymers of the core and/or the at least one surface coating layer comprise a chemically identical polymer in identical or differing concentrations, wherein the polymers further may have different molecular weights and/or may be cross-linked differently.

7. The method of claim 2, wherein the spherical microcapsule comprises 1, 2, 3, 4, or 5 or more surface coating layers.

8. The method of claim 1, wherein the spherical microcapsule further comprises an additional external surface coating layer consisting of polycations.

9. The method of claim 1, wherein GLP-1 is a peptide selected from the group consisting of:
(a) a peptide comprising aa 7-35 of GLP-1; or
(b) a peptide comprising aa 7-36 of GLP-1 or GLP-1(7-36)amide; or
(c) a peptide comprising aa 7-37 of GLP-1 or
(d) a peptide comprising the sequence according to formula II (SEQ ID NO: 51, SEQ ID NO: 52 or SEQ ID NO: 53); or
(e) a peptide comprising the sequence according to formula III (SEQ ID NO: 54, SEQ ID NO: 55 or SEQ ID NO: 56), or
(f) a peptide showing an identity of at least 80% with any of the herein peptides according to a) to e).

10. The method of claim 1, wherein GLP-1 is a GLP-1 fusion peptide or a fragment or variant thereof and comprises components (I) and (II), wherein an N-terminal of the component (I) comprises:
(a) a GLP-1(7-35, 7-36 or 7-37) sequence, or
(b) a sequence according to SEQ ID NO: 1; or
(c) a peptide comprising or consisting of the sequence according to formula II (SEQ ID NO: 51, SEQ ID NO: 52 or SEQ ID NO: 53); or
(d) a peptide comprising or consisting of the sequence according to formula III (SEQ ID NO: 54, SEQ ID NO: 55 or SEQ ID NO: 56); or
(e) or a sequence having at least 80% sequence identity with a sequence of any of sequence according to a) to d); and
a C-terminal of the component (II) is selected from a peptide sequence of at least 9 amino acids or a functional fragment or variant thereof.

11. The method of claim 10, wherein the component (II) of the GLP-1 fusion peptide is selected from:
(a) a peptide sequence containing a sequence according to SEQ ID NO: 22 (RRDFPEEVAI), SEQ ID NO: 27 (DFPEEVAI), SEQ ID NO: 28 (RDFPEEVA), or SEQ ID NO: 29 (RRDFPEEV), SEQ ID NO: 30 (AADFPEEVAI), SEQ ID NO: 31 (ADFPEEVA), or SEQ ID NO: 32 (AADFPEEV), or a sequence having at least 80% sequence identity with SEQ ID NOs: 22, 27, 28, 29, 30, 31 or 32; or
(b) a peptide sequence containing a sequence according to SEQ ID NO: 23 (RRDFPEEVAIVEEL) or SEQ ID NO: 24 (RRDFPEEVAIAEEL), or SEQ ID NO: 33 (AADFPEEVAIVEEL) or SEQ ID NO: 34 (AADFPEEVAIAEEL), or a sequence having at least 80% sequence identity with SEQ ID NOs: 23, 24, 33 or 34; or
(c) a peptide sequence containing a sequence according to SEQ ID NO: 2 (RRDFPEEVAIVEELG), SEQ ID NO: 3 (RRDFPEEVAIAEELG), SEQ ID NO: 35 (AADFPEEVAIVEELG), or SEQ ID NO: 36 (AADFPEEVAIAEELG), or a sequence having at least 80% sequence identity with SEQ ID NOs: 2, 3, 35 or 36.

12. The method of claim 10, wherein the component (I) and the component (II) of the GLP-1 fusion peptide are directly linked or linked via a linker sequence.

13. The method of claim 10, wherein the GLP-1 fusion peptide contains alternatively or additionally to the components (I) and (II) a component (III), wherein the component (III) may be linked to the C-terminus of component (I) and/or to the N-terminus of component (I), if components (I) and (III) are present in the fusion protein, or wherein the component (III) may be linked to the C-terminus of component (II) and/or to the N-terminus of component (I), if the components (I), (II) and (III) are present in the fusion protein.

14. The method of claim 13, wherein the component (III) comprises:
(a) the N-terminal sequence of GLP-2 as in proglucagon,
(b) a GLP-1(5-37, 6-37, or 7-37) sequence,
(c) a peptide comprising the sequence according to formula II (SEQ ID NO: 51, SEQ ID NO: 52 or SEQ ID NO: 53); or
(d) a peptide comprising a sequence according to formula III (SEQ ID NO: 54, SEQ ID NO: 55 or SEQ ID NO: 56); or
(e) or a sequence having at least 80% sequence identity with a sequence of any of sequence according to a) to d); or
(f) wherein component (III) contains the sequence of SEQ ID NOs: 4 or 5 or a sequence having at least 80% sequence identity with SEQ ID NOs: 4 or 5.

15. The method of claim 10, wherein the GLP-1 fusion peptide further comprises a carrier protein as component (IV), wherein the component IV is transferrin or albumin.

16. The method of claim 10, wherein the GLP-1 fusion peptide further comprises a peptide of SEQ ID NO: 6, SEQ ID NO: 7, SEQ ID NO: 8, SEQ ID NO: 10, SEQ ID NO: 11, SEQ ID NO: 12, SEQ ID NO: 26, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, or SEQ ID NO: 48, or a sequence having at least 80% sequence identity with SEQ ID NOs: 6, 7, 8, 10, 11, 12, 26, or 37 to 48.

17. The method of claim 1, wherein the cells in the core of the spherical microcapsules are engineered to additionally secrete a factor selected from the group consisting of anti-apoptotic factors, growth factors, erythropoietin (EPO), anti-platelet drugs, anti-coagulant drugs, and anti-thrombotic drugs, and/or secrete endogenous proteins or peptides as paracrine factors that are released through the capsule in therapeutic levels selected from IL6, IL8, GDNF, NT3, and MCP1.

18. The method of claim 1, wherein the vascular diseases, preferably peripheral vascular diseases (PVD), do not include cardiovascular diseases or diseases caused by stroke, (acute) myocardial infarct, heart failure, cardiomyopathy and/or coronary diseases.

19. The method of claim 1, wherein the vascular diseases are peripheral vascular disease, aneurysm, renal artery disease, Raynaud's phenomenon, Buerger's disease, peripheral venous disease, varicose veins, venous blood clots, deep vein thrombosis, pulmonary embolism, chronic venous insufficiency, vein graft disease or lymphedema, preferably peripheral vascular disease or vein graft disease.

20. The method of claim 3, wherein the spherical microcapsule has a total diameter of about 180 μm to about 800 μm.


SEQUENCE LISTING (56 sequences)

SEQ ID NO: 1 - protein, 31 aa, Artificial
  Description of sequence synthetic peptide corresponding to GLP-1(7-37)
HAEGTFTSDVSSYLEGQAAKEFIAWLVKGRG

SEQ ID NO: 2 - protein, 15 aa, Artificial
  Description of sequence full-length IP-2 sequence having all 15 amino acids of the naturally occurring IP-2 sequence, human.
RRDFPEEVAIVEELG

SEQ ID NO: 3 - protein, 15 aa, Artificial
  Description of sequence full-length IP-2 sequence having all 15 amino acids of the naturally occurring IP-2 sequence, murine.
RRDFPEEVAIAEELG

SEQ ID NO: 4 - protein, 35 aa, Artificial
  Description of sequence murine isoform of GLP-2
HADGSFSDEMNTILDNLAARDFINWLIQTKITDRK

SEQ ID NO: 5 - protein, 35 aa, Artificial
  Description of sequence human isoform of GLP-2
HADGSFSDEMSTILDNLATRDFINWLIQTKITDKK

SEQ ID NO: 6 - protein, 79 aa, Artificial
  Description of sequence SEQ ID No 6 (ID6syn, CM1) corresponds to GLP-1(7-37)-IP2-RR-GLP1(7-37), 79 aa, 8.7 kD
HAEGTFTSDVSSYLEGQAAKEFIAWLVKGRGRRDFPEEVAIAEELGRRHAEGTFTSDVSSYLEGQAAKEFIAWLVKGRG

SEQ ID NO: 7 - protein, 83 aa, Artificial
  Description of sequence SEQ ID No 7 (ID7rec, CM2) corresponds to GLP-1(7-37)-IP2-RR-GLP2, 83 aa, 9.4 kD
HAEGTFTSDVSSYLEGQAAKEFIAWLVKGRGRRDFPEEVAIAEELGRRHADGSFSDEMSTILDNLATRDFINWLIQTKITDKK

SEQ ID NO: 8 - protein, 46 aa, Artificial
  Description of sequence SEQ ID No8 (ID8 syn, CM3) corresponds to GLP-1(7-37)-IP2, 46 aa, 5.1 kD
HAEGTFTSDVSSYLEGQAAKEFIAWLVKGRGRRDFPEEVAIAEELG

SEQ ID NO: 9 - protein, 97 aa, Artificial
  Synthetic peptide, partial Homo sapiens matrix metallopeptidase 11 (stromelysin 3)
MAPAAWLRSAAARALLPPMLLLLLQPPPLLARALPPDVHHLHAERRGPQPWHAALPSSPAPAPATQEAPRPASSLRPPRCGVPDPSDGLSARNRQKR

SEQ ID NO: 10 - protein, 79 aa, Artificial
  Description of sequence SEQ ID No 10 (N-GLP-1(7-37)-IP2(human)-RR-GLP-1(7-37)-C, also designated human CM1)
HAEGTFTSDVSSYLEGQAAKEFIAWLVKGRGRRDFPEEVAIVEELGRRHAEGTFTSDVSSYLEGQAAKEFIAWLVKGRG

SEQ ID NO: 11 - protein, 83 aa, Artificial
  Description of sequence SEQ ID No 11 N-GLP-1(7-37)-IP2(human)-RR-GLP-2-C, also designated human CM2
HAEGTFTSDVSSYLEGQAAKEFIAWLVKGRGRRDFPEEVAIVEELGRRHADGSFSDEMNTILDNLAARDFINWLIQTKITDRK

SEQ ID NO: 12 - protein, 46 aa, Artificial
  Description of sequence SEQ ID No 12, GLP-1(7-37) linked without any linker sequence via its C-terminus to human IP2
HAEGTFTSDVSSYLEGQAAKEFIAWLVKGRGRRDFPEEVAIVEELG

SEQ ID NO: 13 - DNA, 815 bp, Artificial
  Description of sequence DNA sequence of the construct according to Fig. 1k
gatatccaccatggcccccgccgcctggctgaggagcgccgccgccagggccctgctgccacccatgctgctgctgctgctgcagcccccacctctgctggcccgggccctgcccccggtgagtgcccgccactcgccgtccgctcctcgctgagggggcgccgggcacgcgggctgggcccagcggcgtatccggacgccaagaaaccagagagccagccagatgccaaagggccctgccatgtgccggtgccctttccctctccatttgccctgccacacagtgggctggggttgcacgtgtgtttgctgacaggccacatctctaactgtgggccatgtggaccttaggcctgaccagaccctcatgtcttcctccttcccaggacgtgcaccacctgcacgccgagaggcgcggccctcagccctggcacgccgccctgccaagcagccctgcccctgccccagccacccaggaggcccccaggcctgccagcagcctgaggccacccaggtgcggcgtgcctgatccctccgatggcctgagcgctcggaatcggcagaagaggcacgccgagggcaccttcacctccgacgtgagcagctacctggagggccaggccgccaaggagttcatcgcctggctggtgaagggcaggggccgcagggacttccctgaggaggtggccatcgtggaggagctgggccggcgacacgccgagggcaccttcacctccgacgtgagcagctacctggagggccaggccgccaaggagttcatcgcctggctggtgaagggcaggggctgagcgcgc

SEQ ID NO: 14 - DNA, 815 bp, Artificial; identical to SEQ ID NO: 13
  Description of sequence DNA sequence of the construct according to Fig. 1k

SEQ ID NO: 15 - DNA, 834 bp, Artificial
  Description of sequence DNA sequence of the construct according to Fig. 1h
gatatccaccatggcccccgccgcctggctgaggagcgccgccgccagggccctgctgccacccatgctgctgctgctgctgcagcccccacctctgctggcccgggccctgcccccggtgagtgcccgccactcgccgtccgctcctcgctgagggggcgccgggcacgcgggctgggcccagcggcgtatccggacgccaagaaaccagagagccagccagatgccaaagggccctgccatgtgccggtgccctttccctctccatttgccctgccacacagtgggctggggttgcacgtgtgtttgctgacaggccacatctctaactgtgggccatgtggaccttaggcctgaccagaccctcatgtcttcctccttcccaggacgtgcaccacctgcacgccgagaggcgcggccctcagccctggcacgccgccctgccaagcagccctgcccctgccccagccacccaggaggcccccaggcctgccagcagcctgaggccacccaggtgcggcgtgcctgatccctccgatggcctgagcgctcggaatcggcagaagaggcacgccgagggcaccttcacctccgacgtgagcagctacctggagggccaggccgccaaggagttcatcgcctggctggtgaagggcaggggccgcagggacttccctgaggaggtggccatcgtggaggagctgggccggcgacacgccgacggcagcttcagcgacgagatgaacaccatcctggacaacctggccgcgcgcgacttcatcaactggctgatccagaccaagatcaccgatcggaagtgagcgcgctgatatc

SEQ ID NO: 16 - DNA, 834 bp, Artificial; identical to SEQ ID NO: 15
  Description of sequence DNA sequence of the construct according to Fig. 1h

SEQ ID NO: 17 - DNA, 780 bp, Artificial
  Description of sequence DNA sequence sequence and translated peptide sequence of the construct according to Fig. 1l
gatatccaccatggcccccgccgcctggctgaggagcgccgccgccagggccctgctgccacccatgctgctgctgctgctgcagcccccacctctgctggcccgggccctgcccccggtgagtgcccgccactcgccgtccgctcctcgctgagggggcgccgggcacgcgggctgggcccagcggcgtatccggacgccaagaaaccagagagccagccagatgccaaagggccctgccatgtgccggtgccctttccctctccatttgccctgccacacagtgggctggggttgcacgtgtgtttgctgacaggccacatctctaactgtgggccatgtggaccttaggcctgaccagaccctcatgtcttcctccttcccaggacgtgcaccacctgcacgccgagaggcgcggccctcagccctggcacgccgccctgccaagcagccctgcccctgccccagccacccaggaggcccccaggcctgccagcagcctgaggccacccaggtgcggcgtgcctgatccctccgatggcctgagcgctcggaatcggcagaagaggcacgccgagggcaccttcacctccgacgtgagcagctacctggagggccaggccgccaaggagttcatcgcctggctggtgaagggcaggggccgcagggacttccctgaggaggtggccatcgtggaggagctgggccggcgacacgccgacggcagcttcagcgacgagatgaacaccatcctggacaacctggccgcgcgctgatatc

SEQ ID NO: 18 - DNA, 720 bp, Artificial
  Description of sequence DNA sequence of the construct according to Fig. 1l
gatatccaccatggcccccgccgcctggctgaggagcgccgccgccagggccctgctgccacccatgctgctgctgctgctgcagcccccacctctgctggcccgggccctgcccccggtgagtgcccgccactcgccgtccgctcctcgctgagggggcgccgggcacgcgggctgggcccagcggcgtatccggacgccaagaaaccagagagccagccagatgccaaagggccctgccatgtgccggtgccctttccctctccatttgccctgccacacagtgggctggggttgcacgtgtgtttgctgacaggccacatctctaactgtgggccatgtggaccttaggcctgaccagaccctcatgtcttcctccttcccaggacgtgcaccacctgcacgccgagaggcgcggccctcagccctggcacgccgccctgccaagcagccctgcccctgccccagccacccaggaggcccccaggcctgccagcagcctgaggccacccaggtgcggcgtgcctgatccctccgatggcctgagcgctcggaatcggcagaagaggcacgccgagggcaccttcacctccgacgtgagcagctacctggagggccaggccgccaaggagttcatcgcctggctggtgaagggcagggacggcagcttcagcgacgagatgaacaccatcctggacaacctggccgcgcgctgatatc

SEQ ID NO: 19 - DNA, 716 bp, Artificial
  Description of sequence DNA sequence and sequence of the translated peptide according to Fig. 1m
gatatccaccatggcccccgccgcctggctgaggagcgccgccgccagggccctgctgccacccatgctgctgctgctgctgcagcccccacctctgctggcccgggccctgcccccggtgagtgcccgccactcgccgtccgctcctcgctgagggggcgccgggcacgcgggctgggcccagcggcgtatccggacgccaagaaaccagagagccagccagatgccaaagggccctgccatgtgccggtgccctttccctctccatttgccctgccacacagtgggctggggttgcacgtgtgtttgctgacaggccacatctctaactgtgggccatgtggaccttaggcctgaccagaccctcatgtcttcctccttcccaggacgtgcaccacctgcacgccgagaggcgcggccctcagccctggcacgccgccctgccaagcagccctgcccctgccccagccacccaggaggcccccaggcctgccagcagcctgaggccacccaggtgcggcgtgcctgatccctccgatggcctgagcgctcggaatcggcagaagaggcacgccgagggcaccttcacctccgacgtgagcagctacctggagggccaggccgccaaggagttcatcgcctggctggtgaagggcaggggccgcagggacttccctgaggaggtggccatcgtggaggagctgggctgagcgcgc

SEQ ID NO: 20 - DNA, 716 bp, Artificial; identical to SEQ ID NO: 19
  Description of sequence DNA sequence of the construct according to Fig. 1m

SEQ ID NO: 21 - protein, 31 aa, Artificial
  Synthetic glucagon like peptide
HGEGTFTSDVSSYLEGQAAKEFIAWLVKGRG

SEQ ID NO: 22 - protein, 10 aa, Artificial
  Description of sequence sequence contained in component (II) of a GLP-1 fusion peptide
RRDFPEEVAI

SEQ ID NO: 23 - protein, 14 aa, Artificial
  Description of sequence sequence contained in component (II) of a GLP-1 fusion peptide
RRDFPEEVAIVEEL

SEQ ID NO: 24 - protein, 14 aa, Artificial
  Description of sequence sequence contained in component (II) of a GLP-1 fusion peptide
RRDFPEEVAIAEEL

SEQ ID NO: 25 - protein, 30 aa, Artificial
  Sequence according to formula (I)
HAEGTFTSDVSSYLEGQAAKEFIAWLVKGR

SEQ ID NO: 26 - DNA, 584 bp, Artificial
  Description of sequence DNA sequence coding for GLP-1 fusion peptide of Fig 1e #217
aattcagataattcgatagccccgggcaccatggctcccgctgcatggctgagatctgcggccgcgcgcgccctcctgcccccgatgctgctgctgctgctccagccgccgccgctgctggcccgggctctgccgccggacgtccaccacctccatgccgagaggagggggccacagccctggcatgcagccctgcccagtagcccggcacctgcccctgccacgcaggaagccccccggcctgccagcagcctcaggcctccccgctgtggcgtgcccgacccatctgatgggctgagtgcccgcaaccgacagaagaggcatgccgaagggacctttaccagcgatgtgagctcttatctggaaggccaggctgccaaggagttcattgcttggctggtgaaaggccggggaaggcgggatttcccagaggaggtggccatcgtggaggagctgggccggcgacatgccgaagggacctttaccagcgatgtgagctcttatctggaaggccaggctgccaaggagttcattgcttggctggtgaaaggccggggatgaattgccaagggcgaattatcagg

SEQ ID NO: 27 - protein, 8 aa, Artificial Sequence
  Description of sequence sequence contained in component (II) of a GLP-1 fusion peptide
DFPEEVAI

SEQ ID NO: 28 - protein, 8 aa, Artificial Sequence
  Description of sequence sequence contained in component (II) of a GLP-1 fusion peptide
RDFPEEVA

SEQ ID NO: 29 - protein, 8 aa, Artificial Sequence
  Description of sequence sequence contained in component (II) of a GLP-1 fusion peptide
RRDFPEEV

SEQ ID NO: 30 - protein, 10 aa, Artificial Sequence
  Description of sequence sequence contained in component (II) of a GLP-1 fusion peptide
AADFPEEVAI

SEQ ID NO: 31 - protein, 8 aa, Artificial Sequence
  Description of sequence sequence contained in component (II) of a GLP-1 fusion peptide
ADFPEEVA

SEQ ID NO: 32 - protein, 8 aa, Artificial Sequence
  Description of sequence sequence contained in component (II) of a GLP-1 fusion peptide
AADFPEEV

SEQ ID NO: 33 - protein, 14 aa, Artificial Sequence
  Description of sequence sequence contained in component (II) of a GLP-1 fusion peptide
AADFPEEVAIVEEL

SEQ ID NO: 34 - protein, 14 aa, Artificial Sequence
  Description of sequence sequence contained in component (II) of a GLP-1 fusion peptide
AADFPEEVAIAEEL

SEQ ID NO: 35 - protein, 15 aa, Artificial Sequence
  Description of sequence sequence contained in component (II) of a GLP-1 fusion peptide
AADFPEEVAIVEELG

SEQ ID NO: 36 - protein, 15 aa, Artificial Sequence
  Description of sequence sequence contained in component (II) of a GLP-1 fusion peptide
AADFPEEVAIAEELG

SEQ ID NO: 37 - protein, 46 aa, Artificial Sequence
  Description of sequence sequence of a GLP-1 fusion peptide
HAEGTFTSDVSSYLEGQAAKEFIAWLVKGRGAADFPEEVAIAEELG

SEQ ID NO: 38 - protein, 46 aa, Artificial Sequence
  Description of sequence sequence of a GLP-1 fusion peptide
HAEGTFTSDVSSYLEGQAAKEFIAWLVKGRGAADFPEEVAIVEELG

SEQ ID NO: 39 - protein, 46 aa, Artificial Sequence
  Description of sequence sequence of a GLP-1 fusion peptide
HAEGTFTSDVSSYLEGQAAKEFIAWLVKGRGRRDFAEEVAIAEELG

SEQ ID NO: 40 - protein, 46 aa, Artificial Sequence
  Description of sequence sequence of a GLP-1 fusion peptide
HAEGTFTSDVSSYLEGQAAKEFIAWLVKGRGRRDAAAAVAIAEELG

SEQ ID NO: 41 - protein, 46 aa, Artificial Sequence
  Description of sequence sequence of a GLP-1 fusion peptide
HAEGTFTSDVSSYLEGQAAKEFIAWLVKGRGAADAAAAVAIAAALG

SEQ ID NO: 42 - protein, 36 aa, Artificial Sequence
  Description of sequence sequence of a GLP-1 fusion peptide
HAEGTFTSDVSSYLEGQAAKEFIAWLVKGRGRRDFP

SEQ ID NO: 43 - protein, 40 aa, Artificial Sequence
  Description of sequence sequence of a GLP-1 fusion peptide
HAEGTFTSDVSSYLEGQAAKEFIAWLVKGRGRRDFPEEVA

SEQ ID NO: 44 - protein, 51 aa, Artificial Sequence
  Description of sequence sequence of a GLP-1 fusion peptide
HAEGTFTSDVSSYLEGQAAKEFIAWLVKGRGRRDFPEEVAIAEELGRRHAC

SEQ ID NO: 45 - protein, 46 aa, Artificial Sequence
  Description of sequence sequence of a GLP-1 fusion peptide
HAEGTFTSDVSSYLEGQAAKEFIAWLVKGRGRRDFAEEVAIVEELG

SEQ ID NO: 46 - protein, 46 aa, Artificial Sequence
  Description of sequence sequence of a GLP-1 fusion peptide
HAEGTFTSDVSSYLEGQAAKEFIAWLVKGRGRRDAAAAVAIVEELG

SEQ ID NO: 47 - protein, 46 aa, Artificial Sequence
  Description of sequence sequence of a GLP-1 fusion peptide
HAEGTFTSDVSSYLEGQAAKEFIAWLVKGRGAADAAAAVAIVAALG

SEQ ID NO: 48 - protein, 51 aa, Artificial Sequence
  Description of sequence sequence of a GLP-1 fusion peptide
HAEGTFTSDVSSYLEGQAAKEFIAWLVKGRGRRDFPEEVAIVEELGRRHAC

SEQ ID NO: 49 - protein, 30 aa, Artificial; identical to SEQ ID NO: 25
  Sequence according to formula (I), GLP-1(7-36) amide

SEQ ID NO: 50 - protein, 31 aa, Artificial; identical to SEQ ID NO: 1
  Sequence according to formula (I), GLP(7-37) amide

SEQ ID NO: 51 - protein, 31 aa, Artificial Sequence
  Synthetic peptide according to formula (II)
HXEGTFTSDXSXXXEXXAXXXFIXWLXXGXX

SEQ ID NO: 52 - protein, 29 aa, Artificial Sequence
  Synthetic peptide according to formula (II)
HXEGTFTSDXSXXXEXXAXXXFIXWLXXG

SEQ ID NO: 53 - protein, 29 aa, Artificial Sequence
  Synthetic peptide according to formula (II)
HXEGTFTSDXSXXXEXXAXXXFIXWLXXG

SEQ ID NO: 54 - protein, 31 aa, Artificial Sequence
  Synthetic peptide according to formula (III)
HXEGTFTSDVSXYLEXXAAXEFIXWLVXGXX

SEQ ID NO: 55 - protein, 29 aa, Artificial Sequence
  Synthetic peptide according to formula (III)
HXEGTFTSDVSXYLEXXAAXEFIXWLVXG

SEQ ID NO: 56 - protein, 29 aa, Artificial Sequence
  Synthetic peptide according to formula (III)
HXEGTFTSDVSXYLEXXAAXEFIXWLVXG